SEQUENCE LISTING (56 sequences)

SEQ ID NO: 1 - DNA, 4627 bp, Homo sapiens
  source 1..4627: mol_type = genomic DNA
atatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacagtattctaaggacgccagaaagctgttgatcggcttcaagcaggggagggacacctaatttgcttttcttttttttttttttttttttttttttttttgagatggagttttgctcttgttgcccaggctggagtgcaatggtgcatcttggctcactgcaacctccgcctcccaggttcaagtgattctcctgcctcagcctcccgagtagctgagattacaggcacccgccaccatgcctggctaattttttgtatttttagtagagacagggtttcactatgttggccaggctggtctcgaactcctgacctcaggtgatccacccgcttcagcctcccaaagtgctgggattacaggcgtgagccaccacacccggcctgcttttcttaaagatcaatctgagtgctgtacggagagtgggttgtaagccaagagtagaagcagaaagggagcagttgcagcagagagatgatggaggcctgggcagggtggtggcagggaggtaaccaacaccattcaggtttcaaaggtagaaccatgcagggatgagaaagcaaagaggggatcaaggaaggcagctggattttggcctgagcagctgagtcaatgatagtgccgtttactaagaagaaaccaaggaaaaaatttggggtgcagggatcaaaactttttggaacatatgaaagtacgtgtttatactctttatggcccttgtcactatgtatgcctcgctgcctccattggactctagaatgaagccaggcaagagcagggtctatgtgtgatggcacatgtggccagggtcatgcaacatgtactttgtacaaacagtgtatattgagtaaatagaaatggtgtccaggagccgaggtatcggtcctgccagggccaggggctctccctagcaggtgctcatatgctgtaagttccctccagatctctccacaaggaggcatggaaaggctgtagttgttcacctgcccaagaactaggaggtctggggtgggagagtcagcctgctctggatgctgaaagaatgtctgtttttccttttagaaagttcctgtgatgtcaagctggtcgagaaaagctttgaaacaggtaagacaggggtctagcctgggtttgcacaggattgcggaagtgatgaacccgcaataaccctgcctggatgagggagtgggaagaaattagtagatgtgggaatgaatgatgaggaatggaaacagcggttcaagacctgcccagagctgggtggggtctctcctgaatccctctcaccatctctgactttccattctaagcactttgaggatgagtttctagcttcaatagaccaaggactctctcctaggcctctgtattcctttcaacagctccactgtcaagagagccagagagagcttctgggtggcccagctgtgaaatttctgagtcccttagggatagccctaaacgaaccagatcatcctgaggacagccaagaggttttgccttctttcaagacaagcaacagtactcacataggctgtgggcaatggtcctgtctctcaagaatcccctgccactcctcacacccaccctgggcccatattcatttccatttgagttgttcttattgagtcatccttcctgtggtagcggaactcactaaggggcccatctggacccgaggtattgtgatgataaattctgagcacctaccccatccccagaagggctcagaaataaaataagagccaagtctagtcggtgtttcctgtcttgaaacacaatactgttggccctggaagaatgcacagaatctgtttgtaaggggatatgcacagaagctgcaagggacaggaggtgcaggagctgcaggcctcccccacccagcctgctctgccttggggaaaaccgtgggtgtgtcctgcaggccatgcaggcctgggacatgcaagcccataaccgctgtggcctcttggttttacagatacgaacctaaactttcaaaacctgtcagtgattgggttccgaatcctcctcctgaaagtggccgggtttaatctgctcatgacgctgcggctgtggtccagctgaggtgaggggccttgaagctgggagtggggtttagggacgcgggtctctgggtgcatcctaagctctgagagcaaacctccctgcagggtcttgcttttaagtccaaagcctgagcccaccaaactctcctacttcttcctgttacaaattcctcttgtgcaataataatggcctgaaacgctgtaaaatatcctcatttcagccgcctcagttgcacttctcccctatgaggtaggaagaacagttgtttagaaacgaagaaactgaggccccacagctaatgagtggaggaagagagacacttgtgtacaccacatgccttgtgttgtacttctctcaccgtgtaacctcctcatgtcctctctccccagtacggctctcttagctcagtagaaagaagacattacactcatattacaccccaatcctggctagagtctccgcaccctcctcccccagggtccccagtcgtcttgctgacaactgcatcctgttccatcaccatcaaaaaaaaactccaggctgggtgcgggggctcacacctgtaatcccagcactttgggaggcagaggcaggaggagcacaggagctggagaccagcctgggcaacacagggagaccccgcctctacaaaaagtgaaaaaattaaccaggtgtggtgctgcacacctgtagtcccagctacttaagaggctgagatgggaggatcgcttgagccctggaatgttgaggctacaatgagctgtgattgcgtcactgcactccagcctggaagacaaagcaagatcctgtctcaaataataaaaaaaataagaactccagggtacatttgctcctagaactctaccacatagccccaaacagagccatcaccatcacatccctaacagtcctgggtcttcctcagtgtccagcctgacttctgttcttcctcattccagatctgcaagattgtaagacagcctgtgctccctcgctccttcctctgcattgcccctcttctccctctccaaacagagggaactctcctacccccaaggaggtgaaagctgctaccacctctgtgcccccccggcaatgccaccaactggatcctacccgaatttatgattaagattgctgaagagctgccaaacactgctgccaccccctctgttcccttattgctgcttgtcactgcctgacattcacggcagaggcaaggctgctgcagcctcccctggctgtgcacattccctcctgctccccagagactgcctccgccatcccacagatgatggatcttcagtgggttctcttgggctctaggtcctgcagaatgttgtgaggggtttatttttttttaatagtgttcataaagaaatacatagtattcttcttctcaagacgtggggggaaattatctcattatcgaggccctgctatgctgtgtatctgggcgtgttgtatgtcctgctgccgatgccttc

SEQ ID NO: 2 - DNA, 1448 bp, Homo sapiens
  source 1..1448: mol_type = genomic DNA
aggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcaggtgagtggggcctggggagatgcctggaggagattaggtgagaccagctaccagggaaaatggaaagatccaggtagcagacaagactagatccaaaaagaaaggaaccagcgcacaccatgaaggagaattgggcacctgtggttcattcttctcccagattctcagcccaacagagccaagcagctgggtcccctttctatgtggcctgtgtaactctcatctgggtggtgccccccatccccctcagtgctgccacatgccatggattgcaaggacaatgtggctgacatctgcatggcagaagaaaggaggtgctgggctgtcagaggaagctggtctgggcctgggagtctgtgccaactgcaaatctgactttacttttaattgcctatgaaaataaggtctctcatttattttcctctccctgctttctttcagactgtggctttacctcgggtaagtaagcccttccttttcctctccctctctcatggttcttgacctagaaccaaggcatgaagaactcacagacactggagggtggagggtgggagagaccagagctacctgtgcacaggtacccacctgtccttcctccgtgccaacagtgtcctaccagcaaggggtcctgtctgccaccatcctctatgagatcctgctagggaaggccaccctgtatgctgtgctggtcagcgcccttgtgttgatggccatggtaagcaggagggcaggatggggccagcaggctggaggtgacacactgacaccaagcacccagaagtatagagtccctgccaggattggagctgggcagtagggagggaagagatttcattcaggtgcctcagaagataacttgcacctctgtaggatcacagtggaagggtcatgctgggaaggagaagctggagtcaccagaaaacccaatggatgttgtgatgagccttactatttgtgtggtcaatgggccctactactttctctcaatcctcacaactcctggctcttaataacccccaaaactttctcttctgcaggtcaagagaaaggatttctga

SEQ ID NO: 3 - DNA, 1489 bp, Homo sapiens
  source 1..1489: mol_type = genomic DNA
aggacctgaaaaacgtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcaggtgagtggggcctggggagatgcctggaggagattaggtgagaccagctaccagggaaaatggaaagatccaggtagcggacaagactagatccagaagaaagccagagtggacaaggtgggatgatcaaggttcacagggtcagcaaagcacggtgtgcacttcccccaccaagaagcatagaggctgaatggagcacctcaagctcattcttccttcagatcctgacaccttagagctaagctttcaagtctccctgaggaccagccatacagctcagcatctgagtggtgtgcatcccattctcttctggggtcctggtttcctaagatcatagtgaccacttcgctggcactggagcagcatgagggagacagaaccagggctatcaaaggaggctgactttgtactatctgatatgcatgtgtttgtggcctgtgagtctgtgatgtaaggctcaatgtccttacaaagcagcattctctcatccatttttcttcccctgttttctttcagactgtggcttcacctccggtaagtgagtctctcctttttctctctatctttcgccgtctctgctctcgaaccagggcatggagaatccacggacacaggggcgtgagggaggccagagccacctgtgcacaggtgcctacatgctctgttcttgtcaacagagtcttaccagcaaggggtcctgtctgccaccatcctctatgagatcttgctagggaaggccaccttgtatgccgtgctggtcagtgccctcgtgctgatggccatggtaaggaggagggtgggatagggcagatgatgggggcaggggatggaacatcacacatgggcataaaggaatctcagagccagagcacagcctaatatatcctatcacctcaatgaaaccataatgaagccagactggggagaaaatgcagggaatatcacagaatgcatcatgggaggatggagacaaccagcgagccctactcaaattaggcctcagagcccgcctcccctgccctactcctgctgtgccatagcccctgaaaccctgaaaatgttctctcttccacaggtcaagagaaaggattccagaggctag

SEQ ID NO: 4 - protein, 140 aa, Homo sapiens
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 5 - protein, 176 aa, Homo sapiens
DLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 6 - protein, 178 aa, Homo sapiens
DLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 7 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 8 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 9 - protein, 41 aa, Homo sapiens
RSKRSRLLHSDYMNMTPRRPGPTRKHYQPYAPPRDFAAYRS

SEQ ID NO: 10 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 11 - protein, 42 aa, Homo sapiens
KRGRKKLLYIFKQPFMRPVQTTQEEDGCSCRFPEEEEGGCEL

SEQ ID NO: 12 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 13 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 14 - protein, 288 aa, Homo sapiens
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 15 - DNA, 2733 bp, Homo sapiens
  source 1..2733: mol_type = genomic DNA
ctcgcgaggccggctaggcccgaatgtcgttagccgtggggaaagatggcggaaaatttaaaaggctgcagcgtgtgttgcaagtcttcttggaatcagctgcaggacctgtgccgcctggccaagctctcctgccctgccctcggtatctctaagaggaacctctatgactttgaagtcgagtacctgtgcgattacaagaagatccgcgaacaggaatattacctggtgaaatggcgtggatatccagactcagagagcacctgggagccacggcagaatctcaagtgtgtgcgtatcctcaagcagttccacaaggacttagaaagggagctgctccggcggcaccaccggtcaaagaccccccggcacctggacccaagcttggccaactacctggtgcagaaggccaagcagaggcgggcgctccgtcgctgggagcaggagctcaatgccaagcgcagccatctgggacgcatcactgtagagaatgaggtggacctggacggccctccgcgggccttcgtgtacatcaatgagtaccgtgttggtgagggcatcaccctcaaccaggtggctgtgggctgcgagtgccaggactgtctgtgggcacccactggaggctgctgcccgggggcgtcactgcacaagtttgcctacaatgaccagggccaggtgcggcttcgagccgggctgcccatctacgagtgcaactcccgctgccgctgcggctatgactgcccaaatcgtgtggtacagaagggtatccgatatgacctctgcatcttccggacggatgatgggcgtggctggggcgtccgcaccctggagaagattcgcaagaacagcttcgtcatggagtacgtgggagagatcattacctcagaggaggcagagcggcggggccagatctacgaccgtcagggcgccacctacctctttgacctggactacgtggaggacgtgtacaccgtggatgccgcctactatggcaacatctcccactttgtcaaccacagttgtgaccccaacctgcaggtgtacaacgtcttcatagacaaccttgacgagcggctgccccgcatcgctttctttgccacaagaaccatccgggcaggcgaggagctcacctttgattacaacatgcaagtggaccccgtggacatggagagcacccgcatggactccaactttggcctggctgggctccctggctcccctaagaagcgggtccgtattgaatgcaagtgtgggactgagtcctgccgcaaatacctcttctagcccttagaagtctgaggccagactgactgagggggcctgaagctacatgcacctcccccactgctgccctcctgtcgagaatgactgccagggcctcgcctgcctccacctgcccccacctgctcctacctgctctacgttcagggctgtggccgtggtgaggaccgactccaggagtcccctttccctgtcccagccccatctgtgggttgcacttacaaacccccacccaccttcagaaatagtttttcaacatcaagactctctgtcgttgggattcatggcctattaaggaggtccaaggggtgagtcccaacccagccccagaatatatttgtttttgcacctgcttctgcctggagattgaggggtctgctgcaggcctcctccctgctgccccaaaggtatggggaagcaaccccagagcaggcagacatcagaggccagagtgcctagcccgacatgaagctggttccccaaccacagaaactttgtactagtgaaagaaaggggtccctggcctacgggctgaggctggtttctgctcgtgcttacagtgctgggtagtgttggccctaagagctgtagggtctcttcttcagggctgcatatctgagaagtggatgcccacatgccactggaagggaagtgggtgtccatgggccactgagcagtgagaggaaggcagtgcagagctggccagccctggaggtaggctgggaccaagctctgccttcacagtgcagtgaaggtacctagggctcttgggagctctgcggttgctaggggccctgacctggggtgtcatgaccgctgacaccactcagagctggaaccaagatctagatagtccgtagatagcacttaggacaagaatgtgcattgatggggtggtgatgaggtgccaggcactaggtagagcacctggtccacgtggattgtctcagggaagccttgaaaaccacggaggtggatgccaggaaagggcccatgtggcagaaggcaaagtacaggccaagaattgggggtgggggagatggcttccccactatgggatgacgaggcgagagggaagcccttgctgcctgccattcccagaccccagccctttgtgctcaccctggttccactggtctcaaaagtcacctgcctacaaatgtacaaaaggcgaaggttctgatggctgccttgctccttgctcccccaccccctgtgaggacttctctaggaagtccttcctgactacctgtgcccagagtgcccctacatgagactgtatgccctgctatcagatgccagatctatgtgtctgtctgtgtgtccatcccgccggccccccagactaacctccaggcatggactgaatctggttctcctcttgtacacccctcaaccctatgcagcctggagtgggcatcaataaaatgaactgtcgactgaaaaaaaaaa

SEQ ID NO: 16 - protein, 412 aa, Homo sapiens
MAENLKGCSVCCKSSWNQLQDLCRLAKLSCPALGISKRNLYDFEVEYLCDYKKIREQEYYLVKWRGYPDSESTWEPRQNLKCVRILKQFHKDLERELLRRHHRSKTPRHLDPSLANYLVQKAKQRRALRRWEQELNAKRSHLGRITVENEVDLDGPPRAFVYINEYRVGEGITLNQVAVGCECQDCLWAPTGGCCPGASLHKFAYNDQGQVRLRAGLPIYECNSRCRCGYDCPNRVVQKGIRYDLCIFRTDDGRGWGVRTLEKIRKNSFVMEYVGEIITSEEAERRGQIYDRQGATYLFDLDYVEDVYTVDAAYYGNISHFVNHSCDPNLQVYNVFIDNLDERLPRIAFFATRTIRAGEELTFDYNMQVDPVDMESTRMDSNFGLAGLPGSPKKRVRIECKCGTESCRKYLF

SEQ ID NO: 17 - protein, 90 aa, Homo sapiens
IQNPDPAVYQLRDSKSSDKSVCLFTDFDSQTNVSQSKDSDVYITDKTVLDMRSMDFKSNSAVAWSNKSDFACANAFNNSIIPEDTFFPSP

SEQ ID NO: 18 - protein, 42 aa, Homo sapiens
MPIWKFPDEEGACQPCPINCTHSCVDKDDKGCPAEQRASPLT

SEQ ID NO: 19 - protein, 15 aa, synthetic construct
GGGGSGGGGSGGGGS

SEQ ID NO: 20 - protein, 20 aa, Homo sapiens
MYRMQLLSCIALSLALVTNS

SEQ ID NO: 21 - protein, 20 aa, Mus musculus
MYSMQLASCVTLTLVLLVNS

SEQ ID NO: 22 - protein, 20 aa, Homo sapiens
METPAQLLFLLLLWLPDTTG

SEQ ID NO: 23 - protein, 20 aa, Mus musculus
METDTLLLWVLLLWVPGSTG

SEQ ID NO: 24 - protein, 21 aa, Homo sapiens
MALPVTALLLPLALLLHAARP

SEQ ID NO: 25 - protein, 18 aa, Homo sapiens
MALPVTALLLPLALLLHA

SEQ ID NO: 26 - protein, 16 aa, synthetic construct
MKWVTFISLLFSSAYS

SEQ ID NO: 27 - protein, 30 aa, synthetic construct
MDSKGSSQKGSRLLLLLVVSNLLLCQGVVS

SEQ ID NO: 28 - DNA, 519 bp, Mus musculus
  source 1..519: mol_type = genomic DNA
gaggacctgcggaatgtcacgcccccgaaagtgtccctgtttgaaccaagtaaagccgagattgcgaataagcagaaggctactctggtttgcttggcacgaggattttttcctgaccatgtggaactgagctggtgggttaatgggaaagaagttcattcaggcgtatgtactgatccacaggcctacaaggaatcaaattactcttactgtctctcttcccgattgcgcgtttctgctacattttggcacaatcctcgaaatcatttccggtgccaagttcaattccacggtttgtcagaggaagacaaatggcccgagggcagtcctaagccagtaacgcagaatatatcagctgaggcgtggggcagagctgactgtggtataacttcagcgagctatcagcaaggggttctgagtgctactatcctctacgagatcctgctagggaaggccaccctgtatgctgtgctggtctctactctcgtggttatggcaatggtgaaacggaaaaacagc

SEQ ID NO: 29 - protein, 173 aa, Mus musculus
EDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 30 - DNA, 408 bp, Mus musculus
  source 1..408: mol_type = genomic DNA
atccagaaccctgagcccgctgtgtaccaattgaaagacccacggtcacaggacagtaccttgtgcctcttcacggactttgactctcagatcaacgttcctaagacgatggaaagtggcacatttatcacggataagtgtgtcttggatatgaaagctatggatagcaagtctaatggtgctatcgcatggtcaaaccagacttccttcacctgccaggatatttttaaggaaaccaatgcaacttacccatctagcgatgtaccatgcgatgccaccctgacggagaaaagctttgaaacagatatgaacctaaactttcaaaacctgctggtgattgtactgcgaatcctcctcctgaaagtggccgggtttaatctgctcatgacgctgcggctgtggtccagc

SEQ ID NO: 31 - protein, 136 aa, Mus musculus
IQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 32 - DNA, 930 bp, synthetic construct
  source 1..930: mol_type = other DNA
atgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttaaacggggcagaaagaaactcctgtatatattcaaacaaccatttatgagaccagtacaaactactcaagaggaagatggctgtagctgccgatttccagaagaagaagaaggaggatgtgaactgtga

SEQ ID NO: 33 - protein, 309 aa, synthetic construct
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFKRGRKKLLYIFKQPFMRPVQTTQEEDGCSCRFPEEEEGGCEL

SEQ ID NO: 34 - DNA, 360 bp, synthetic construct
  source 1..360: mol_type = other DNA
gaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtc

SEQ ID NO: 35 - DNA, 318 bp, synthetic construct
  source 1..318: mol_type = other DNA
gacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgattcacaggcagtggatctgggacagatttcactctcaccatcactaacgtgcagtctaaagacttggcagactatttctgtcaacaatataacaggtatccgtacacgtccggaggggggaccaagctggagatc

SEQ ID NO: 36 - protein, 120 aa, synthetic construct
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTV

SEQ ID NO: 37 - protein, 106 aa, synthetic construct
DIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEI

SEQ ID NO: 38 - DNA, 354 bp, synthetic construct
  source 1..354: mol_type = other DNA
gaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtc

SEQ ID NO: 39 - DNA, 318 bp, synthetic construct
  source 1..318: mol_type = other DNA
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatc

SEQ ID NO: 40 - protein, 118 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTV

SEQ ID NO: 41 - protein, 106 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEI

SEQ ID NO: 42 - DNA, 339 bp, synthetic construct
  source 1..339: mol_type = other DNA
gaggtacagctacaacaatcaggacccgaattgaagaaaccgggtacatccgtgcgcatcagctgtaagacctccggttatacctttaccgaatataccatccattgggttaaacagagccacggcaaatcgctggaatggattggtaacatcaacccgaacaacggtggcaccacgtacaatcagaagttcgaggacaaagccaccctgacggttgacaagagctcttccactgcgtatatggaactgcgtagcctgaccagcgaggacagcgcggtgtactactgcgcggctggttggaattttgattactggggtcaaggtacgaccttgaccgtc

SEQ ID NO: 43 - DNA, 318 bp, synthetic construct
  source 1..318: mol_type = other DNA
gatattgttatgacccagagccacaaattcatgagcacctctgtgggtgatcgcgtgtccatcatctgcaaggcgagccaggatgttggtactgccgtcgactggtaccagcaaaaaccgggtcaatccccaaaattgctgatttactgggccagcacgcgtcataccggggtgccggatcgttttaccggttctggcagcggcactgacttcacgctgaccattaccaatgttcagtcggaggacctggcagactacttctgccaacaatataacagctatccgctgacctttggtgcaggtacgatgttagatctt

SEQ ID NO: 44 - protein, 113 aa, synthetic construct
EVQLQQSGPELKKPGTSVRISCKTSGYTFTEYTIHWVKQSHGKSLEWIGNINPNNGGTTYNQKFEDKATLTVDKSSSTAYMELRSLTSEDSAVYYCAAGWNFDYWGQGTTLTV

SEQ ID NO: 45 - protein, 106 aa, synthetic construct
DIVMTQSHKFMSTSVGDRVSIICKASQDVGTAVDWYQQKPGQSPKLLIYWASTRHTGVPDRFTGSGSGTDFTLTITNVQSEDLADYFCQQYNSYPLTFGAGTMLDL

SEQ ID NO: 46 - DNA, 420 bp, synthetic construct
  source 1..420: mol_type = other DNA
atccagaaccccgaccctgccgtctaccagctgagagattcaaaatcctccgacaaatccgtctgtctcttcaccgacttcgactcacagacaaatgtgtcccagtccaaggatagtgacgtgtacatcaccgacaaaactgtgctggacatgagatcaatggatttcaaatccaactccgccgtggcatggagtaacaaatccgatttcgcctgtgccaatgccttcaacaactccatcatccctgaggacaccttttttccatctcccgaatcatcttgtgacgtgaaactcgtcgagaaatcattcgaaaccgacaccaacctcaactttcagaatctgtccgtcatcggctttaggattctgctgctgaaagtggccggattcaatctgctcatgaccctgagactgtggtcatct

SEQ ID NO: 47 - DNA, 420 bp, synthetic construct
  source 1..420: mol_type = other DNA
attcagaatcctgatcctgccgtctaccagctgagagattcaaaatcctccgacaaatccgtctgtctcttcaccgacttcgactcacagacaaatgtgtcccagtccaaggatagtgacgtgtacatcaccgacaaaactgtgctggacatgagatcaatggatttcaaatccaactccgccgtggcatggagtaacaaatccgatttcgcctgtgccaatgccttcaacaactccatcatccctgaggacaccttttttccatctcccgaatcatcttgtgacgtgaaactcgtcgagaaatcattcgaaaccgacaccaacctcaactttcagaatctgtccgtcatcggctttaggattctgctgctgaaagtggccggattcaatctgctcatgaccctgagactgtggtcatct

SEQ ID NO: 48 - protein, 140 aa, synthetic construct; identical to SEQ ID NO: 4

SEQ ID NO: 49 - DNA, 540 bp, synthetic construct
  source 1..540: mol_type = other DNA
ctggaggatctgaaaaacgtgttccctcctgaagtggctgtctttgaaccatccgaggccgagatttcccatacccagaaagcaactctggtctgtctggccactggattctaccccgatcacgtggaactgtcttggtgggtgaacggcaaggaagtccattccggagtctctaccgaccctcagcccctcaaggagcagcctgctctcaacgattctcggtactgcctgtcatctcgactgagagtgtctgccaccttctggcagaaccctagaaaccactttcggtgtcaggtccagttttacggcctgagcgagaacgatgagtggacacaggatagagccaaacctgtgacacagattgtgagcgctgaggcttggggacgagccgattgtggcttcacatccgagtcttaccagcagggagtgctgtctgctacaatcctctacgaaattctcctggggaaggccaccctgtacgctgtcctcgtgtctgctctggtgctcatggctatggtcaaacgaaaggactctagaggc

SEQ ID NO: 50 - protein, 180 aa, synthetic construct
LEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 51 - DNA, 66 bp, synthetic construct
  source 1..66: mol_type = other DNA
ggctctggcgctaccaatttttccctcctcaaacaggctggagatgtcgaagagaaccccggacct

SEQ ID NO: 52 - DNA, 63 bp, synthetic construct
  source 1..63: mol_type = other DNA
atggctctcccagtgactgccctactgcttcccctagcgcttctcctgcatgcagccaggccg

SEQ ID NO: 53 - DNA, 801 bp, synthetic construct
  source 1..801: mol_type = other DNA
atgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgcttt

SEQ ID NO: 54 - protein, 267 aa, synthetic construct
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCF

SEQ ID NO: 55 - DNA, 925 bp, synthetic construct
  source 1..925: mol_type = other DNA
cgaggcagggctttggctactggagatcgtaggttcgaatcccgtctgggaagttcaacttgtgcacctgtaaaagaagctggcattattggcttgtactcaagggctggcacagagtgtgtcggggtgcggacgccccagccacgcccatcatccgtgcggaagatgcagaggtcatatcggatacccttctgtaccacacgatttgggcagtcatagccgcagcggcagcgggagttgcactcgtagatgggcagcccggctcgaagccgcacctggccctggtcattgtaggcaaacttgtgcagtgacgcccccgggcagcagcctccagtgggtgcccacagacagtcctggcactcgcagcccacagccacctggttgagggtgatgccctcaccaacacggtactcattgatgtacacgaaggcccgcggagggccgtccaggtccacctcattctctacagtgatgcgtcccagatggctgcgcttggcattgagctcctgctcccagcgacggagcgcccgcctctgcttggccttctgcaccaggtagttggccaagcttgggtccaggtgccggggggtctttgaccggtggtgccgccggagcagctccctttctaagtccttgtggaactgcttgaggatacgcacacacttgagattctgccgtggctcccaggtgctctctgagtctggatatccacgccatttcaccagggtcaaaggagaaaattccctttggaaacagatgtgggcagttggggacaagagggcaggacactaacttccttgtgacctgtcccctcccagagcatggtcaccccagactcacgcggatcttcttgtaatcgcacaggtactcgacttcaaagtcatagaggttcctcttagagataccgagggcagggcaggagagc

SEQ ID NO: 56 - RNA, 925 nt, synthetic construct
  source 1..925: mol_type = other RNA
cgaggcagggctttggctactggagatcgtaggttcgaatcccgtctgggaagttcaacttgtgcacctgtaaaagaagctggcattattggcttgtactcaagggctggcacagagtgtgtcggggtgcggacgccccagccacgcccatcatccgtgcggaagatgcagaggtcatatcggatacccttctgtaccacacgatttgggcagtcatagccgcagcggcagcgggagttgcactcgtagatgggcagcccggctcgaagccgcacctggccctggtcattgtaggcaaacttgtgcagtgacgcccccgggcagcagcctccagtgggtgcccacagacagtcctggcactcgcagcccacagccacctggttgagggtgatgccctcaccaacacggtactcattgatgtacacgaaggcccgcggagggccgtccaggtccacctcattctctacagtgatgcgtcccagatggctgcgcttggcattgagctcctgctcccagcgacggagcgcccgcctctgcttggccttctgcaccaggtagttggccaagcttgggtccaggtgccggggggtctttgaccggtggtgccgccggagcagctccctttctaagtccttgtggaactgcttgaggatacgcacacacttgagattctgccgtggctcccaggtgctctctgagtctggatatccacgccatttcaccagggtcaaaggagaaaattccctttggaaacagatgtgggcagttggggacaagagggcaggacactaacttccttgtgacctgtcccctcccagagcatggtcaccccagactcacgcggatcttcttgtaatcgcacaggtactcgacttcaaagtcatagaggttcctcttagagataccgagggcagggcaggagagc

(19) United States
(12) Patent Application Publication
(10) Pub. No.: US 2025/0213616 A1
(43) Pub. Date: Jul. 3, 2025

(54) COMPOSITIONS AND METHODS FOR TREATING A REFRACTORY OR RELAPSED CANCER OR A CHRONIC INFECTIOUS DISEASE

(71) Applicants: MNEMO THERAPEUTICS, Paris (FR); INSTITUT CURIE, Paris (FR); INSTITUT NATIONAL DE LA SANTE ET DE LA RECHERCHE MEDICALE, Paris (FR)

(72) Inventors: Michael SAITAKIS, Paris (FR); Sebastian AMIGORENA, Paris (FR); Jaime Rodrigo FUENTEALBA, Paris (FR); Sheila LOPEZ COBO, Paris (FR); Marion ALCANTARA, Paris (FR); Kyriaki TSALKITZI, Paris (FR); Ariane BIQUAND, Paris (FR); Armelle BOHINEUST, Paris (FR)

(21) Appl. No.: 18/867,177

(22) PCT Filed: May 22, 2023
(86) PCT No.: PCT/EP2023/063702

§ 371 Date: Nov. 19, 2024

(30) Foreign Application Priority Data
May 20, 2022 (EP) 22305760.5

(51) Int. Cl. A61K 35/17; A61K 40/11; A61K 40/15; A61K 40/31; A61K 40/32; A61K 40/42; A61K 45/06; A61P 35/00; C07K 14/705; C07K 14/725; C07K 14/735; C12N 9/22; C12N 15/11

(52) U.S. Cl. CPC A61K 35/17; A61K 40/11; A61K 40/15; A61K 40/31; A61K 40/32; A61K 40/4211; A61K 40/4212; A61K 45/06; A61P 35/00; C07K 14/7051; C07K 14/70517; C07K 14/70521; C07K 14/70532; C07K 14/70535; C07K 14/70575; C07K 14/70578; C07K 14/70589; C12N 9/22; C12N 15/111; A61K 2239/13; A61K 2239/21; A61K 2239/22; A61K 2239/38; C07K 2317/31; C07K 2317/622; C12N 2310/20

(57) ABSTRACT

The invention provides compositions and methods for treating a refractory, relapsed or resistant cancer or a chronic infectious disease. In particular the present invention relates to a modified immune cell with reduced SUV39H1 activity, for use in the treatment of a patient suffering from a refractory, relapsed or resistant cancer or suffering from a chronic infectious disease, wherein the cell expresses one or more engineered antigen-specific receptors that bind an antigen associated with the cancer or the chronic infectious disease

### FIELD OF THE INVENTION

The invention relates to compositions and methods for treating a refractory, relapsed or resistant cancer or a chronic infectious disease.

### BACKGROUND OF THE INVENTION

A variety of cancer therapies, including chemotherapy, antibody therapy, and adoptive cell therapy, are widely available. Yet they encounter hurdles such as cancers that are refractory or resistant to treatment, or cancers that relapse after an initial successful treatment.

Cell immunotherapy has been widely researched and several CAR T-cell therapies are commercially marketed for the treatment of B-cell or lymphoid malignancies. However, despite the impressive rates of initial complete responses observed with current CAR T-cell therapies for B-cell malignancies, longer term follow-up has demonstrated that a significant proportion of patients relapse after treatment. In addition, clinically effective treatment of solid tumors and myeloid malignancies has proven more challenging than treatment of lymphoid malignancies.

There remains a need for better or more effective treatments for cancer.

### SUMMARY OF THE INVENTION

The disclosure provides a modified immune cell with reduced SUV39H1 activity, for use in the treatment of a patient suffering from a refractory, relapsed or resistant cancer or suffering from a chronic infectious disease. Optionally, the cell expresses one or more engineered antigen-specific receptors that bind an antigen associated with the cancer or the chronic infectious disease. In related aspects, the disclosure provides for use of such modified immune cells for preparation of a medicament for treating a patient suffering from a refractory, relapsed or resistant cancer or suffering from a chronic infectious disease. In further related aspects, the disclosure provides for methods of treating a patient suffering from a refractory, relapsed or resistant cancer or suffering from a chronic infectious disease by administering a modified immune cell with reduced SUV39H1 activity, optionally comprising one or more engineered antigen-specific receptors that bind an antigen associated with the cancer or the chronic infectious disease.

In some embodiments, when the patient suffers from a cancer, the patient exhibits a cancer relapse or is likely to exhibit a cancer relapse, exhibits cancer metastasis or is likely to exhibit cancer metastasis, has not achieved sustained cancer remission after one or more prior cancer therapies, suffers from a cancer that is resistant or nonresponsive to one or more prior cancer therapies, suffers from a refractory cancer, is likely to exhibit a response to cell therapy that is not durable, is ineligible for immune checkpoint therapy or did not respond to immune checkpoint therapy, is ineligible for treatment with high dose of chemotherapy and/or is ineligible for treatment with high adoptive cell therapy doses.

In some embodiments, the antigen is orphan tyrosine kinase receptor ROR1, tEGFR, Her2, p95HER2, LI-CAM, CD19, CD20, CD22, mesothelin, CEA, Claudin 18.2, hepatitis B surface antigen, anti-folate receptor, a claudin (such as claudin 6 or claudin 18.2), CD23, CD24, CD30, CD33, CD38, CD44, EGFR, EGP-2, EGP-4, CD70, EPHa2, ErbB2, 3, or 4, FcRH5, FBP, fetal acethycholine e receptor, GD2, GD3, HMW-MAA, IL-22R-alpha, IL-13R-alpha2, kdr, kappa light chain, BCMA, Lewis Y, MAGE-A1, mesothelin, MUC1, MUC16, PSCA, NKG2D Ligands, NY-ESO-1, MART-1, gplOO, oncofetal antigen, TAG72, VEGF-R2, carcinoembryonic antigen (CEA), prostate specific antigen (PSMA), estrogen receptor, progesterone receptor, ephrinB2, CD 123, CS-1, c-Met, GD-2, MAGE A3, CE7, or Wilms Tumor 1 (WT-1); or optionally any of the tumor neoantigenic peptides disclosed in Int'l Pat. Pub. No. WO 2021/043804.

In some embodiments, the cancer is a myeloid cancer. In some embodiments, the cancer is a solid tumor. For example, the solid tumor is a cancer affecting an organ, optionally a cancer affecting colon, rectum, skin, endometrium, lung (including non-small cell lung carcinoma), uterus, bones (such as Osteosarcoma, Chondrosarcomas, Ewing's sarcoma, Fibrosarcomas, Giant cell tumors, Adamantinomas, and Chordomas), liver, kidney, esophagus, stomach, bladder, pancreas, cervix, brain (such as Meningiomas, Glioblastomas, Lower-Grade Astrocytomas, Oligodendrocytomas, Pituitary Tumors, Schwannomas, and Metastatic brain cancers), ovary, breast (such as triple negative breast cancer), head and neck region, testis, prostate or the thyroid gland.

In some embodiments, the chronic infectious disease is a chronic viral infection, optionally wherein the immune cell allows long term infection control and/or increased viral clearance. For example, the chronic viral infection is an infection with human immunodeficiency virus (HIV), Cytomegalovirus (CMV), Epstein-Barr virus (EBV), adenovirus, BK polyomavirus, hepatitis virus, such as hepatitis B, hepatitis C, hepatitis D, or hepatitis E.

In any of the embodiments, the SUV39H1 activity in the modified immune cell can be inactivated or inhibited. In some embodiments, the cell has been contacted with an exogenous SUV39H1 inhibitor, optionally a nucleic acid inhibitor of SUV39H1. In some embodiments, the exogenous SUV39H1 inhibitor is (a) a dominant negative inhibitor, or (b) an RNAi, shRNA, ribozyme or antisense oligonucleotide complementary to a fragment of the SUV39H1 gene, or (c) an epipolythiodioxopiperazine (ETP) class of SUV39H1 inhibitor.

In some embodiments, one or both SUV39H1 genes in the cell comprises one or more mutations that results in a deleted or non-functional SUV39H1 protein, or a SUV39H1 protein with reduced activity.

In some embodiments, the antigen-specific receptor is a modified TCR, or a chimeric antigen receptor (CAR). In some embodiments, cell comprises at least one antigen-specific receptor having an intracellular signaling domain wherein one or two immunoreceptor tyrosine-based activation motifs (ITAMs) are inactivated, optionally wherein the antigen-specific receptor comprises a single active ITAM domain. For example, the antigen-specific receptor is a chimeric antigen receptor (CAR) comprising: a) an extracellular antigen-binding domain that specifically binds an antigen, b) a transmembrane domain, c) optionally one or more costimulatory domains, and d) an intracellular signaling domain wherein one or two immunoreceptor tyrosine-based activation motifs (ITAMs) are inactivated; optionally wherein the antigen-specific receptor comprises a single active ITAM domain; and optionally wherein the intracellular domain comprises a modified CD3zeta intracellular signaling domain in which ITAM2 and ITAM3 have been inactivated.

In some embodiments, the antigen-specific receptor comprises an extracellular antigen-binding domain which is an scFv; or a single domain antibody; or an antibody heavy chain region (VH) and/or an antibody variable region (VL); or optionally a bispecific or trispecific antigen-binding domain. Optionally, the antigen-specific receptor comprises a transmembrane domain from CD28, CD8 or CD3-zeta, or a fragment thereof. Optionally, the antigen-specific receptor comprises one or more costimulatory domains selected from the group consisting of: 4-1BB (CD137), CD28, CD27, ICOS, OX40 (CD134) and DAP10; DAP12, 2B4, CD40, FCER1G and/or GITR (AITR), or an active fragment thereof.

In some embodiments, the antigen-specific receptor comprises: a) an extracellular antigen-binding domain that specifically binds an antigen, optionally comprising an antibody heavy chain variable region and/or an antibody light chain variable region, and is optionally bispecific or trispecific; b) a transmembrane domain, optionally comprising a fragment of transmembrane domain of alpha, beta or zeta chain of the T-cell receptor, CD28, CD3 epsilon, CD45, CD4, CD5, CD8, CD9, CD16, CD22, CD33, CD37, CD64, CD80, CD86, CD34, CD137, or CD154, NKG2D, OX40, ICOS, 2B4, DAP10, DAP12, CD40; c) optionally one or more co-stimulatory domains from 4-1BB, CD28, ICOS, OX40, DAP10 or DAP12, 2B4, CD40, FCER1G, or an active fragment thereof; and d) an intracellular signaling domain comprising an intracellular signaling domain from CD3zeta, FcR gamma, FcR beta, CD3 gamma, CD3 delta, CD3 epsilon, CDS, CD22, CD79a, CD79b, or CD66d, 2B4, or an active fragment thereof.

In some embodiments, the antigen-specific receptor is a chimeric antigen receptor (CAR) comprising: a) an extracellular antigen-binding domain, optionally an scFv, b) a transmembrane domain, optionally from CD28, CD8 or CD3-zeta, c) one or more co-stimulatory domains, optionally from 4-1 BB, CD28, ICOS, OX40 or DAP10, and d) an intracellular signaling domain from CD3zeta, optionally in which ITAM2 and ITAM3 have been inactivated.

In some embodiments, the antigen-specific receptor is a modified TCR that comprises a heterologous extracellular antigen-binding domain that specifically binds an antigen, optionally comprising an antibody heavy chain variable region and/or an antibody light chain variable region, and is optionally bispecific or trispecific. Optionally, the antigen-specific receptor is a modified TCR that comprises a fragment of an alpha, beta, gamma or epsilon chain and a heterologous extracellular antigen-binding domain that specifically binds an antigen, optionally comprising one or more single domain antibody, or an antibody heavy chain variable region and/or an antibody light chain variable region, optionally an scFv, and is optionally bispecific or trispecific. In some embodiments, modified TCR can be named recombinant HLA-independent (or non-HLA restricted) T cell receptors (referred to as “HI-TCRs”) that bind to an antigen of interest in an HLA-independent manner. HLA independent (modified) TCRs are notably described in International Application No. WO 2019/157454. Such HI-TCRs comprise an antigen binding chain that comprises: (a) an antigen-binding domain that binds to an antigen in an HLA-independent manner, for example, an antigen-binding fragment of an immunoglobulin variable region; and (b) a constant domain that is capable of associating with (and consequently activating) a CD3ζ polypeptide. Because typically TCRs bind antigen in a HLA-dependent manner, the antigen-binding domain that binds in an HLA-independent manner must be heterologous.

The antigen-binding domain or fragment thereof comprises: a single domain antibody (VHH), or a heavy chain variable region (VH) of an antibody and/or a light chain variable region (VL) of an antibody. The constant domain of the TCR is, for example, a native or modified TRAC polypeptide, or a native or modified TRBC polypeptide. The constant domain of the TCR is, for example, a native TCR constant domain (alpha or beta) or fragment thereof. Unlike chimeric antigen receptors, which typically themselves comprise an intracellular signaling domain, the HI-TCR does not directly produce an activating signal; instead, the antigen-binding chain associates with and consequently activates a CD3 polypeptide. The immune cells comprising the recombinant TCR is highly sensitive and typically provide high activity when the targeted antigen is expressed at a low density (typically of less than about 10,000 molecules per cell) on the surface of a cell.

In some embodiments, the antigen-specific receptor is thus a modified TCR that comprises: a) a first antigen-binding chain comprising an antigen-binding fragment of a heavy chain variable region (VH) of an antibody; and b) a second antigen-binding chain comprising an antigen-binding fragment of a light chain variable region (VL) of an antibody; wherein the first and second antigen-binding chains each comprise a TRAC polypeptide or a TRBC polypeptide, optionally wherein at least one of the TRAC polypeptide and the TRBC polypeptide is endogenous, and optionally wherein one or both of the endogenous TRAC and TRBC polypeptides is inactivated.

In some embodiments, a heterologous nucleic acid sequence encoding the antigen-specific receptor or a portion thereof is inserted into the cell genome to express the antigen-specific receptor. In other embodiments, a heterologous nucleic acid sequence outside the cell genome expresses the antigen-specific receptor.

In some embodiments, insertion of the heterologous nucleic acid sequence encoding the antigen-specific receptor or a portion thereof inactivates expression of a native TCR alpha chain and/or a native TCR beta chain. In any of these embodiments, expression of the antigen-specific receptor may be under control of an endogenous promoter of a TCR, optionally an endogenous TRAC promoter.

In any of these embodiments, the immune cell is a T cell, a CD4+ T cell, a CD8+ T cell, a CD4+ and CD8+ T cell, a NK cell, a T regulatory cell, a TN cell, a memory stem T cell (TSCM), a TCM cell, a TEM cell, a monocyte, a dendritic cell, or a macrophage, or a progenitor thereof, optionally a T cell progenitor, a lymphoid progenitor, an NK cell progenitor, a myeloid progenitor, a pluripotent stem cell, an induced pluripotent stem cell (iPSC), a hematopoietic stem cell (HSC), an adipose derived stem cell (ADSC), or a pluripotent stem cell of myeloid or lymphoid lineage. Optionally, the immune cell is a T cell or NK cell, or progenitor thereof.

In any of these embodiments, the modified immune cell may further comprise a second engineered antigen-specific receptor, optionally a modified TCR or CAR, that specifically binds to a second antigen. For example, the modified immune cell comprises a first CAR that binds the antigen and a second CAR that binds a second antigen. As another example, the modified immune cell comprises a CAR that binds the antigen and modified TCR that binds a second antigen. As another example, the modified immune cell comprises a first modified TCR that binds the antigen and a second modified TCR that binds a second antigen. In some embodiments, the modified immune cell comprises three or more engineered antigen-specific receptors.

In any of these embodiments, the modified immune cell may further comprise a heterologous co-stimulatory receptor or molecule. In some embodiments, the co-stimulatory receptor comprises (a) an extracellular domain of a co-stimulatory ligand, optionally from CD80, (b) a transmembrane domain, optionally from CD80, and (c) an intracellular domain of a co-stimulatory molecule, optionally CD28, 4-1BB, OX40, ICOS, DAP10, CD27, CD40, NKGD2, or CD2, preferably 4-1BB.

In some embodiments, the extracellular antigen-binding domain binds, or comprises a VH and/or VL from an antibody that binds, an antigen with a KD affinity of about 1×10⁻⁷ M or less, about 5×10⁻⁸ M or less, about 1×10⁻⁸ M or less, about 5×10⁻⁹ M or less, about 1×10⁻⁹ M or less, about 5×10⁻¹⁰ M or less, about 1×10⁻¹⁰ M or less, about 5×10⁻¹¹ M or less, about 1×10⁻¹¹ M or less, about 5×10⁻¹² M or less, or about 1×10⁻¹² M or less (lower numbers indicating greater binding affinity).

In some embodiments, the antigen has a low density on the cell surface, of less than about 10,000, or less than about 5,000, or less than about 2,000 molecules per cell.

In some embodiments, SUV39H1 expression in the cell is reduced or inhibited by at least about 50%, 60%, 70%, 75%, 80%, 85%, 90% or 95%. In some embodiments, endogenous TCR expression of the cell is reduced by at least about 75%, 80%, 85%, 90% or 95%.

The modified immune cell may be allogeneic or autologous. For example, an HLA-A locus is inactivated in the cell. In some embodiments, HLA class I expression is reduced by at least about 75%, 80%, 85%, 90% or 95%.

In another aspect, the disclosure provides a method of producing the foregoing modified immune cell(s) comprising (a) introducing into the cell (a) a SUV39H1 inhibitor, and (b) (i) a nucleic acid encoding a CAR having one active ITAM or (ii) a heterologous nucleic acid encoding a portion of a modified TCR.

In some embodiments, the method or use comprises administering a CAR T-cell at a dose of less than about 5×10⁷ cells, optionally about 10⁵ to about 10⁷ cells. In some embodiments, the method or use comprises administering a second therapeutic agent, optionally one or more cancer chemotherapeutic agents, cytotoxic agents, cancer vaccines, hormones, anti-angiogens, radiolabelled compounds, immunotherapy, surgery, cryotherapy, and/or radiotherapy, to the subject.

In some embodiments, the second therapeutic agent is an immune checkpoint modulator is administered to the patient. For example, the immune checkpoint modulator is an antibody that specifically binds to, or other inhibitor of, PD1, PDL1, CTLA4, LAG3, BTLA, OX2R, TIM-3, TIGIT, LAIR-1, PGE2 receptor, EP2/4 adenosine receptor, or A2AR, optionally an anti-PD1 or anti-PDL1 antibody.

### BRIEF DESCRIPTION OF THE DRAWINGS

FIG. 1A shows the schematic of the xenogeneic A549 lung adenocarcinoma model in NSG mice infused with 19-BBz CAR T cells (3×10⁵), either Mock (wildtype SUV39H1) or SUV39H1 KO. FIG. 1B shows tumor growth measured by bioluminescence in the days post 19-BBz CAR T cell infusion (black arrow). SUV39H1 KO CAR T cells demonstrate better antitumor response (lower line).

FIG. 2A shows the schematic of the xenogeneic A549 lung adenocarcinoma model in NSG mice infused with 19-28z-1XX CAR T cells (5×10⁴), either Mock (wildtype SUV39H1) or SUV39H1 KO. FIGS. 2B and 2C show the bioluminescence measurements in NSG mice 69 days post Mock 19-28z-1XX CAR T cell infusion (FIG. 2B) or SUV39H1 KO 19-28z-1XX CAR T cells (FIG. 2C). There are 5 out of 7 mice treated with Mock CAR T cells that show tumor growth, in contrast to 2 out of 6 mice treated with SUV39H1 KO CAR T cells.

FIGS. 3A and 3B show tumor growth measured by bioluminescence in the days post 19-BBz Mock (FIG. 3A) or SUV39H1 KO (FIG. 3B) CAR T cell infusion (dose of 9×10⁵ cells, administered at time point indicated by black arrows). Black arrowheads indicate days when flow cytometry analysis was performed on lungs and spleens. FIGS. 3C and 3D show CAR T cell numbers in the lungs and spleens, respectively, of tumor bearing mice at two different time points (Day 8 and 28 post CAR T cell infusion). SUV39H1 KO CAR T cells are more abundant in the spleen after tumor eradication (Day 28). FIGS. 3E and 3F show tumor growth measured by bioluminescence in the days post 19-BBz Mock (FIG. 3E) or SUV39H1 KO (FIG. 3F) CAR T cell infusion (dose of 7.5×10⁵ cells, black arrows). Black arrowheads indicate days when flow cytometry analysis was performed on blood samples. FIG. 3G shows CAR T cell numbers in the blood of tumor bearing mice at two different time points (Day 8 and 63 post CAR T cell infusion). SUV39H1 KO CAR T cells are more abundant in the blood at early time point (Day 8, initial expansion) and particularly at later time points (Day 28, persistence after tumor eradication).

FIG. 4A shows the schematic of the xenogeneic A549 lung adenocarcinoma rechallenge model in NSG mice infused with 19-BBz CAR T cells, either mock or SUV39H1 KO. FIG. 4B shows tumor growth measured by bioluminescence in the days post infusion of 2.5×10⁵ 19-BBz CAR-T cells (administered at time point indicated by black arrow). At this dose, neither Mock nor SUV39H1 KO cells were able to control tumor rechallenges. FIG. 4C shows tumor growth measured by bioluminescence in the days post infusion of 7.5×10⁵ 19-BBz CAR-T cells (black arrow). In both FIGS. 4B and 4C, vertical lines indicate dates of additional tumor cell injections (2×10⁶ A549-CD19 cells each). SUV39H1 KO CAR T cells demonstrate complete control of tumor rechallenges (lower line), while all Mock treated mice have relapses.

FIG. 5A shows the schematic of the xenogeneic A549 lung adenocarcinoma rechallenge model in NSG mice infused with 19-28z-1XX CAR T cells, either mock or SUV39H1 KO. FIG. 5B shows tumor growth measured by bioluminescence in the days post infusion of 2.5×10⁵ Mock (wild type SUV39H1) 19-28z-1XX CAR-T cells (black arrows). FIG. 5C shows tumor growth measured by bioluminescence in the days post infusion of 2.5×10⁵ SUV39H1 KO 19-28z-1XX CAR-T cells (black arrows). In both FIGS. 5B and 5C, vertical lines indicate dates of additional tumor cell injections (2×10⁶ A549-CD19 cells each). SUV39H1 KO CAR T cells demonstrate complete control of tumor rechallenges (no mice have relapses), while 3 out of 7 Mock treated mice have tumor relapses.

FIG. 6A shows the schematic of the xenogeneic model of peripheral central nervous system lymphoma (PCNSL). Briefly, the PCNSL cell line TK was injected intracranially in NSG mice. Six days later, the mice were infused with 9×10⁵ 19-BBz CAR T cells, either mock or SUV39H1 KO. The mice were then followed up for weight loss and other clinical symptoms. FIG. 6B shows the percent mouse survival. Treatment with SUV39H1-KO CAR T cells extends mouse survival compared with treatment with mock CAR T cells.

### DETAILED DESCRIPTION OF THE INVENTION

SUV39H1 is a H3K9-histone methyltransferase that plays a role in silencing memory and stem cell programs during the terminal differentiation of effector CD8+ T cells. Silencing of SUV39H1, in turn, has been shown to enhance long-term memory potential and to increase survival capacity.

Immune cells, particularly T-cells or NK cells, in which SUV39H1 activity has been inactivated or inhibited have been shown herein to have surprisingly good efficacy against difficult to treat cancers, such as solid tumors, or refractory, relapsed or resistant cancer. Cells in which SUV39H1 activity has been inactivated or inhibited, and which also comprise an engineered chimeric antigen receptor (CAR) that has a single active ITAM domain are even more surprisingly efficacious. For immune cells in which expression of SUV39H1 has been inactivated, doses which are ineffective with a conventional CAR surprisingly become therapeutically effective with a CAR having a single active ITAM domain. Thus, the combination of SUV39H1 inactivation and a CAR having a single active ITAM domain (e.g. in which ITAM2 and ITAM3 have been inactivated) results in therapeutic efficacy at relatively lower doses of the cells. The cell therapy is also expected to be surprisingly beneficial for treating chronic infectious diseases.

### Definitions

As used herein, the singular forms “a,” “an,” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of polypeptide, dose, time, temperature, enzymatic activity or other biological activity and the like, is meant to encompass variations of ±10%, ±5%, ±1%, ±0.5%, or even ±0.1% of the specified amount.

The term “antibody” herein is used in the broadest sense and includes polyclonal and monoclonal antibodies, including intact antibodies and functional (antigen-binding) antibody fragments, including fragment antigen binding (Fab) fragments, F(ab′)2 fragments, Fab′ fragments, Fv fragments, recombinant IgG (rlgG) fragments, variable heavy chain (VH) regions capable of specifically binding the antigen, single chain antibody fragments, including single chain variable fragments (scFv), and single domain antibodies (e.g., sdAb, sdFv, nanobody) fragments. The term encompasses recombinant and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgG1, IgG2, IgG3, IgG4, IgM, IgE, IgA, and IgD. In some embodiments the antibody comprises a heavy chain variable region and a light chain variable region.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F (ab′) 2; diabodies; linear antibodies; variable heavy chain (VH) regions, VHH antibodies, single-chain antibody molecules such as scFvs and single-domain VH single antibodies; and multispecific antibodies formed from antibody fragments. In particular embodiments, the antibodies are single-chain antibody fragments comprising a variable heavy chain region and/or a variable light chain region, such as scFvs.

“Inactivation” or “disruption” of a gene refers to a change in the sequence of genomic DNA that causes the gene's expression to be reduced or eliminated, or that cause a non-functional gene product to be expressed. Exemplary methods include gene silencing, knockdown, knockout, and/or gene disruption techniques, such as gene editing through, e.g., induction of breaks and/or homologous recombination. Exemplary of such gene disruptions are insertions, frameshift and missense mutations, deletions, knock-in, and knock-out of the gene or part of the gene, including deletions of the entire gene. Such disruptions can occur in the coding region, e.g., in one or more exons, resulting in the inability to produce a full-length product, functional product, or any product, such as by insertion of a stop codon. Such disruptions may also occur by disruptions in the promoter or enhancer or other region affecting activation of transcription, so as to prevent transcription of the gene. Gene disruptions include gene targeting, including targeted gene inactivation by homologous recombination. Inactivation of a gene can decrease the activity of the expressed product by at least 20%, 30%, 40%, 50%, 60%, 70%, 80%, 90%, 95% or 99% or more as compared to the activity or expression levels of wildtype which is not inactivated.

As used herein, “inhibition” of a gene product refers to a decrease of its activity and/or gene expression of at least 20%, 30%, 40%, 50%, 60%, 70%, 80%, 90%, 95% or 99% or more as compared to the activity or expression levels of wildtype which is not inhibited or repressed.

As used herein, “express” or “expression” means that a gene sequence is transcribed, and optionally, translated. If the gene expresses a noncoding RNA, expression will typically result in an RNA after transcription and, optionally, splicing. If the gene is a coding sequence, expression will typically result in production of a polypeptide after transcription and translation.

As used herein, “expression control sequence” refers to a nucleotide sequence that influences the transcription, RNA processing, RNA stability, or translation of the associated nucleotide sequence. Examples include, but are not limited to, promoters, enhancers, introns, translation leader sequences, polyadenylation signal sequences, transcription initiators and transcriptional and/or translational termination region (i.e., termination region).

As used herein, “fragment” means a portion of a referenced sequence (polynucleotide or polypeptide) that has a length at least 60%, 65%, 70%, 75%, 80%, 85%, 90%, 95%, 96%, 97%, 98%, or 99% of the full-length sequence.

As used herein, “heterologous” refers to a polynucleotide or polypeptide that comprises sequences that are not found in the same relationship to each other in nature. For example, the heterologous sequence either originates from another species, or is from the same species or organism but is modified from either its original form or the form primarily expressed in the cell. Thus, a heterologous polynucleotide includes a nucleotide sequence derived from and inserted into the same natural, original cell type, but which is present in a non-natural state, e.g., a different copy number, and/or under the control of different regulatory sequences than that found in nature and/or located in a different position (adjacent to a different nucleotide sequence) than where it was originally located.

As used herein, “nucleic acid,” “nucleotide sequence,” and “oligonucleotide” or “polynucleotide” are used interchangeably and encompass both RNA and DNA, including cDNA, genomic DNA, mRNA, synthetic (e.g., chemically synthesized) DNA or RNA and chimeras of RNA and DNA. The term polynucleotide, nucleotide sequence, or nucleic acid refers to a chain of nucleotides without regard to length of the chain. The nucleic acid can be double-stranded or single-stranded. Where single-stranded, the nucleic acid can be a sense strand or an antisense strand. The nucleic acid can be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases. The present disclosure further provides nucleic acid inhibitors that are complementary to a nucleic acid, nucleotide sequence, or polynucleotide described herein. Modified bases (modified nucleobases), such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made.

As used herein, “operably linked” means that an element, such as an expression control sequence, is configured so as to perform its usual function upon a nucleotide sequence of interest. For example, a promoter operably linked to a nucleotide sequence of interest is capable of effecting expression of the nucleotide sequence of interest. The expression control sequences need not be contiguous with the nucleotide sequence of interest, so long as they function to direct the expression thereof.

As used herein, the expression “percentage of identity” between two sequences, means the percentage of identical bases or amino acids between the two sequences to be compared, obtained with the best alignment of said sequences, this percentage being purely statistical and the differences between these two sequences being randomly spread over the two sequences. A base is considered complementary if it hybridizes under normal conditions. For example, a modified nucleobase can be aligned in a manner like the base whose hybridization pattern it mimics. As used herein, “best alignment” or “optimal alignment”, means the alignment for which the determined percentage of identity (see above) is the highest. Sequence comparison between two nucleic acid sequences (also referenced herein as nucleotide sequence or nucleobase sequence) is usually realized by comparing these sequences that have been previously aligned according to the best alignment. This comparison is realized on segments of comparison in order to identify and compared the local regions of similarity. The best sequence alignment to perform comparison can be realized, besides manually, by using the global homology algorithm developed by SMITH and WATERMAN (Ad. App. Math., vol. 2, p: 482, 1981), by using the local homology algorithm developed by NEEDLEMAN and WUNSCH (J. Mol. Biol, vol. 48, p: 443, 1970), by using the method of similarities developed by PEARSON and LIPMAN (Proc. Natl. Acad. Sci. USA, vol. 85, p: 2444, 1988), by using computer software using such algorithms (GAP, BESTFIT, BLAST P, BLAST N, FASTA, TFASTA in the Wisconsin Genetics software Package, Genetics Computer Group, 575 Science Dr., Madison, WI, USA), by using the MUSCLE multiple alignment algorithms (Edgar, Robert C, Nucleic Acids Research, vol. 32, p: 1792, 2004). To get the best local alignment, one can preferably use BLAST software. The identity percentage between two sequences is determined by comparing these two sequences optimally aligned, the sequences being able to comprise additions or deletions with respect to the reference sequence in order to get the optimal alignment between these two sequences. The percentage of identity is calculated by determining the number of identical positions between these two sequences and dividing this number by the total number of compared positions, and by multiplying the result obtained by 100 to get the percentage of identity between these two sequences.

As used herein, “treatment”, or “treating” involves application of cells of the disclosure or a composition comprising the cells to a patient in need thereof with the purpose to cure, heal, alleviate, relieve, alter, remedy, ameliorate, improve or affect the disease such as cancer, or any symptom of the disease (e.g., cancer or infectious disease). In particular, the terms “treat” or treatment” refers to reducing or alleviating at least one adverse clinical symptom associated with the disease. With reference to cancer treatment, the term “treat” or “treatment” also refers to slowing or reversing the progression of neoplastic uncontrolled cell multiplication, i.e. shrinking existing tumors and/or halting tumor growth. The term “treat” or “treatment” also refers to inducing apoptosis in cancer or tumor cells in the subject.

As used herein, “variant” means a sequence (polynucleotide or polypeptide) that has mutations (deletion, substitution or insertion) that is at least 60%, 65%, 70%, 75%, 80%, 85%, 90%, 95%, 96%, 97%, 98%, or 99% identical to a referenced sequence over its full length or over a region of at least about 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, or 1100 nucleotides or amino acids. With respect to a polynucleotide sequence, variant also encompasses a polynucleotide that hybridizes under stringent conditions to the referenced sequence or complement thereof.

By “modulate” is meant positively or negatively alter. Exemplary modulations include a about 1%, about 2%, about 5%, about 10%, about 25%, about 50%, about 75%, or about 100% change.

By “increase” is meant to alter positively by at least about 5%. An alteration may be by about 5%, about 10%, about 25%, about 30%, about 50%, about 75%, about 100% or more.

By “reduce” is meant to alter negatively by at least about 5%. An alteration may be by about 5%, about 10%, about 25%, about 30%, about 50%, about 75%, or even by about 100%.

By “effective amount” is meant an amount sufficient to have a therapeutic effect. In certain embodiments, an “effective amount” is an amount sufficient to arrest, ameliorate, or inhibit the continued proliferation, growth, or metastasis (e.g., invasion, or migration) of a neoplasia.

By “isolated cell” is meant a cell that is separated from the molecular and/or cellular components that naturally accompany the cell.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

The term “antigen-binding domain” as used herein refers to a domain capable of specifically binding a particular antigenic determinant or set of antigenic determinants present on a cell.

“Linker”, as used herein, shall mean a functional group (e.g., chemical or polypeptide) that covalently attaches two or more polypeptides or nucleic acids so that they are connected to one another. As used herein, a “peptide linker” refers to one or more amino acids used to couple two proteins together (e.g., to couple VH and VL domains). In certain embodiments, the linker comprises a sequence set forth in GGGGSGGGGSGGGGS [SEQ ID NO: 19]

As used herein, a “vector” is any nucleic acid molecule for the transfer into or expression of a nucleic acid in a cell. The term “vector” includes both viral and nonviral (e.g., plasmid) nucleic acid molecules for introducing a nucleic acid into a cell in vitro, ex vivo, and/or in vivo. Vectors may include expression control sequences, restriction sites, and/or selectable markers. A “recombinant” vector refers to a vector that comprises one or more heterologous nucleotide sequences. The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

The term “lentiviral vector” refers to a vector derived from at least a portion of a lentivirus genome, including especially a self-inactivating lentiviral vector as provided in Milone et al., Mol. Ther. 17 (8): 1453-1464 (2009). Other examples of lentivirus vectors that may be used in the clinic, include but are not limited to, e.g., the LENTIVECTOR® gene delivery technology from Oxford BioMedica, the LENTIMAX™ vector system from Lentigen and the like. Nonclinical types of lentiviral vectors are also available and would be known to one skilled in the art.

The term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

By “signal sequence” or “leader sequence” is meant a peptide sequence (e.g., 5, 10, 15, 20, 25 or 30 amino acids) present at the N-terminus of newly synthesized proteins that directs their entry to the secretory pathway. Exemplary leader sequences include, but is not limited to, the IL-2 signal sequence: MYRMQLLSCIALSLALVINS [SEQ ID NO: 20] (human), MY SMQLASC VTLTLVLLVN S [SEQ ID NO: 21] (mouse); the kappa leader sequence: METP AQLLFLLLLWLPDTT G [SEQ ID NO: 22] (human), METDTLLLW VLLLW VPGS T G [SEQ ID NO: 23] (mouse); the CD8 leader sequence: M ALP VT ALLLPL ALLLH A ARP [SEQ ID NO: 24] (human); the truncated human CD8 signal peptide: M ALP VT ALLLPL ALLLH A [SEQ ID NO: 25] (human); the albumin signal sequence: MKWVTFISLLFSSAYS [SEQ ID NO: 26] (human); and the prolactin signal sequence: MD SKGS SQKGSRLLLLLVV SNLLLCQGVV S [SEQ ID NO: 27] (human). By “soluble” is meant a polypeptide that is freely diffusible in an aqueous environment (e.g., not membrane bound).

By “specifically binds” is meant a polypeptide or fragment thereof that recognizes and binds to a biological molecule of interest (e.g., a polypeptide), but which does not substantially recognize and bind other molecules in a sample, for example, a biological sample, which naturally includes a presently disclosed polypeptide.

The term “tumor antigen” as used herein refers to an antigen (e.g., a polypeptide) that is uniquely or differentially expressed on a tumor cell compared to a normal or non-IS neoplastic cell. In certain embodiments, a tumor antigen includes any polypeptide expressed by a tumor that is capable of activating or inducing an immune response via an antigen recognizing receptor (e.g., CD19, MUC-16) or capable of suppressing an immune response via receptor-ligand binding (e.g., CD47, PD-L1/L2, B7.1/2).

“Relapsed” or a “relapse” as used herein refers to the reappearance of a disease (e.g., cancer) or the signs and symptoms of a disease such as cancer after a period of improvement or responsiveness, e.g., after prior treatment of a therapy, e.g., cancer therapy. For example, the period of responsiveness may involve the level of cancer cells falling below a certain threshold, e.g., below 20%, 15%, 10%, 5%, 4%, 3%, 2%, or 1%. The reappearance may involve the level of cancer cells rising above a certain threshold, e.g., above 20%, 15%, 10%, 5%, 4%, 3%, 2%, or 1%.

### Inhibitors of SUV39H1 Expression or Activity.

Human SUV39H1 sequence is provided in UniProt Accession No. 043463. The gene locus of SUV39H1 is located on the X chromosome (position p11.23, 48695554-48709016, in the GRCh38.p13 assembly). One exemplary human gene sequence is SEQ ID NO: 15, and one exemplary human protein sequence is SEQ ID NO: 16, but it is understood that polymorphisms or variants with different sequences exist in various subjects' genomes. In some embodiments herein, SUV39H1 gene expression is reduced or eliminated by inactivation or disruption of one or both SUV39H1 genes. In other embodiments herein, SUV39H1 activity, as opposed to expression, is repressed. Known methods for gene repression include gene silencing, knockdown, knockout, and/or gene disruption techniques, such as gene editing.

SUV39H1 inhibitors can be gene editing agents; small molecule inhibitors; dominant negative inhibitors; antibody derivatives such as intrabodies, nanobodies or affibodies that typically block or inhibit Suv39h1 expression or activity; aptamers that typically block or inhibit Suv39h1 expression or activity; nucleic acid molecules that block or inhibit transcription or translation, such as antisense oligonucleotides complementary to Suv39h1; RNA interfering agents (such as a small interfering RNA (siRNA), small hairpin RNA (shRNA), microRNA (miRNA), or a piwiRNA (piRNA); ribozymes and combinations thereof.

Examples of inhibitors include gene editing techniques which result in targeted gene inactivation or disruption, e.g., by induction of breaks and/or homologous recombination. Gene editing agents include exogenous nuclease systems, or exogenous nucleic acid encoding the nuclease system. Examples include CRISPR-Cas systems comprising a) an engineered, non-naturally occurring Clustered Regularly Interspaced Short Palindromic Repeats (CRISPR) guide RNA that hybridizes with Suv39h1 genomic nucleic acid sequence and/or b) a nucleotide sequence encoding a CRISPR protein (e.g., a Type-II Cas9 protein or a Cpf1/Cas12 protein). The gene editing agent may also be a Zinc finger protein (ZFN) or a TAL protein.

In some embodiments Cas9 CRISPR guide that hybridizes with Suv39h1 genomic nucleic acid sequence having a sequence selected from the table below can be used according to the present invention.

| gRNA | SUV39H1 | Targeting | | | |
|---|---|---|---|---|---|
| name | Exon | sequence | PAM | Position | Strand |
| gRNA- | 2 | GGTTCCTCTT | AGG | 48698980 | − |
| SUV-1 | | AGAGATACCG | | | |
| gRNA- | 3 | GATATCCACG | AGG | 48700107 | − |
| SUV-2 | | CCATTTCACC | | | |
| gRNA- | 3 | TTGATGTACA | | 48700396 | − |
| SUV-3 | | CGAAGGCCCG | | | |
| | | CGG | | | |
| gRNA- | 3 | GCATCTTCCG | GGG | 48700661 | + |
| SUV-4 | | CACGGATGAT | | | |
| gRNA- | 3 | TCGCAAGAAC | | 48700716 | + |
| SUV-5 | | AGCTTCGTCA | | | |
| | | TGG | | | |

Dominant negative inhibitors include mutant proteins without catalytic activity. Examples include SUV39H1 protein carrying an inactivating H324K point mutation within the HMT domain; or SUV39HIΔSET, a deletion construct that lacks the catalytic domain. See Carbone et al. (2006), Mol. Cell. Bio., 26 (4), 1288-1296.

Yet other inhibitors of SUV39H1 activity include small molecule inhibitors, for example, in the epipolythiodioxopiperazine (ETP) class.

The term “small organic molecule” refers to a molecule of a size comparable to those organic molecules generally used in pharmaceuticals. The term excludes biological macro molecules (e. g., proteins, nucleic acids, etc.). Preferred small organic molecules range in size up to about 5000 Da, more preferably up to 2000 Da, and most preferably up to about 1000 Da.

In one embodiment, the inhibitor of H3K9-histone methyltransferase SUV39H1 is chaetocin (CAS 28097 March 2) as described by Greiner D, Bonaldi T, Eskeland R, Roemer E, Imhof A. “Identification of a specific inhibitor of the histone methyltransferase SU (VAR) 3-9”. Nat Chem Biol. 2005 August; 1 (3): 143-5.; Weber, H. P., et al, “The molecular structure and absolute configuration of chaetocin”, Acta Cryst, B28, 2945-2951 (1972); Udagawa, S., et al, “The production of chaetoglobosins, sterigmatocystin, O-methylsterigmatocystin, and chaetocin by Chaetomium spp. and related fungi”, Can. J. microbiol, 25, 170-177 (1979); and Gardiner, D. M., et al, “The epipolythiodioxopiperazine (ETP) class of fungal toxins: distribution, mode of action, functions and biosynthesis”, Microbiol, 151, 1021-1032 (2005). For example, chaetocin is commercially available from Sigma Aldrich.

An inhibitor of SUV39H1 can also be ETP69 (Rac-(3S,6S,7S,8aS)-6-(benzo[d][1,3]dioxol-5-yl)-2,3,7-trimethyl-1,4-dioxohexahydro-6H-3,8a-epidithiopyrrolo[1,2-alpyrazine-7-carbonitrile), a racemic analog of the epidithiodiketopiperazine alkaloid chaetocin A (see WO2014066435 but see also Baumann M, Dieskau A P, Loertscher B M, et al. Tricyclic Analogues of Epidithiodioxopiperazine Alkaloids with Promising In Vitro and In Vivo Antitumor Activity. Chemical science (Royal Society of Chemistry: 2010). 2015; 6:4451-4457, and Snigdha S, Prieto G A, Petrosyan A, et al. H3K9me3 Inhibition Improves Memory, Promotes Spine Formation, and Increases BDNF Levels in the Aged Hippocampus. The Journal of Neuroscience. 2016; 36 (12): 3611-3622).

The inhibiting activity of a compound may be determined using various methods as described in Greiner D. Et al. Nat Chem Biol. 2005 August; l (3): 143-5 or Eskeland, R. et al. Biochemistry 43, 3740-3749 (2004).

Inhibitors include intrabodies, which are antibodies that bind intracellularly to their antigen after being produced in the same cell. For a review, for example, see Marschall et al., MAbs. 2015; 7 (6): 1010-35. Another intrabody format particularly suitable for cytoplasmic expression are single domain antibodies (also called nanobodies) derived from camels or consisting of one human VH domain or human VL domain. These single domain antibodies often have advantageous properties, e.g., high stability; good solubility; ease of library cloning and selection; high expression yield in E. coli and yeast.

Inhibitors include aptamers that inhibit or block SUV39H1 expression or activity. Aptamers are oligonucleotide (DNA or RNA) or oligopeptide sequences with the capacity to recognize virtually any class of target molecules with high affinity and specificity. Oligonucleotide aptamers may be isolated through Systematic Evolution of Ligands by Exponential enrichment (SELEX) of a random sequence library, as described in Tuerk C. and Gold L., 1990 or as reviewed in Jayasena S. D., 1999. Peptide aptamers may consist of a conformationally constrained antibody variable region displayed by a platform protein, such as E. coli Thioredoxin A that are selected from combinatorial libraries by two hybrid methods (Colas P, Cohen B, Jessen T, Grishina I, McCoy J, Brent R. “Genetic selection of peptide aptamers that recognize and inhibit cyclin-dependent kinase 2”. Nature. 1996 Apr. 11; 380 (6574): 548-50).

Inhibitors include affibody molecules. Affibody are small proteins engineered to bind to a large number of target proteins or peptides with high affinity, imitating monoclonal antibodies, and are therefore a member of the family of antibody mimetics (see for review Löfblom J, Feldwisch J, Tolmachev V, Carlsson J, Ståhl S, Frejd F Y. Affibody molecules: engineered proteins for therapeutic, diagnostic and biotechnological applications. FEBS Lett. 2010 Jun. 18; 584 (12): 2670-80). Affibody molecules are based on an engineered variant (the Z domain) of the B-domain in the immunoglobulin-binding regions of staphylococcal protein A, with specific binding for theoretically any given target.

Inhibitors include “nucleic acid inhibitors”, which utilize hybridization or complementary to target gene repression. Examples include antisense oligonucleotides, RNAi, siRNA, shRNA, and/or ribozymes, all of which are well known in the art. For example, the nucleic acid inhibitors can be fully complementary to SUV39H1 mRNA over a stretch of about 12, 15, or 20 contiguous bases; or the inhibitors can be at least 90% complementary, or at least 80% complementary, or at least 70% complementary over the 12, 15 or 20 contiguous bases.

Typically, after transfection of siRNAs or expression of shRNAs, RNAi triggers the degradation of target RNA molecules through direct complementarity, mediated by the RNA-induced silencing complex. An alternative to RNAi for the degradation of lncRNAs are ASOs. ASOs are 15-20-nucleotide single-stranded DNA oligomers that are typically chemically modified to increase the efficacy of knockdown and decrease in vivo toxicity. In particular, the 2′-MOE and LNA gapmer modifications have been shown to increase affinity toward target RNA transcripts and endow resistance to nucleases, allowing these modified ASOs to have half-lives between days to several weeks in vivo. ASOs hybridize with target RNA transcripts through complementarity and induce RNaseH-mediated degradation of the target transcripts.

RNA chemical modifications include one or more of a modified backbone linkage, a modified sugar moiety, a modified phosphate moiety, a modified nucleobase, or a chemically conjugated moiety. Such chemical modifications can be present throughout the polynucleotide, or in alternating patterns. Chemical modifications can be present at the 5′ end or 3′ end or both, e.g. the 5-10 bases at the 5′ and/or 3′ end comprise one or more of a modified backbone linkage, a modified sugar moiety, a modified phosphate moiety, a modified nucleobase, or a chemically conjugated moiety.

Such nucleic acid inhibitors can be delivered directly, as heterologous RNA or as heterologous polynucleotides, e.g., chemically modified polynucleotides that have been modified to increase their serum half-life and/or affinity. Alternatively, such nucleic acid inhibitors can be expressed ectopically from DNA or vectors. In yet another alternative, endogenous expression of such nucleic acid inhibitors can be upregulated, e.g., by inserting expression control sequences, such as constitutive, inducible, strong or tissue-specific promoters.

Inhibition of SUV39H1 in the cell can be achieved before or after injection in the targeted patient. In some embodiment, inhibition as described herein is performed in vivo after administration of the cell to the subject. In some embodiments, a SUV39H1 inhibitor as herein defined can be included in the composition containing the cell. SUV39H1 inhibitors may also be administered separately before, concomitantly of after administration of the cell(s) to the subject.

In some embodiments, inhibition of SUV39H1 according to the invention may be achieved with incubation of a cell with a composition containing at least one SUV39H1 inhibitor, or introduction of at least SUV39H1 inhibitor into the cell, or introduction of one or more nucleic acids encoding a SUV39H1 inhibitor into the cell, as previously described. The SUV39H1 inhibitor is included during the proliferation or expansion of the modified immune cells in vitro.

suitable SUV39H1 inhibitors include, for example, agents that hybridize or bind to the SUV39H1 gene or its regulatory elements, such as aptamers that block or inhibit SUV39H1 expression or activity; nucleic acid molecules that block transcription or translation, such as antisense molecules complementary to SUV39H1; RNA interfering agents (such as a small interfering RNA (siRNA), small hairpin RNA (shRNA), Long noncoding RNAs (lncRNAs), microRNA (miRNA), or a piwiRNA (piRNA); ribozymes and combination thereof. It has been shown that antisense lncRNA, AF196970.3 has silencing function against SUV39H1 in human cells, inhibiting SUV39H1 expression and thus lowering levels of SUV39H1 protein in the cell. AF196970.3 has an expression pattern very similar to the expression pattern of SUV39H1. It is detected in many different cell types, with endothelial cells, fibroblasts and myocytes showing the highest levels, similar to SUV39H1. The gene locus of SUV39H1 is on the X chromosome (position p11,23, 48695554-48709016, in the GRCh38.p13 assembly), At the same locus, in anti-parallel orientation, the non-annotated gene ENSG00000232828 is located at positions 48698963-48737163. Both genes have annotated promoter regions. SEQ ID NO: 55: lncRNA AF196970.3 RNA sequence (CGAGGCAGGGCUUUGGCUACUGGAGAUCGUAGGUUCGAAUCCCGUCUGGGAAGU UCAACUUGUGCACCUGUAAAAGAAGCUGGCAUUAUUGGCUUGUACUCAAGGGCU GGCACAGAGUGUGUCGGGGUGCGGACGCCCCAGCCACGCCCAUCAUCCGUGCGGA AGAUGCAGAGGUCAUAUCGGAUACCCUUCUGUACCACACGAUUUGGGCAGUCAUA GCCGCAGCGGCAGCGGGAGUUGCACUCGUAGAUGGGCAGCCCGGCUCGAAGCCGC ACCUGGCCCUGGUCAUUGUAGGCAAACUUGUGCAGUGACGCCCCCGGGCAGCAGC CUCCAGUGGGUGCCCACAGACAGUCCUGGCACUCGCAGCCCACAGCCACCUGGUU GAGGGUGAUGCCCUCACCAACACGGUACUCAUUGAUGUACACGAAGGCCCGCGGA GGGCCGUCCAGGUCCACCUCAUUCUCUACAGUGAUGCGUCCCAGAUGGCUGCGCU UGGCAUUGAGCUCCUGCUCCCAGCGACGGAGCGCCCGCCUCUGCUUGGCCUUCUG CACCAGGUAGUUGGCCAAGCUUGGGUCCAGGUGCCGGGGGGUCUUUGACCGGUGG UGCCGCCGGAGCAGCUCCCUUUCUAAGUCCUUGUGGAACUGCUUGAGGAUACGCA CACACUUGAGAUUCUGCCGUGGCUCCCAGGUGCUCUCUGAGUCUGGAUAUCCACG CCAUUUCACCAGGGUCAAAGGAGAAAAUUCCCUUUGGAAACAGAUGUGGGCAGU UGGGGACAAGAGGGCAGGACACUAACUUCCUUGUGACCUGUCCCCUCCCAGAGCA UGGUCACCCCAGACUCACGCGGAUCUUCUUGUAAUCGCACAGGUACUCGACUUCA AAGUCAUAGAGGUUCCUCUUAGAGAUACCGAGGGCAGGGCAGGAGAGC) is the predicted RNA sequence expressed from ENSG00000232828, after transcription and splicing. SEQ ID NO: 1 is a 925 base sequence that includes three exons, AF196970.3 exon 1 is 125 bases in length and does not have significant complementarity to the SUV39H1 gene. AF196970.3 exon 2 is 600 bases in length and is anti-parallel to a substantial portion of exon 3 of the SUV39HI gene with 100% similarity. AF196970.3 exon 3 is 200 bases in length and is anti-parallel to a portion (42.5% similarity) of exon 2 of the SUV39G1 gene, as well as a portion of the adjacent intron. Inhibitory polynucleotides of the disclosure, for use in the cells and methods of the disclosure, include AF196970.3 (SEQ ID NO: 1) or fragments or variants thereof.

### Expression of Proteins or RNA

Means and vectors for expressing polypeptides or polynucleotides such as RNA are well known in the art and commercially available. Known vectors include viral vectors and pseudotyped viral vectors, such as retrovirus (e.g., moloney murine leukemia virus, harvey murine sarcoma virus, murine mammary tumor virus, and rous sarcoma virus), lentivirus, adenovirus, adeno-associated virus (AAV), alphavirus, vaccinia virus, poxvirus, SV40-type viruses, polyoma viruses, Epstein-Barr viruses, herpes simplex virus, papilloma virus, polio virus, foamivirus, or Semliki Forest virus vectors; or transposase systems, such as Sleeping Beauty transposase vectors. Non-viral systems for delivery of naked plasmids to cells include lipofection, nucleofection, microinjection, biolistics, virosomes, lipids, cationic lipid complexes, liposomes, immunoliposomes, nanoparticle, gold particle, or polymer complex, poly-lysine conjugates, synthetic polyamino polymers, other agent-enhanced uptake of DNA, and artificial viral envelopes or virions.

For expressing short noncoding RNAs, such as shRNA, polymerase III promoters are commonly used. Examples include U6, H1, or 7SK promoters.

For expressing long RNAs, such as mRNAs or long noncoding RNAs, polymerase II promoters are commonly used. Examples include CMV, EF-1a, hPGK and RPBSA. CAG promoters have been used to overexpress lncRNA. Yin et al Cell Stem Cell. 2015 May 7; 16 (5): 504-16. Inducible promoters that are driven by signals from activated T cells include nuclear factor of activated T cells (NFAT) promoter. Other promoters for T cell expression of RNA include CIFT chimeric promoter (containing portions of cytomegalovirus (CMV) enhancer, core interferon gamma (IFN-γ) promoter, and a T-lymphotropic virus long terminal repeat sequence (TLTR)), endogenous TRAC promoter or TRBC promoter. Inducible, constitutive, or tissue-specific promoters are contemplated.

In some embodiments, dsRNA such as RNAi is produced in the cell by a vector comprising an expression control sequence operatively linked to a nucleotide sequence that expresses (is a template for one or both strands of) the dsRNA. In further embodiments, a promoter can flank either end of the template nucleotide sequence, wherein the promoters drive expression of each individual DNA strand, thereby generating two complementary (or substantially complementary) RNAs that hybridize and form the dsRNA. In other embodiments, dsRNA is produced in the cell by a vector that expresses an shRNA that is processed to form an interfering dsRNA.

### Delivery of Nucleic Acids or Polynucleotides, Including Vectors

Art-recognized techniques for introducing foreign nucleic acids (e.g., DNA and RNA) into a host cell, include calcium phosphate or calcium chloride co-precipitation, DEAE-dextran-mediated transfection, lipofection, electroporation, microinjection, DNA-loaded liposomes, lipofectamine-DNA complexes, cell sonication, gene bombardment using high velocity microprojectiles, biolistics, and viral-mediated transfection. Compositions comprising the nucleic acids may also comprise transfection facilitating agents, which include surface active agents, quinone analogs, vesicles such as squalene and squalene, hyaluronic acid, lipids, liposomes, lecithin liposomes, calcium ions, viral proteins, polyanions, polycations, including poly-L-glutamate, or nanoparticles, gold particles, or other known agents. Delivery vehicles include a liposome, lipid-containing complex, nanoparticle, gold particle, or polymer complex.

Lipid materials have been used to create lipid nanoparticles (LNPs) based on ionizable cationic lipids, which exhibit a cationic charge in the lowered pH of late endosomes to induce endosomal escape, because of the tertiary amines in their structure. These LNPs have been used, for example, to deliver RNA interference (RNAi) components, as well as genetic constructs or CRISPR-Cas systems. See, such as, Wilbie et al., Acc Chem Res.; 52 (6): 1555-1564, 2019. Wang et al., Proc Natl Acad Sci US A.; 113 (11): 2868-2873, 2016 describe use of biodegradable cationic LNPs. Chang et al., Acc. Chem. Res., 52, 665-675, 2019 describe use of ionizable lipid along with cholesterol, DSPC, and a PEGylated lipid to create LNPs.

Polymer based particles can be used for genetic construct delivery in a similar manner as lipids. Numerous materials have been used for delivery of nucleic acids. For example, cationic polymers such as polyethylenimine (PEI) can be complexed to nucleic acids and can induce endosomal uptake and release, similarly to cationic lipids. Dendrimeric structures of poly(amido-amine) (PAMAM) can also be used for transfection. These particles consist of a core from which the polymer branches. They exhibit cationic primary amines on their surface, which can complex to nucleic acids. Networks based on zinc to aid cross-linking of imidazole have been used as delivery methods, relying on the low pH of late endosomes which, upon uptake, results in cationic charges due to dissolution of the zeolitic imidazole frameworks (ZIF), after which the components are released into the cytosol. Colloidal gold nanoparticles have also been used. See Wilbie et al., supra.

### Production and Chemical Modification of Nucleic Acid Inhibitors

In vitro transcribed, chemically synthesized, or partially chemically synthesized RNAs can be delivered. For example, an RNA may be in vitro transcribed and chemically linked to chemically synthesized RNA at the 5′ end and/or 3′ end. Direct injection or transfection of in vitro-transcribed lncRNAs has been performed to demonstrate lncRNA function. Ulitsky et al. (2011) Cell, 147 (7): 1537-1550.

Chemical modifications to oligonucleotides (also referred to as polynucleotides) can improve their resistance to degradation, thereby increasing half-life, and/or increase affinity for complementary polynucleotides. Modified oligonucleotides (e.g., RNA oligonucleotides) comprise chemical modifications including one or more of a modified backbone linkage, a modified phosphate moiety, a modified sugar moiety, a modified nucleobase, or a chemically conjugated moiety, or any combinations thereof. One, two, three, four, five, 10, 15, 20 or more of the same type of modification, optionally in combination with 1, 2, 3, 4, 5, 10, 15, 20 or more of another type of modification, or patterns of modifications are contemplated. Patterns include alternating modifications throughout the oligonucleotide, such as 2′-fluoro and 2′-methoxy, or end modifications wherein, for example, 1, 2, 3, 4, 5, or more of the bases, sugars, or linkages at the 5′ and/or 3′ end of the oligonucleotide are modified.

Examples of modified backbone linkages include phosphorothioate, phosphothioate (PhTx) group or phosphonoacetate, thiophosphonoacetate, methylphosphonate, boranophosphate, or phosphorodithioate. Other internucleotide bridging modified phosphates may be used, such as methyl phosphonothioates, phosphoromorpholidates, phosphoropiperazidates and phosphoramidates. For example, every one or every other one of the internucleotide bridging phosphate residues can be modified as described.

Examples of modified sugar moieties include deoxyribose, or replacement of the 2′ OH-group by another group. Although the majority of sugar analog alterations are localized to the 2′ position, other sites are amenable to modification, including the 4′ position. Example replacement groups include H, —OR, —R (wherein R can be, such as, alkyl, cycloalkyl, aryl, aralkyl, heteroaryl or sugar), lower alkyl moiety (e.g., C₁-C₄, linear or branched, saturated or unsaturated alkyl, such as methyl, ethyl, ethenyl, propyl, 1-propenyl, 2-propenyl, and isopropyl), halo, —F, —Br, —Cl or —I, —SH, —SR (wherein R can be, such as, alkyl, cycloalkyl, aryl, aralkyl, heteroaryl or sugar),-arabino, F-arabino, amino (wherein amino can be, such as, NH₂; alkylamino, dialkylamino, heterocyclyl, arylamino, diarylamino, heteroarylamino, diheteroarylamino, or amino acid); or cyano (—CN). Specific examples include a 2′-fluoro sugar, 2′-O-methyl sugar, 2′-O-methoxyethyl sugar, or a locked nucleic acid (LNA) nucleotide. Specific examples of modified 2′-sugar include 2′-F or 2′-O-methyl, adenosine (A), 2′-F or 2′-O-methyl, cytidine (C), 2′-F or 2′-O-methyl, uridine (U), 2′-F or 2′-O-methyl, thymidine (T), 2′-F or 2′-O-methyl, guanosine (G), 2′-O-methoxyethyl-5-methyluridine (Teo), 2′-O-methoxyethyladenosine (Aeo), 2′-O-methoxyethyl-5-methylcytidine (m5Ceo), and any combinations thereof. For example, every one or every other one of the nucleotides can be modified as described.

Examples of modified nucleobases include, but are not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3) w, and 2,6-diaminopurine.

Polynucleotides can also be stabilized by complexing to lipids or liposomes. In some embodiments, the liposome comprises 1,2-dioleoyl-sn-glycero-3-phosphatidylcholine (DOPC). In certain embodiments, the lipid particle comprises cholesterol, 1,2-distearoyl-sn-glycero-3-phosphocholine (DSPC), PEG-CDMA or PEG-cDSA, and 1,2-dilinoleyloxy-3-(N,N-dimethyl) aminopropane (DLinDMA).

### Immune Cell

Cells according to the disclosure exhibit modulation, preferably inhibition, of SUV39H1 expression. The cells are typically mammalian cells, or cell lines, e.g., mouse, rat, pig, non-human primate, or preferably human. Such cells include cells derived from the blood, bone marrow, lymph, or lymphoid organs (notably the thymus) and are preferably cells of the immune system (i.e., immune cells), such as cells of the innate or adaptive immunity, e.g., myeloid or lymphoid cells, including monocytes, macrophages, dendritic cells, or lymphocytes, typically T cells and/or NK cells. Immune cells or progenitors thereof preferably also comprise one or more, or two or more, or three or more antigen-specific receptors (CAR and/or TCR) as described herein, and optionally comprise one or more co-stimulatory receptors. Among the antigen-specific receptors according to the disclosure are recombinant modified T cell receptors (TCRs) and components thereof, as well as functional non-TCR antigen-specific receptors, such as chimeric antigen receptors (CAR).

Cells according to the disclosure may also be immune cell progenitors, such as lymphoid progenitors and more preferably T cell progenitors. Examples of T-cell progenitors include pluripotent stem cells (PSC), induced pluripotent stem cells (iPSCs), hematopoietic stem cells (HSC), human embryonic stem cells (ESC), adipose-derived stem cells (ADSC), multipotent progenitor (MPP); lymphoid-primed multipotent progenitor (LMPP); common lymphoid progenitor (CLP); lymphoid progenitor (LP); thymus settling progenitor (TSP); or early thymic progenitor (ETP). Hematopoietic stem and progenitor cells can be obtained, for example, from cord blood, or from peripheral blood, e.g. peripheral blood-derived CD34+ cells after mobilization treatment with granulocyte-colony stimulating factor (G-CSF). T cell progenitors typically express a set of consensus markers including CD44, CD117, CD135, and/or Sca-1.

In some embodiments, the cells include one or more subsets of T cells or other cell types, such as whole T cell populations, CD4+ and/or CD8+ T cells, and subpopulations thereof, such as those defined by function, activation state, maturity, potential for differentiation, expansion, recirculation, localization, and/or persistence capacities, antigen-specificity, type of antigen-specific receptor, presence in a particular organ or compartment, marker or cytokine secretion profile, and/or degree of differentiation. In some embodiments, the cells include myeloid derived cells, such as dendritic cells, monocytes or macrophages.

Among the sub-types and subpopulations of T cells and/or of CD4+ and/or of CD8+ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells. Specifically contemplated herein are TEFF cells with stem/memory properties and higher reconstitution capacity due to the inhibition of SUV39H1, as well as TN cells, TSCM, TCM, TEM cells and combinations thereof. In some embodiments, the cells according to the disclosure are TEFF cells with stem/memory properties and higher reconstitution capacity due to the inhibition of Suv39h1, as well as TN cells, TSCM, TCM, TEM cells and combinations thereof.

In some embodiments, one or more of the T cell populations is enriched for, or depleted of, cells that are positive for or express high levels of one or more particular markers, such as surface markers, or that are negative for or express relatively low levels of one or more markers. In some cases, such markers are those that are absent or expressed at relatively low levels on certain populations of T cells (such as non-memory cells) but are present or expressed at relatively higher levels on certain other populations of T cells (such as memory cells). In one embodiment, the cells (such as the CD8+ cells or the T cells, e.g., CD3+ cells) are enriched for (i.e., positively selected for) cells that are positive or expressing high surface levels of CD117, CD135, CD45RO, CCR7, CD28, CD27, CD44, CD127, and/or CD62L and/or depleted of (e.g., negatively selected for) cells that are positive for or express high surface levels of CD45RA. In some embodiments, cells are enriched for or depleted of cells positive or expressing high surface levels of CD122, CD95, CD25, CD27, and/or IL7-Ra (CD127). In some examples, CD8+ T cells are enriched for cells positive for CD45RO (or negative for CD45RA) and for CD62L. The subset of cells that are CCR7+, CD45RO+, CD27+, CD62L+ cells constitute a central memory cell subset.

For example, according to the disclosure, the cells can include a CD4+ T cell population and/or a CD8+ T cell sub-population, e.g., a sub-population enriched for central memory (TCM) cells. Alternatively, the cells can be other types of lymphocytes, including natural killer (NK) cells, mucosal associated invariant T (MAIT) cells, Innate Lymphoid Cells (ILCs) and B cells.

Cells include primary cells, isolated directly from a biological sample obtained from a subject, and optionally frozen. In some embodiments, the subject is in need of a cell therapy (adoptive cell therapy) and/or is the one who will receive the cell therapy. With reference to a subject to be treated with cell therapy, the cells may be allogeneic and/or autologous. In autologous immune cell therapy, immune cells are collected from the patient, modified as described herein, and returned to the patient. In allogeneic immune cell therapy, immune cells are collected from healthy donors, rather than the patient, modified as described herein, and administered to patients. Typically, these are HLA matched to reduce the likelihood of rejection by the host. The immune cells may also comprise modifications to reduce immunogenicity such as disruption or removal of HLA class I molecules, HLA-A locus, and/or Beta-2 microglobulin (B2M).

With reference to the subject to be treated, the cells of the disclosure may be allogeneic and/or autologous.

In autologous immune cell therapy, immune cells are collected from the patient, modified as described herein, and returned to the patient. In allogeneic immune cell therapy, immune cells are collected from healthy donors, rather than the patient, modified as described herein, and administered to patients. Typically these are HLA matched to reduce the likelihood of rejection by the host. The immune cells may also comprise modifications such as disruption or removal of HLA class I molecules. For example, Torikai et al., Blood. 2013; 122:1341-1349 used ZFNs to knock out the HLA-A locus, while Ren et al., Clin. Cancer Res. 2017; 23:2255-2266 knocked out Beta-2 microglobulin (B2M), which is required for HLA class I expression.

Universal “off the shelf product” immune cells typically comprise modifications designed to reduce graft vs. host disease, such disruption or deletion of endogenous TCR. Because a single gene encodes the alpha chain (TRAC) rather than the two genes encoding the beta chain (TRBC), the TRAC locus is a common target for altering, removing or disrupting endogenous TCR expression.

The samples include tissue samples, from tissues or organ, or fluid samples, such as blood, plasma, serum, cerebrospinal fluid, or synovial fluid. Samples may be taken directly from the subject, or may result from one or more processing steps, such as separation, centrifugation, genetic engineering (for example transduction with viral vector), washing, and/or incubation. Blood or blood-derived samples may be derived from an apheresis or a leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, myeloid derived cells, and/or cells derived therefrom. In some embodiments, the cells are derived from cell lines, e.g., T cell lines. The cells can also be obtained from a xenogeneic source, such as a mouse, a rat, a non-human primate, or a pig. Preferably, the cells are human cells.

### Methods for Producing or Manufacturing an Engineered Immune Cell

Provided herein are methods of producing the cells of the disclosure with modulated SUV39H1 expression. For cells with inhibited SUV39H1 expression, such methods include steps of, for example, (a) introducing into the cell an exogenous SUV39H1 inhibitor, or nucleic acid(s) encoding an exogenous SUV39H1 inhibitor, to disrupt or inactivate or inhibit SUV39H1 activity. In some embodiments, the SUV39H1 gene can be disrupted by introducing a gene editing system to delete all or part of the gene, or to mutate the gene to produce a truncated or non-functional protein.

In some embodiments, the immune cell is also modified to impair expression of a functional endogenous TCR. Because a single gene encodes the alpha chain (TRAC) rather than the two genes encoding the beta chain, the TRAC locus is a typical target for reducing TCR receptor expression. It may thus include steps of, for example, (a) introducing into the cell an exogenous TRAC inhibitor, or nucleic acid(s) encoding an exogenous TRAC inhibitor, to disrupt functional TCR expression. In some embodiments, the TRAC gene can be disrupted by introducing a gene editing system as detailed below to delete all or part of the gene, or to mutate the gene to produce a truncated or non-functional protein.

The method of the invention also include a step (b) of introducing a nucleic acid encoding all or part of an antigen-specific receptor, e.g. introducing a nucleic acid encoding an antibody fragment, and optionally (c) introducing a nucleic acid encoding a chimeric co-stimulatory receptor or ligand. Steps a, b, and c can be performed in any order. In some embodiments steps b and c and performed simultaneously typically when nucleic acid encoding the chimeric receptor and the nucleic acid encoding a chimeric co-stimulatory receptor or ligand are present on the same construct (and typically cloned in the same single vector).

A “gene editing system” as the term is used herein, refers to a system, e.g., one or more molecules, that direct and effect an alteration, e.g., a deletion, of one or more nucleic acids at or near a site of genomic DNA targeted by said system. Gene editing systems are known in the art, and are described more fully below.

Suitable SUV39H1 inhibitors can also include an exogenous nucleic acid comprising a) an engineered, non-naturally occurring Clustered Regularly Interspaced Short Palindromic Repeats (CRISPR) guide RNA that hybridizes with SUV39H1 genomic nucleic acid sequence and/or b) a nucleotide sequence encoding a CRISPR protein (typically a Type-II Cas9 protein but see below for other examples), optionally wherein the cells are transgenic for expressing a Cas9 protein, or an RNP comprising the guide RNA and the CRISPR protein. The agent may also be a Zinc finger protein (ZF) or a TAL protein. The Cas9 protein, TAL protein and/or ZF protein are linked directly or indirectly to a repressor and/or inhibitor, or are linked to a nuclease that confers gene editing activity

Suitable SUV39H1 inhibitors can also include non-functional SUV39H1. In some embodiments, the wildtype SUV39H1 gene is not inactivated, but rather a SUV39H1 inhibitor is expressed in the cell. In some embodiments the inhibitor is a dominant negative SUV39H1 gene that expresses non-functional gene product at a level that inhibits activity of the wildtype SUV39H1. This may comprise overexpression of the dominant negative SUV39H1.

The inactivation of SUV39H1 in the immune cell and the introduction of an antigen-specific receptor that specifically binds to a target antigen can be carried out simultaneously or sequentially in any order.

Inactivation of SUV39H1 in a cell according to the disclosure may also be effected via repression or disruption of the SUV39H1 gene, such as by deletion, e.g., deletion of the entire gene, exon, or region, and/or replacement with an exogenous sequence, and/or by mutation, e.g., frameshift or missense mutation, within the gene, typically within an exon of the gene. In some embodiments, the disruption results in a premature stop codon being incorporated into the gene, such that the SUV39H1 protein is not expressed or is non-functional. The disruption is generally carried out at the DNA level. The disruption generally is permanent, irreversible, or not transient.

In some embodiments, the gene inactivation is achieved using gene editing systems such as a DNA-targeting molecule, such as a DNA-binding protein or DNA-binding nucleic acid, or complex, compound, or composition, containing the same, which specifically binds to or hybridizes to the gene. In some embodiments, the DNA-targeting molecule comprises a DNA-binding domain, e.g., a zinc finger protein (ZFP) DNA-binding domain, a transcription activator-like protein (TAL) or TAL effector (TALE) DNA-binding domain, a clustered regularly interspaced short palindromic repeats (CRISPR) DNA-binding domain, or a DNA-binding domain from a meganuclease.

Zinc finger, TALE, and CRISPR system binding domains can be “engineered” to bind to a predetermined nucleotide sequence.

In some embodiments, the DNA-targeting molecule, complex, or combination contains a DNA-binding molecule and one or more additional domain, such as an effector domain to facilitate the repression or disruption of the gene. For example, in some embodiments, the gene disruption is carried out by fusion proteins that comprise DNA-binding proteins and a heterologous regulatory domain or functional fragment thereof. Typically, the additional domain is a nuclease domain. Thus, in some embodiments, gene disruption is facilitated by gene or genome editing, using engineered proteins, such as nucleases and nuclease-containing complexes or fusion proteins, composed of sequence-specific DNA-binding domains fused to, or complexed with, non-specific DNA-cleavage molecules such as nucleases. These targeted chimeric nucleases or nuclease-containing complexes carry out precise genetic modifications by inducing targeted double-stranded breaks or single-stranded breaks, stimulating the cellular DNA-repair mechanisms, including error-prone nonhomologous end joining (NHEJ) and homology-directed repair (HDR). In some embodiments the nuclease is an endonuclease, such as a zinc finger nuclease (ZFN), TALE nuclease (TALEN), an RNA-guided endonuclease (RGEN), such as a CRISPR-associated (Cas) protein, or a meganuclease. Such systems are well-known in the art (see, for example, U.S. Pat. No. 8,697,359; Sander and Joung (2014) Nat. Biotech. 32:347-355; Hale et al. (2009) Cell 139:945-956; Karginov and Hannon (2010) Mol. Cell 37:7; U.S. Pat. Publ. 2014/0087426 and 2012/0178169; Boch et al. (2011) Nat. Biotech. 29:135-136; Boch et al. (2009) Science 326:1509-1512; Moscou and Bogdanove (2009) Science 326:1501; Weber et al. (2011) PLOS One 6: e19722; Li et al. (2011) Nucl. Acids Res. 39:6315-6325; Zhang et al. (2011) Nat. Biotech. 29:149-153; Miller et al. (2011) Nat. Biotech. 29:143-148; Lin et al. (2014) Nucl. Acids Res. 42: e47). Such genetic strategies can use constitutive expression systems or inducible expression systems according to well-known methods in the art.

### ZFPs and ZFNs; TALs, TALEs, and TALENs

In some embodiments, the DNA-targeting molecule includes a DNA-binding protein such as one or more zinc finger protein (ZFP) or transcription activator-like protein (TAL), fused to an effector protein such as an endonuclease. Examples include ZFNs, TALEs, and TALENs. See Lloyd et al., Frontiers in Immunology, 4 (221), 1-7 (2013).

In some embodiments, the DNA-targeting molecule comprises one or more zinc-finger proteins (ZFPs) or domains thereof that bind to DNA in a sequence-specific manner. A ZFP or domain thereof is a protein or domain within a larger protein, that binds DNA in a sequence-specific manner through one or more zinc fingers regions of amino acid sequence within the binding domain whose structure is stabilized through coordination of a zinc ion. Generally, sequence-specificity of a ZFP may be altered by making amino acid substitutions at the four helix positions (−1, 2, 3 and 6) on a zinc finger recognition helix. Thus, in some embodiments, the ZFP or ZFP-containing molecule is non-naturally occurring, e.g., is engineered to bind to the target site of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416.

In some embodiments, the DNA-targeting molecule is or comprises a zinc-finger DNA binding domain fused to a DNA cleavage domain to form a zinc-finger nuclease (ZFN). In some embodiments, fusion proteins comprise the cleavage domain (or cleavage half-domain) from at least one Type IIS restriction enzyme and one or more zinc finger binding domains, which may or may not be engineered. In some embodiments, the cleavage domain is from the Type IIS restriction endonuclease Fok I. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31,978-31,982.

In some aspects, the ZFNs efficiently generate a double strand break (DSB), for example at a predetermined site in the coding region of the targeted gene (i.e. SUV39H1). Typical targeted gene regions include exons, regions encoding N-terminal regions, first exon, second exon, and promoter or enhancer regions. In some embodiments, transient expression of the ZFNs promotes highly efficient and permanent disruption of the target gene in the engineered cells. In particular, in some embodiments, delivery of the ZFNs results in the permanent disruption of the gene with efficiencies surpassing 50%. Many gene-specific engineered zinc fingers are available commercially. For example, Sangamo Biosciences (Richmond, CA, USA) has developed a platform (CompoZr) for zinc-finger construction in partnership with Sigma-Aldrich (St. Louis, MO, USA), allowing investigators to bypass zinc-finger construction and validation altogether, and provides specifically targeted zinc fingers for thousands of proteins. Gaj et al., Trends in Biotechnology, 2013, 31 (7), 397-405. In some embodiments, commercially available zinc fingers are used or are custom designed. (See, for example, Sigma-Aldrich catalog numbers CSTZFND, CSTZFN, CTI1-1KT, and PZD0020).

In some embodiments, the DNA-targeting molecule comprises a naturally occurring or engineered (non-naturally occurring) transcription activator-like protein (TAL) DNA binding domain, such as in a transcription activator-like protein effector (TALE) protein, See, e.g., U.S. Patent Publication No. 20110301073. In some embodiments, the molecule is a DNA binding endonuclease, such as a TALE-nuclease (TALEN). In some aspects the TALEN is a fusion protein comprising a DNA-binding domain derived from a TALE and a nuclease catalytic domain to cleave a nucleic acid target sequence. In some embodiments, the TALE DNA-binding domain has been engineered to bind a target sequence within genes that encode the target antigen and/or the immunosuppressive molecule. For example, in some aspects, the TALE DNA-binding domain may target CD38 and/or an adenosine receptor, such as A2AR.

In some embodiments, the TALEN recognizes and cleaves the target sequence in the gene. In some aspects, cleavage of the DNA results in double-stranded breaks. In some aspects the breaks stimulate the rate of homologous recombination or non-homologous end joining (NHEJ). Generally, NHEJ is an imperfect repair process that often results in changes to the DNA sequence at the site of the cleavage. In some aspects, repair mechanisms involve rejoining of what remains of the two DNA ends through direct re-ligation (Critchlow and Jackson, Trends Biochem Sci. 1998 October; 23 (10): 394-8) or via the so-called microhomology-mediated end joining. In some embodiments, repair via NHEJ results in small insertions or deletions and can be used to disrupt and thereby repress the gene. In some embodiments, the modification may be a substitution, deletion, or addition of at least one nucleotide. In some aspects, cells in which a cleavage-induced mutagenesis event, i.e. a mutagenesis event consecutive to an NHEJ event, has occurred can be identified and/or selected by well-known methods in the art.

TALE repeats can be assembled to specifically target the SUV39H1 gene. (Gaj et al., Trends in Biotechnology, 2013, 31 (7), 397-405). A library of TALENs targeting 18,740 human protein-coding genes has been constructed (Kim et al., Nature Biotechnology. 31, 251-258 (2013)). Custom-designed TALE arrays are commercially available through Cellectis Bioresearch (Paris, France), Transposagen Biopharmaceuticals (Lexington, KY, USA), and Life Technologies (Grand Island, NY, USA). Specifically, TALENs that target CD38 are commercially available (See Gencopoeia, catalog numbers HTN222870-1, HTN222870-2, and HTN222870-3, available on the World Wide Web at www.genecopoeia.com/product/search/detail.php?prt=26&cid=&key=HTN222870). Exemplary molecules are described, e.g., in U.S. Patent Publication Nos. US 2014/0120622, and 2013/0315884.

In some embodiments the TALENs are introduced as transgenes encoded by one or more plasmid vectors. In some aspects, the plasmid vector can contain a selection marker which provides for identification and/or selection of cells which received said vector.

### RGENs (CRISPR/Cas Systems)

The gene repression can be carried out using one or more DNA-binding nucleic acids, such as disruption via an RNA-guided endonuclease (RGEN), or other form of repression by another RNA-guided effector molecule. For example, in some embodiments, the gene repression can be carried out using clustered regularly interspaced short palindromic repeats (CRISPR) and CRISPR-associated proteins. See Sander and Joung, Nature Biotechnology, 32 (4): 347-355.

In general, “CRISPR system” refers collectively to transcripts and other elements involved in the expression of, or directing the activity of, CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

Typically, the CRISPR/Cas nuclease or CRISPR/Cas nuclease system includes a non-coding RNA molecule (guide) RNA, which sequence-specifically binds to DNA, and a CRISPR protein, with nuclease functionality (e.g., two nuclease domains). One or more elements of a CRISPR system can derive from a type I, type II, or type III CRISPR system, such as a Cas protein (e.g. a Cas nuclease). Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, or a variant thereof. In particular embodiments, the Cas protein is Cas9 nuclease. Preferably, the CRISPR protein is a Cas enzyme such as Cas9. Cas enzymes are well-known in the field; for example, the amino acid sequence of S. pyogenes Cas9 protein may be found in the SwissProt database under accession number Q99ZW2.

In some embodiments, a Cas nuclease and gRNA are introduced into the cell. In some embodiments, the CRISPR system induces DSBs at the target site, followed by disruptions as discussed herein. Common to all these Cas9 or Casç-like)-mediated editing strategies is the induction of double-strand breaks (DSBs) in DNA which are subsequently repaired by either non-homologous end joining (NHEJ) or homology-directed repair (HDR). Incomplete repair of these breakage points may give rise to indels leading to frameshift mutations and subsequent knock-out of one or more targeted genes. When combined with DNA donor template, the same toolkit allows the targeted integration of exogenous DNA inserts at defined genomic loci. Indeed, in general, a CRISPR system is characterized by elements that promote the formation of a CRISPR complex at the site of the target sequence. Typically, in the context of formation of a CRISPR complex, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex. The target sequence may comprise any polynucleotide, such as DNA or RNA polynucleotides. Generally, a sequence or template that may be used for recombination into the targeted locus comprising the target sequences is referred to as an “editing template” or “editing polynucleotide” or “editing sequence”. In some aspects, an exogenous template polynucleotide may be referred to as an editing template. In some aspects, the recombination is homologous recombination. In some embodiments, Cas9 variants, deemed “nickases” can be used to nick a single strand at the target site. Paired nickases can also be used, e.g., to improve specificity, each directed by a pair of different gRNAs targeting sequences. In still other embodiments, catalytically inactive Cas9 can be fused to a heterologous effector domain, such as a transcriptional repressor, to affect gene expression.

In some embodiments; nuclease-inactive platforms including base and prime editors (Biederstädt A, Manzar G S, Daher M. Multiplexed engineering and precision gene editing in cellular immunotherapy. Front Immunol. 2022; 13:1063303. Published 2022 Nov. 22) can be used as they offer a potentially safer route to rewriting genetic sequences and introducing large segments of transgenic DNA without inducing double-strand breaks (DSBs).

Base editing typically refers to the sgRNA-directed exchange of single nucleotides mediated by modified forms of Cas9 protein, Cas9 nickases (nCas9), which lack the capacity to cleave DNA, but instead are fused to bacterial deaminases to substitute single nucleotides. (see Komor A C, Kim Y B, Packer M S, Zuris J A, Liu D R. Programmable editing of a target base in genomic DNA without double-stranded DNA cleavage. Nature (2016) 533 (7603): 420-4) (see also for CAR-T cell engineering: Webber B R, Lonetree C-1, Kluesner M G, Johnson M J, Pomeroy E J, Diers M D, et al., Highly efficient multiplex human T cell engineering without double-strand breaks using Cas9 base editors. Nat Commun (2019) 10 (1): 5222.)). It is an attractive strategy to knock out genes or correct pathogenic single nucleotide polymorphisms without the need for genomic double-strand breaks or co-delivering a homology-directed repair template (HDRT). For nCas9-mediated base editing, single-strand DNA point mutations are subsequently resolved in the process of replication and are used to deliberately alter the codon sequence including insertion of premature STOP codons. Base editing requires three elements. Broadly, a Cas nickase or Cas fused to a deaminase that makes the edit, a gRNA targeting Cas to a specific locus, and a target base for editing within the editing window specified by the Cas protein.

Recently CRISPR-free all-protein base editors have been described that are also suitable (Mok B Y, Kotrys A V, Raguram A, Huang T P, Mootha V K, Liu D R. Crispr-free base editors with enhanced activity and expanded targeting scope in mitochondrial and nuclear DNA. Nat Biotechnol (2022) 40:1378-87).

Cytosine base editors (CBEs) that convert C·G to T·A base pairs (Komor A. C., Kim Y. B., Packer M. S., Zuris J. A., Liu D. R. Programmable editing of a target base in genomic DNA without double-stranded DNA cleavage. Nature. 2016; 533:420-424) and adenine base editors (ABEs) that convert A·T to G·C base pairs (Gaudelli N. M., Komor A. C., Rees H. A., Packer M. S., Badran A. H., Bryson D. I., Liu D. R. Programmable base editing of A·T to G·C in genomic DNA without DNA cleavage. Nature. 2017; 551:464-471) are thus relevant tools to knock-out genes according to the present invention (see Knipping F, Newby G A, Eide C R, et al. Disruption of HIV-1 co-receptors CCR5 and CXCR4 in primary human T cells and hematopoietic stem and progenitor cells using base editing. Mol Ther. 2022; 30 (1): 130-144)

Prime editing links nCas9 to a reverse transcriptase, both of which are guided to a desired genomic locus by a customizable prime editing guide RNA (pegRNA). It is a versatile and precise genome editing method that directly writes new genetic information into a specified DNA site using a catalytically impaired Cas9 fused to an engineered reverse transcriptase, programmed with a prime editing guide RNA (pegRNA) that both specifies the target site and encodes the desired edit. (Anzalone A V, Randolph P B, Davis J R, Sousa A A, Koblan L W, Levy J M, et al., Search-and-Replace genome editing without double-strand breaks or donor DNA. Nature (2019) 576 (7785): 149-57; Nelson J W, Randolph P B, Shen S P, Everette K A, Chen P J, Anzalone A V, et al., Engineered pegrnas improve prime editing efficiency. Nat Biotechnol (2022) 40 (3): 402-10). After binding to the target region, prime editing allows for single base exchanges as well as insertions and deletions of synthetic DNA sequences of limited length. Prime editing notably allows to incorporate DNA sequences of up to 5 kb length using a platform dubbed twin prime editing (TwinPE) (Anzalone A V, Gao X D, Podracky C J, Nelson A T, Koblan L W, Raguram A, et al., Programmable deletion, replacement, integration and inversion of Large DNA sequences with twin prime editing. Nat Biotechnol (2022) 40 (5): 731-40).

### Delivery of Nucleic Acids Encoding the Gene Disrupting Molecules and Complexes

In some embodiments, a nucleic acid encoding the DNA-targeting molecule, complex, or combination, is administered or introduced to the cell. Typically, viral and non-viral based gene transfer methods (see below) can be used to introduce nucleic acids encoding components of a CRISPR, ZFP, ZFN, TALE, and/or TALEN system to cells in culture.

In some embodiments, the polypeptides are synthesized in situ in the cell as a result of the introduction of polynucleotides encoding the polypeptides into the cell. In some aspects, the polypeptides could be produced outside the cell and then introduced thereto.

In some embodiments, the CRISPR system is introduced as a ribonucleoprotein (RNP) complex containing a Cas protein (e.g., Cas9 nuclease) and a gRNA. This format enables the swiftest genome editing as there is no need for transcription and/or translation. It also offers the most transient functionality of genome editing cassette with reduced off-target effects and toxicity.

An alternative option is to use Cas9 mRNA together with sgRNA. Cas9 mRNA can be easily obtained by in vitro transcription. Cas9 mRNA delivery provides transient expression of Cas9 protein, which may be helpful for decreasing the off-target editing events.

Another option is the plasmid-based CRISPR/Cas9 system. This is delivery approach remains simple and at low cost. Both Cas9 and sgRNA cassettes, even HDR template (i.e in some embodiments encoding for the chimeric antigen receptor and/or costimulatory polypeptide) can packed in the same plasmid, which high. However, the large genetic size of Cas9 (˜4.5 kb) and the total plasmid size (>7 kb) may raise issues of delivery and expression of CRISPR/Cas9 systems.

In some embodiments, one or more vectors driving expression of one or more elements of the CRISPR system are introduced into the cell such that expression of the elements of the CRISPR system direct formation of the CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. In some embodiments, CRISPR system elements that are combined in a single vector may be arranged in any suitable orientation. In some embodiments, the CRISPR enzyme, guide sequence, tracr-mate sequence, and tracr sequence are operably linked to and expressed from the same promoter. In some embodiments, a vector comprises a regulatory element operably linked to an enzyme-coding sequence encoding the CRISPR protein.

CRISPR RNA (crRNA, contains the RNA used by Cas9 to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). CRISPR/Cas9 often employs a plasmid to transfect the target cells. The crRNA needs to be designed for each application as this is the sequence that Cas9 uses to identify and directly bind to the target DNA in a cell. The repair template carrying CAR expression cassette need also be designed for each application, as it must overlap with the sequences on either side of the cut and code for the insertion sequence. Multiple crRNA's and the tracrRNA can be packaged together to form a single-guide RNA (sgRNA). This sgRNA can be joined together with the Cas9 gene and made into a plasmid in order to be transfected into cells.

Typically, the CRISPR system is delivered using electroporation. In some embodiment, the RNP complex is first formed and can be then electroporated into the cell. Methods, compositions, and devices for electroporation are available in the art, e.g., those described in WO2006/001614 or Kim, J. A. et al. Biosens. Bioelectron. 23, 1353-1360 (2008). Additional or alternative methods, compositions, and devices for electroporation can include those described in U.S. Patent Appl. Pub. Nos. 2006/0094095; 2005/0064596; or 2006/0087522. Additional or alternative methods, compositions, and devices for electroporation can include those described in Li, L. H. et al. Cancer Res. Treat. 1, 341-350 (2002); U.S. Pat. Nos. 6,773,669; 7,186,559; 7,771,984; 7,991,559; 6,485,961; and 7,029,916; and U.S. Patent Appl. Pub. Nos: 2014/0017213; and 2012/0088842. Additional or alternative methods, compositions, and devices for electroporation can include those described in Geng, T. et al. J. Control Release 144, 91-100 (2010); and Wang, J., et al. Lab Chip 10, 2057-2061 (2010).

Methods of non-viral delivery of nucleic acids can also be used and include lipofection, nucleofection, microinjection, biolistics, virosomes, liposomes, immunoliposomes, polycation or lipid: nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024.

In some embodiments, the CRISPR system (typically in the form of a RNP complex) is delivered using cell penetrating peptides as described for example in Heitz, F., Morris, M. C. & Divita, G. Twenty years of cell-penetrating peptides: from molecular mechanisms to therapeutics. Br. J. Pharmacol. 157, 195-206 (2009); Lee, Y.-J., Erazo-Oliveras, A. & Pellois, J.-P. Delivery of macromolecules into live cells by simple co-incubation with a peptide. ChemBioChem 11, 325-330 (2010); Ruseska, I. & Zimmer, A. Internalization mechanisms of cell-penetrating peptides. Beilstein J. Nanotechnol. 11, 101-123 (2020); Staahl B T, Benekareddy M, Coulon-Bainier C, et al. Efficient genome editing in the mouse brain by local delivery of engineered Cas9 ribonucleoprotein complexes. Nat Biotechnol. 2017; 35 (5): 431-434; Del′Guidice, T. et al. Membrane permeabilizing amphiphilic peptide delivers recombinant transcription factor and CRISPR-Cas9/Cpf1 ribonucleoproteins in hard-to-modify cells. PLOS ONE 13, e0195558 (2018); Rouet, R. et al. Receptor-mediated delivery of CRISPR-Cas9 endonuclease for cell-type-specific gene editing. J. Am. Chem. Soc. 140, 6596-6603 (2018); or recently in Foss, D. V., Muldoon, J. J., Nguyen, D. N. et al. Peptide-mediated delivery of CRISPR enzymes for the efficient editing of primary human lymphocytes. Nat. Biomed. Eng 7, 647-660 (2023).)

Delivery can be to cells (e.g. in vitro or ex vivo administration) or target tissues (e.g. in vivo administration) in particular under the form of a RNP complex.

The use of editing strategies avoiding DBS and/or delivery strategies of gene editing systems avoiding electroporation allows, notably in combination to envision multiplexing strategies of immune cells wherein multiple genes including suv39h1 and potentially TRAC, as well as any gene involved in persistence of immune cell is highly relevant. Indeed these strategies notably taken in combination allow to perfom sequential delivery of nucleases to T cells and to decrease the frequency of translocations

### Antigen-Specific Receptors

The cells of the disclosure with modulated SUV39H1 expression include immune cells that express one or more, or two or more, or three or more antigen-specific receptors on their surface, and optionally one or more co-stimulatory receptors or co-stimulatory molecule(s). Antigen-specific receptors include recombinant or modified T cell receptors (TCRs) and components thereof, and/or chimeric antigen receptors (CAR). For example, at least two CAR, at least two TCR including (TCR-like fusion molecules such as HiTCR), or at least one CAR with at least one TCR can be contemplated. The two or more antigen-specific receptors may bind the same or different antigen. In some embodiments, the two or more antigen-specific receptors have different signaling domains. In some embodiments, the cell comprises an antigen-specific receptor with an activating signaling domain and an antigen-specific receptor with an inhibitory signaling domain. Typically, such antigen-specific receptors bind the target antigen with a Kd binding affinity of about 10⁻⁶M or less, about 10⁻⁷M or less, about 10⁻⁸M or less, about 10⁻⁹M or less, about 10⁻¹⁰M or less, or about 10⁻¹¹M or less (lower numbers indicating greater binding affinity).

The cells thus may comprise one or more nucleic acids that encode one or more antigen-specific receptors, optionally operably linked to a heterologous regulatory control sequence. Typically, the nucleic acids are heterologous, (i.e., for example which are not ordinarily found in the cell being engineered and/or in the organism from which such cell is derived). In some embodiments, the nucleic acids are not naturally occurring, including chimeric combinations of nucleic acids encoding various domains from multiple different cell types. The nucleic acids and their regulatory control sequences are typically heterologous. In some embodiments, the nucleic acid encoding the antigen-specific receptor may be heterologous to the immune cell and operatively linked to an endogenous promoter of the T-cell receptor such that its expression is under control of the endogenous promoter. In some embodiments, the nucleic acid encoding a CAR is operatively linked to an endogenous TRAC promoter.

The immune cells, particularly if allogeneic, may be designed to reduce graft vs. host disease, such that the cells comprise inactivated (e.g. disrupted or deleted) endogenous TCR or have reduced functional TCR expression (at least 20%, 30%, 40%, 50%, 60%, 70%, 80%, 90%, 95%, 99% reduced expression as compared to a corresponding immune cell with non modified endogenous TCR expression). Because a single gene encodes the alpha chain (TRAC) rather than the two genes encoding the beta chain, the TRAC locus is a typical target for reducing TCR receptor expression. Thus, in some embodiments, the nucleic acid encoding the antigen-specific receptor (e.g. CAR or TCR) may be integrated into the TRAC locus at a location (for example in the 5′ region of the first exon (SEQ ID NO: 3 of WO 2017/180989, SEQ ID NO: 17 herein), that significantly reduces expression of a functional TCR alpha chain. See, e.g., Jantz et al., WO 2017/062451; Sadelain et al.; Torikai et al., Blood, 119 (2): 5697-705 (2012); Eyquem et al., Nature. 2017 Mar. 2; 543 (7643): 113-117. Expression of the endogenous TCR alpha may be reduced by at least 50%, 60%, 70%, 75%, 80%, 85%, 90%, 95%, 98%, or 99% as above mentioned. In such embodiments, expression of the nucleic acid encoding the antigen-specific receptor is optionally under control of the endogenous TCR-alpha promoter.

T cells lacking expression or having reduced expression of a functional endogenous TCR may also be produced using a variety of approaches. As a matter of example, TCR expression can be reduced or eliminated using nucleic acid molecules that block or inhibit transcription or translation, such as antisense oligonucleotides complementary to the TRAC sequence or a fragment thereof; RNA interfering agents (such as a small interfering RNA (siRNA), small hairpin RNA (shRNA), microRNA (miRNA), or a piwiRNA (piRNA); ribozymes and combinations thereof that target nucleic acids encoding specific TCRs (e.g., TCR-a and TCR-β) and/or CD3 chains in primary T cells (see for example WO 2011/059836). By blocking expression of one or more of these proteins, the T cell will no longer produce one or more of the key components of the TCR complex, thereby destabilizing the TCR complex and preventing cell surface expression of a functional TCR. Even though some TCR complexes can be recycled to the cell surface, the shRNA will prevent new production of TCR proteins resulting in degradation and removal of the entire TCR complex, resulting in the production of a T cell having a stable deficiency in functional TCR expression. Expression of shRNAs in primary T cells can be achieved using any conventional expression system, e.g., a lentiviral expression system. Although lentiviruses are useful for targeting resting primary T cells, not all T cells will express the shRNAs. Some of these T cells may not express sufficient amounts of the shRNAs to allow enough inhibition of TCR expression to alter the functional activity of the T cell. Thus, T cells that retain moderate to high TCR expression after viral transduction can be removed, e.g., by cell sorting or separation techniques, so that the remaining T cells are deficient in cell surface TCR or CD3, enabling the expansion of an isolated population of T cells deficient in expression of functional TCR or CD3.

TCR expression can also be impaired (e.g. reduced or eliminated) using gene editing technologies.

### Chimeric Antigen Receptors (CARs)

In some embodiments, the engineered antigen-specific receptors comprise chimeric antigen receptors (CARs), including activating or stimulatory CARs, costimulatory CARs (see WO2014/055668), and/or inhibitory CARs (iCARs, see Fedorov et al., Sci. Transl. Medicine, 5 (215) (December 2013)).

Chimeric antigen receptors (CARs), (also known as Chimeric immunoreceptors, Chimeric T cell receptors, Artificial T cell receptors) are engineered antigen-specific receptors, which graft an arbitrary specificity onto an immune effector cell (T cell). Typically, these receptors are used to graft the specificity of a monoclonal antibody onto a T cell, with transfer of their coding sequence facilitated by retroviral vectors.

CARs generally include an extracellular antigen (or ligand) binding domain linked to one or more intracellular signaling components, in some aspects via linkers and/or transmembrane domain(s). Such molecules typically mimic or approximate a signal through a natural antigen receptor, a signal through such a receptor in combination with a costimulatory receptor, and/or a signal through a costimulatory receptor alone.

The CAR may include (a) an extracellular antigen-binding domain, (b) a transmembrane domain, (c) optionally a co-stimulatory domain, and (d) an intracellular signaling domain.

In some embodiments, the CAR is constructed with a specificity for a particular antigen (or marker or ligand), such as an antigen expressed in a particular cell type to be targeted by adoptive cell therapy, such as a cancer marker. The CAR typically includes in its extracellular portion one or more antigen binding molecules, such as one or more antigen-binding fragment, domain, or portion of an antibody, typically one or more antibody variable domains. For example, the extracellular antigen-binding domain may comprise a light chain variable domain or fragment thereof and/or a heavy chain variable domain or fragment thereof, typically as an scFv. In some embodiments, the CAR comprises an antibody heavy chain variable domain or fragment thereof that specifically binds the antigen.

The moieties used to bind to antigen or antigen binding domain typically includes three general categories, either single-chain antibody fragments (scFvs) derived from antibodies, Fab's selected from libraries, or natural ligands that engage their cognate receptor (for the first-generation CARs). Successful examples in each of these categories are notably reported in Sadelain M, Brentjens R, Riviere I. The basic principles of chimeric antigen receptor (CAR) design. Cancer discovery. 2013; 3 (4): 388-398 (see notably table 1) and are included in the present disclosure.

Antibodies include chimeric, humanized or human antibodies, and can be further affinity matured and selected as described above. Chimeric or humanized scFvs derived from rodent immunoglobulins (e.g. mice, rat) are commonly used, as they are easily derived from well-characterized monoclonal antibodies. Humanized antibodies contain rodent-sequence derived CDR regions. Typically the rodent CDRs are engrafted into a human framework, and some of the human framework residues may be back-mutated to the original rodent framework residue to preserve affinity, and/or one or a few of the CDR residues may be mutated to increase affinity. Fully human antibodies have no murine sequence and are typically produced via phage display technologies of human antibody libraries, or immunization of transgenic mice whose native immunoglobin loci have been replaced with segments of human immunoglobulin loci. Variants of the antibodies can be produced that have one or more amino acid substitutions, insertions, or deletions in the native amino acid sequence, wherein the antibody retains or substantially retains its specific binding function. Conservative substitutions of amino acids are well known and described above. Further variants may also be produced that have improved affinity for the antigen.

In some embodiments, the modified TCR (as described below) or CAR of the present invention contains a fragment of an antibody (typically a monoclonal antibody) or an antigen-binding fragment (e.g. single domain antibody, scFv, or variable heavy (VH) region and/or variable light (VL) region or 1, 2, or 3 CDRs of such VH and/or VL) that specifically recognizes an intracellular antigen, such as a tumor-associated antigen, presented on the cell surface as a MHC-peptide complex (i.e. pMHC restricted antibodies also names TCR-like antibodies). Generally, a CAR containing an antibody or antigen-binding fragment that exhibits TCR-like specificity directed against peptide-MHC complexes also may be referred to as a TCR-like CAR.

The transmembrane domain in some embodiments is derived either from a natural or from a synthetic source. The transmembrane domain may be derived from the same receptor as the intracellular signaling domain, or a different receptor. Transmembrane regions include those derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD 134, CD137, CD154, ICOS or a GITR, or NKG2D, OX40, 2B4, DAP10, DAP12, or CD40. For T cells, CD8, CD28, CD3 epsilon may be preferred. For NK cells, NKG2D, DAP10, DAP12 may be preferred. In some embodiments, the transmembrane domain is derived from CD28, CD8 or CD3-zeta. In some instances, the transmembrane domain is selected or modified by amino acid substitution to avoid binding of such domains to the transmembrane domains of the same or different surface membrane proteins to minimize interactions with other members of the receptor complex.

In some embodiments, a short oligo- or polypeptide linker, for example, a linker of between 2 and 10 amino acids in length, is present and forms a linkage between the transmembrane domain and the cytoplasmic signaling domain of the CAR.

The CAR generally includes at least one intracellular signaling component or components. First generation CARs typically had the intracellular domain from the CD3-zeta-chain, which is the primary transmitter of signals from endogenous TCRs. Second generation CARs typically further comprise intracellular signaling domains from various costimulatory protein receptors to the cytoplasmic tail of the CAR to provide additional signals to the T cell. Co-stimulatory domains include domains derived from human CD28, 4-1BB (CD137), ICOS, CD27, OX 40 (CD134), DAP10, DAP12, 2B4, CD40, FCER1G or GITR (AITR). For T cells, CD28, CD27, 4-1BB (CD137), ICOS may be preferred. For NK cells, DAP10, DAP12, 2B4 may be preferred. Combinations of two co-stimulatory domains are contemplated, e.g. CD28 and 4-1BB, or CD28 and OX40. Third generation CARs combine multiple signaling domains, such as CD3zeta-CD28-4-1BB or CD3zeta-CD28-OX40, to augment potency.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such signaling components.

In some aspects, the CAR includes a primary cytoplasmic signaling sequence that regulates primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR zeta, FcR gamma, FcR beta, CD3 gamma, CD3 delta, CD3 epsilon, CDS, CD22, CD79a, CD79b, and CD66d. In some embodiments, cytoplasmic signaling molecule(s) in the CAR contain(s) a cytoplasmic signaling domain, portion thereof, or sequence derived from CD3 zeta. The CAR can also include a signaling domain and/or transmembrane portion of a costimulatory receptor, such as CD28, 4-1BB (CD137), ICOS, CD27, OX 40 (CD134), DAP10, DAP12, 2B4, CD40, FCER1G or GITR (AITR). In some aspects, the same CAR includes both the activating and costimulatory components; alternatively, the activating domain is provided by one CAR whereas the costimulatory component is provided by another CAR recognizing another antigen.

The intracellular signaling domain can be from an intracellular component of the TCR complex, such as a TCR CD3 chain that mediates T-cell activation and cytotoxicity, e.g., the CD3 zeta chain. Alternative intracellular signaling domains include FcεRIγ. The intracellular signaling domain may comprise a modified CD3 zeta polypeptide lacking one or two of its three immunoreceptor tyrosine-based activation motifs (ITAMs), wherein the ITAMs are ITAM1, ITAM2 and ITAM3 (numbered from the N-terminus to the C-terminus). The intracellular signaling region of CD3-zeta is residues 22-164 of SEQ ID NO: 10. ITAM1 is located around amino acid residues 61-89, ITAM2 around amino acid residues 100-128, and ITAM3 around residues 131-159. Thus, the modified CD3 zeta polypeptide may have any one of ITAM1, ITAM2, or ITAM3 inactivated, e.g. disrupted or deleted. Alternatively, the modified CD3 zeta polypeptide may have any two ITAMs inactivated, e.g. ITAM2 and ITAM3, or ITAM1 and ITAM2. Preferably, ITAM3 is inactivated, e.g. deleted. More preferably, ITAM2 and ITAM3 are inactivated, e.g. deleted, leaving ITAM1. For example, one modified CD3 zeta polypeptide retains only ITAM1 and the remaining CD3zeta domain is deleted (residues 90-164). As another example, ITAM1 is substituted with the amino acid sequence of ITAM3, and the remaining CD3zeta domain is deleted (residues 90-164). See, for example, Bridgeman et al., Clin. Exp. Immunol. 175 (2): 258-67 (2014); Zhao et al., J. Immunol. 183 (9): 5563-74 (2009); Maus et al., WO-2018/132506; Sadelain et al., WO-2019/133969, Feucht et al., Nat Med. 25 (1): 82-88 (2019).

Thus, in some aspects, the antigen binding molecule is linked to one or more cell signaling modules. In some embodiments, cell signaling modules include CD3 transmembrane domain, CD3 intracellular signaling domains, and/or other CD transmembrane domains. The CAR can also further include a portion of one or more additional molecules such as Fc receptor g, CD8, CD4, CD25, or CD16.

In some embodiments, upon ligation of the CAR, the cytoplasmic domain or intracellular signaling domain of the CAR activates at least one of the normal effector functions or responses of the corresponding non-engineered immune cell (typically a T cell). For example, the CAR can induce a function of a T cell such as cytolytic activity or T-helper activity, secretion of cytokines or other factors.

The CAR or other antigen-specific receptor can also be an inhibitory CAR (e.g. iCAR) and includes intracellular components that dampen or suppress a response, such as an immune response. Examples of such intracellular signaling components are those found on immune checkpoint molecules, including PD-1, CTLA4, LAG3, BTLA, OX2R, TIM-3, TIGIT, LAIR-1, PGE2 receptors, or EP2/4 Adenosine receptors including A2AR. In some aspects, the engineered cell includes an inhibitory CAR including a signaling domain of or derived from such an inhibitory molecule, such that it serves to dampen the response of the cell. Such CARs are used, for example, to reduce the likelihood of off-target effects when the antigen recognized by the activating receptor, e.g, CAR, is also expressed, or may also be expressed, on the surface of normal cells.

### TCRs Including TCR-Like Fusion Molecules

In some embodiments, the antigen-specific receptors include recombinant modified T cell receptors (TCRs) and/or TCRs cloned from naturally occurring T cells. Nucleic acid encoding the TCR can be obtained from a variety of sources, such as by polymerase chain reaction (PCR) amplification of naturally occurring TCR DNA sequences, followed by expression of antibody variable regions, followed by selecting for specific binding to antigen. In some embodiments, the TCR is obtained from T-cells isolated from a patient, or from cultured T-cell hybridomas. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al. (2009) Clin Cancer Res. 15:169-180 and Cohen et al. (2005) J Immunol. 175:5799-5808. In some embodiments, phage display is used to isolate TCRs against a target antigen (see, e.g., Varela-Rohena et al. (2008) Nat Med. 14:1390-1395 and Li (2005) Nat Biotechnol. 23:349-354.

A “T cell receptor” or “TCR” refers to a molecule that contains a variable alpha and beta chains (also known as TCRα and TCRβ, respectively) or a variable gamma and delta chains (also known as TCRγ and TCRδ, respectively) and that is capable of specifically binding to an antigen peptide bound to a MHC receptor. In some embodiments, the antigen-binding domain of the TCR binds its target antigen with KD affinity of about 1×10⁻⁷ or less, about 5×10⁻⁸ or less, about 1×10⁻⁸ or less, about 5×10⁻⁹ or less, about 1×10⁻⁹ or less, about 5×10⁻¹⁰ or less, about 1×10⁻¹⁰ or less, about 5×10⁻¹¹ or less, about 1×10⁻¹¹ or less, about 5×10⁻¹² or less, or about 1×10⁻¹² or less (lower numbers indicating greater binding affinity). In some embodiments, the TCR is in the αβ form. Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules. In some embodiments, a TCR also can contain a constant domain, a transmembrane domain and/or a short cytoplasmic tail (see, e.g., Janeway et ah, Immunobiology: The Immune System in Health and Disease, 3rd Ed., Current Biology Publications, p. 4:33, 1997). For example, in some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction. Unless otherwise stated, the term “TCR” should be understood to encompass functional TCR fragments thereof. The term also encompasses intact or full-length modified TCRs, including TCRs in the αβ form or γδ form. The term “TCR” also includes a TCR modified to include a VH and/or VL of an antibody (such as TCR-like fusion molecule including HiTCR).

Thus, for purposes herein, reference to a TCR includes any modified TCR or functional fragment thereof, such as an antigen-binding portion of a TCR that binds to a specific antigenic peptide bound in an MHC molecule, i.e. MHC-peptide complex. An “antigen binding portion” or “antigen-binding fragment” of a TCR, which can be used interchangeably, refers to a molecule that contains a portion of the structural domains of a TCR, but that binds the antigen (e.g. MHC-peptide complex) to which the full TCR binds. In some cases, an antigen-binding portion contains the variable domains of a TCR, such as variable alpha chain and variable beta chain of a TCR, sufficient to form a binding site for binding to a specific MHC-peptide complex, such as generally where each chain contains three complementarity determining regions.

In some embodiments, the variable domains of the TCR chains associate to form loops, or complementarity determining regions (CDRs) analogous to immunoglobulins, which confer antigen recognition and determine peptide specificity by forming the binding site of the TCR molecule and determine peptide specificity. Typically, like immunoglobulins, the CDRs are separated by framework regions (FRs) (see, e.g., Jores et al., Proc. Nat'l. Acad. Sci. U.S.A. 87:9138, 1990; Chothia et al., EMBO J. 7:3745, 1988; see also Lefranc et al., Dev. Comp. Immunol. 27:55, 2003). In some embodiments, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. In some embodiments, the variable region of the beta chain can contain a further hypervariability (HV4) region.

In some embodiments, the TCR chains contain a constant domain. For example, like immunoglobulins, the extracellular portion of TCR chains (e.g., α-chain, β-chain) can contain two immunoglobulin domains, a variable domain {e.g., Vα or Vβ; typically amino acids 1 to 116 based on Kabat numbering Kabat et al., Sequences of Proteins of Immunological Interest, US Dept. Health and Human Services, Public Health Service National Institutes of Health, 1991, 5th ed.) at the N-terminus, and one constant domain (e.g., α-chain constant domain or C_{α} or TRAC, typically amino acids 117 to 259 based on Kabat, β-chain constant domain or Cβ or TRBC, typically amino acids 117 to 295 based on Kabat) adjacent to the cell membrane. For example, in some cases, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond, making a link between the two chains. In some embodiments, a TCR may have an additional cysteine residue in each of the α and β chains such that the TCR contains two disulfide bonds in the constant domains.

In some embodiments, the TCR chains can contain a transmembrane domain. In some embodiments, the transmembrane domain is positively charged. In some cases, the TCR chains contain a cytoplasmic tail. In some cases, the structure allows the TCR to associate with other molecules like CD3. For example, a TCR containing constant domains with a transmembrane region can anchor the protein in the cell membrane and associate with invariant subunits of the CD3 signaling apparatus or complex.

Generally, CD3 is a multi-protein complex that can possess three distinct chains (gamma (γ), delta (δ), and epsilon (ε)) and the zeta-chain. For example, in mammals the complex can contain a CD3gamma chain, a CD3delta chain, two CD3epsilon chains, and a homodimer of CD3zeta chains. The CD3gamma chains are highly related cell surface proteins of the immunoglobulin superfamily containing a single immunoglobulin domain. The transmembrane regions of the CD3gamma, CD3delta, and CD3epsilon chains are negatively charged, which is a characteristic that allows these chains to associate with the positively charged T cell receptor chains and play a role in propagating the signal from the TCR into the cell. The intracellular tails of the CD3gamma, CD3delta, and CD3epsilon chains each contain a single conserved motif known as an immunoreceptor tyrosine-based activation motif or ITAM, whereas each CD3 zeta chain has three ITAMs. Generally, ITAMs are involved in the signaling capacity of the TCR complex. The CD3gamma, delta, epsilon and zeta chains together form what is known as the T cell receptor complex.

In some embodiments, modified TCR or TCR like fusion molecule include recombinant HLA-independent (or non-HLA restricted) T cell receptors (referred to as “HI-TCRs”) that bind to an antigen of interest in an HLA-independent manner. Such HI-TCRs comprise an antigen binding chain that comprises: (a) an antigen-binding domain as previously herein described that binds to an antigen in an HLA-independent manner, for example, an antigen-binding fragment of an immunoglobulin variable region; and (b) a constant domain that is capable of associating with (and consequently activating) a CD32 polypeptide. Typically, the constant domain is capable of forming a homodimer or a heterodimer with another constant domain. Typically also, the antigen binding chain is capable of associating with CD3z polypeptide. Upon binding to an antigen, the antigen binding chain is capable of activating the CD3z polypeptide associated to the antigen binding chain. Activation of the CD3z polypeptide is capable of activating an immunoresponsive cell.

Because typically TCRs bind antigen in a HLA-dependent manner, the antigen-binding domain that binds in an HLA-independent manner must be heterologous. HLA independent (modified) TCRs are notably described in International Application No. WO 2019/157454. In some embodiments, the extracellular antigen-binding domain is capable of dimerizing with another extracellular antigen-binding domain.

According to the present invention such modified TCR, designated HI-TCR or HIT-CAR herein, can comprise an extracellular antigen-binding domain which comprises a ligand for a cell-surface receptor or peptide, a receptor for a cell surface ligand, an antigen binding portion of an antibody (including antibody targeting major histocompatability complex (MHC)-presented antigens) or a fragment thereof or an antigen binding portion of a TCR. In more specific embodiments, the modified TCR has an extracellular antigen-binding domain that comprises one or two immunoglobulin variable region(s). Examples of antibodies and fragments thereof include, but are not limited to, immunoglobulins such as IgG, single domain antibodies (such as VHH or nanobodies), Fv, Fab, Fab′, Fab′-SH, F (ab′) 2; diabodies; linear antibodies; variable heavy chain (VH) regions, VHH antibodies, single-chain antibody molecules such as scFvs and single-domain antibodies; and multispecific antibodies formed from antibody fragments).

Such modified TCR can therefore comprise (a) a first antigen-binding chain comprising an antigen-binding fragment of a heavy chain variable region (VH) of an antibody; and (b) a second antigen-binding chain comprising an antigen-binding fragment of a light chain variable region (VL) of an antibody; wherein the first and second antigen-binding chains each comprise a native or variant TRAC (constant region) or fragment thereof, or a native or variant TRBC (constant region) or fragment thereof. In some embodiments, at least one of the TRAC polypeptide and the TRBC polypeptide is endogenous, typically the TRAC polypeptide, and optionally one or both of the endogenous TRAC and TRBC polypeptides is inactivated.

In some designs, the HI-TCR comprises (a) a chimeric TCR alpha chain comprising a VH or fragment thereof fused to a native or variant TRAC or fragment thereof, optionally in which amino acids of the VH (or TRAC) are removed, and (b) a chimeric TCR beta chain comprising a VL or fragment thereof fused to a native or variant TRBC or fragment thereof, optionally in which amino acids of the VL (or TRBC) are removed. In other designs, the HI-TCR comprises (a) a chimeric TCR alpha chain comprising a VL or fragment thereof fused to a native or variant TRAC or fragment thereof, optionally in which amino acids of the VL (or TRAC) are removed, and (b) a chimeric TCR beta chain comprising a VH or fragment thereof fused to a native or variant TRBC or fragment thereof, optionally in which amino acids of the VH (or TRBC) are removed. In yet other designs, the HI-TCR comprises just a VH or fragment thereof fused to a native or variant TRAC or fragment thereof, or fused to a native or variant TRBC or fragment thereof, optionally in which amino acids of the VH (or TRAC or TRBC) are removed. HI-TCR (HIT-CAR) are described in Int'l Pat. Pub. No. WO 2019/157454, incorporated by reference herein in its entirety.

In some embodiments, the modified TCR may comprise one or more heterologous polypeptides, for example, (a) VH of an antibody or a fragment or variant having at least 60%, 65%, 70%, 75%, 80%, 85%, 90%, 95%, or 98% sequence identity thereto (and preferably comprising all three CDRs, or CDRs at least 60%, 65%, 70%, 75%, 80%, 85%, 90%, 95%, or 98% identical to the parental CDRs), fused to TRBC1 (SEQ ID NO: 5) or TRBC2 (SEQ ID NO: 6), or a fragment or variant of TRBC1 (SEQ ID NO: 5), TRBC2 (SEQ ID NO: 6) or a murinized version thereof (SEQ ID NO:28-29), having at least 90% sequence identity thereto, and (b) a VL of an antibody or a fragment or variant having at least at least 60%, 65%, 70%, 75%, 80%, 85%, 90%, 95%, or 98% sequence identity thereto (and preferably comprising all three CDRs, or CDRs at least 90% identical to the parental CDRs), fused to TRAC (SEQ ID NO: 4), a fragment or variant of TRAC (SEQ ID NO: 4), or a murinized version thereof (SEQ ID NO:30-31) having at least 60%, 65%, 70%, 75%, 80%, 85%, 90%, 95%, or 98% sequence identity thereto.

The modified TCR may optionally further comprise a native or variant CD3zeta polypeptide, e.g. a modified CD3zeta polypeptide (SEQ ID NO: 7) in which one or two of the ITAM domains (e.g. ITAM2 and ITAM3) have been deleted.

In some designs, the HI-TCR comprises (a) a chimeric TCR alpha chain comprising an antigen binding domain or fragment thereof, such as a VH or fragment thereof fused to a native or variant TRAC or fragment thereof, optionally in which one to three amino acids of the VH (or TRAC) are removed, and (b) a chimeric TCR beta chain comprising an antigen binding domain or fragment thereof, such as a VL or fragment thereof fused to a native or variant TRBC or fragment thereof, optionally in which one to three amino acids of the VL (or TRBC) are removed. In other designs, the HI-TCR comprises (a) a chimeric TCR alpha chain comprising an antigen binding domain or fragment thereof such as a VL or fragment thereof fused to a native or variant TRAC or fragment thereof, optionally in which one to three amino acids of the VL (or TRAC) are removed, and (b) a chimeric TCR beta chain comprising an antigen binding domain or fragment thereof such as VH or fragment thereof fused to a native or variant TRBC or fragment thereof, optionally in which one to three amino acids of the VH (or TRBC) are removed. In yet other designs, the HI-TCR comprises just an antigen binding domain or fragment thereof, such as an scFv, a VHH, a VH or fragment thereof fused to a native or variant TRAC or fragment thereof, or fused to a native or variant TRBC or fragment thereof, optionally in which one to three amino acids of the VH (or TRAC or TRBC) are removed. HI-TCR (HIT-CAR) are described in Int'l Pat. Pub. No. WO 2019/157454, incorporated by reference herein in its entirety. Recombinant HLA-independent (or non-HLA restricted) modified TCR (referred to as “HI-TCRs”) that bind to an antigen of interest in an HLA-independent manner are described in International Application No. WO 2019/157454. Such HI-TCRs comprise an antigen binding chain that comprises: (a) a heterologous antigen-binding domain that binds to an antigen in an HLA-independent manner, for example, an antigen-binding fragment of an immunoglobulin variable region; and (b) a constant domain that is capable of associating with (and consequently activating) a CD3zeta polypeptide. Preferably, the antigen-binding domain or fragment thereof comprises: (i) a heavy chain variable region (VH) of an antibody and/or (ii) a light chain variable region (VL) of an antibody. The constant domain of the TCR is, for example, a native or modified TRAC polypeptide (SEQ ID NO: 4 or variant thereof), or a native or modified TRBC polypeptide (SEQ ID NO: 5 or 6 or variant thereof). The constant domain of the TCR is, for example, a native TCR constant domain (alpha or beta) or fragment thereof.

In certain embodiments, the extracellular antigen-binding domain comprises a heavy chain variable region (V_{H}) and/or a light chain variable region (V_{L}) of an antibody, wherein the V_{H} or the V_{L} is capable of dimerizing with another extracellular antigen binding domain comprising a VL or a VH (e.g., forming a fragment variable (Fv)). In certain embodiments, the Fv is a human Fv. In certain embodiments, the Fv is a humanized Fv. In certain embodiments, the Fv is a murine Fv. In certain embodiments, the Fv is identified by screening a Fv phage library with an antigen-Fc fusion protein.

Additional extracellular antigen-binding domains targeting an interested antigen can be obtained by sequencing an existing scFv or a Fab region of an existing antibody targeting the same antigen.

In certain embodiments, the dimerized extracellular antigen-binding domain of a presently disclosed HI-TCR is a murine Fv. In certain embodiments, the dimerized extracellular antigen-binding domain is an Fv that binds to a human tumor antigen as previously defined.

In certain embodiments, the extracellular antigen-binding domain is an Fv, and specifically binds to a human CD19 polypeptide (e.g., a human CD19 polypeptide).

In certain embodiments, the Fv comprises a heavy chain variable region (V_{H}) comprising the amino acid sequence set forth in SEQ ID NO: 36 or 40. In certain embodiments, the Fv comprises a light chain variable region (V_{L}) comprising the amino acid sequence set forth in SEQ ID NO: 37 or 40. In certain embodiments, the Fv comprises V_{H} comprising the amino acid sequence set forth in SEQ ID NO: 36 or 40 and a V_{L} comprising the amino acid sequence set forth in SEQ ID NO: 37 or 41. In certain embodiments, the extracellular antigen binding domain comprises a V_{H} comprising an amino acid sequence that is at least about 80% (e.g., at least about 85%, at least about 90%, or at least about 95%) homologous or identical to SEQ ID NO: 36 or 40. For example, the extracellular antigen-binding domain comprises a V_{H} comprising an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to SEQ ID NO: 7. In certain embodiments, the extracellular antigen-binding domain comprises a V_{H} comprising the amino sequence set forth in SEQ ID NO: 37 or 40. In certain embodiments, the extracellular antigen-binding domain comprises a V_{L} comprising an amino acid sequence that is at least about 80% (e.g, at least about 85%, at least about 90%, or at least about 95%) homologous to SEQ ID NO: 37 or 41. For example, the extracellular antigen-binding domain comprises a V_{L} comprising an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to SEQ ID NO: 37 or 41. In certain embodiments, the extracellular antigen-binding domain comprises a V_{L} comprising the amino acid sequence set forth in SEQ ID NO: 37 or 41. In certain embodiments, the extracellular antigen-binding domain comprises a V_{H} comprising an amino acid sequence that is at least about 80% (e.g., at least about 85%, at least about 90%, or at least about 95%) homologous to SEQ ID NO: 36 or 40, and a V_{L} comprising an amino acid sequence that is at least about 80% (e.g, at least about 85%, at least about 90%, or at least about 95%) homologous or identical to SEQ ID NO: 37 or 41 respectively. In certain embodiments, the extracellular antigen-binding domain comprises a V_{H} comprising the amino acid sequence set forth in SEQ ID NO: 36 or 40 and a V_{L} comprising the amino acid sequence set forth in SEQ ID NO: 37 or 41 respectively.

In certain embodiments, the extracellular antigen-binding domain is an Fv, and specifically binds to a human PSMA polypeptide (e.g., a human PSMA polypeptide).

In certain embodiments, the Fv comprises a heavy chain variable region (V_{H}) comprising the amino acid sequence set forth in SEQ ID NO: 44. In certain embodiments, the Fv comprises a light chain variable region (V_{L}) comprising the amino acid sequence set forth in SEQ ID NO: 45. In certain embodiments, the Fv comprises V_{H} comprising the amino acid sequence set forth in SEQ ID NO: 44 and a V_{L} comprising the amino acid sequence set forth in SEQ ID NO: 45. In certain embodiments, the extracellular antigen binding domain comprises a V_{H} comprising an amino acid sequence that is at least about 80% (e.g., at least about 85%, at least about 90%, or at least about 95%) homologous or identical to SEQ ID NO: 44. For example, the extracellular antigen-binding domain comprises a V_{H} comprising an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to SEQ ID NO: 44. In certain embodiments, the extracellular antigen-binding domain comprises a V_{H} comprising the amino sequence set forth in SEQ ID NO: 44. In certain embodiments, the extracellular antigen-binding domain comprises a V_{L} comprising an amino acid sequence that is at least about 80% (e.g, at least about 85%, at least about 90%, or at least about 95%) homologous to SEQ ID NO: 45. For example, the extracellular antigen-binding domain comprises a V_{L} comprising an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to SEQ ID NO: 45. In certain embodiments, the extracellular antigen-binding domain comprises a V_{L} comprising the amino acid sequence set forth in SEQ ID NO: 45. In certain embodiments, the extracellular antigen-binding domain comprises a V_{H} comprising an amino acid sequence that is at least about 80% (e.g., at least about 85%, at least about 90%, or at least about 95%) homologous to SEQ ID NO: 44, and a V_{L} comprising an amino acid sequence that is at least about 80% (e.g, at least about 85%, at least about 90%, or at least about 95%) homologous or identical to SEQ ID NO: 45 respectively. In certain embodiments, the extracellular antigen-binding domain comprises a V_{H} comprising the amino acid sequence set forth in SEQ ID NO: 44 and a V_{L} comprising the amino acid sequence set forth in SEQ ID NO: 45 respectively.

Unlike chimeric antigen receptors, which typically themselves comprise an intracellular signaling domain, the HI-TCR does not directly produce an activating signal; instead, the antigen-binding chain associates with and consequently activates a CD3zeta polypeptide (SEQ ID NO: 7).

The CD3zeta polypeptide optionally comprises an intracellular domain of a co-stimulatory molecule or a fragment thereof. Alternatively, the antigen binding domain optionally comprises a co-stimulatory domain that is capable of stimulating an immunoresponsive cell upon the binding of the antigen binding chain to the antigen. Example co-stimulatory domains include stimulatory domains, or fragments or variants thereof, from CD28 (SEQ ID NO: 8-9), 4-1BB (CD137) (SEQ ID NO: 10-11), ICOS (SEQ ID NO: 12), CD27, OX 40 (CD134) (SEQ ID NO: 13), DAP10, DAP12, 2B4, CD40, FCER1G or GITR (AITR). For T cells, CD28, CD27, 4-1BB (CD137), ICOS may be preferred. For NK cells, DAP10, DAP12, 2B4 may be preferred. Combinations of two co-stimulatory domains are contemplated, e.g. CD28 and 4-1BB, or CD28 and OX40.

The foregoing modified immune cell expressing an antigen-specific receptor, e.g. modified TCR, preferably comprises one or more further features as described herein: inactivation (e.g. mutation or inhibition) of the SUV39H1 gene, and/or inactivation of one or two ITAM domains of the CD3zeta intracellular signaling region of the antigen-specific receptor, and/or inactivation of one or both endogenous TCR chains (e.g. deletion or disruption of endogenous TCR-alpha and/or TCR-beta) and/or addition of a co-stimulatory receptor, or combinations of one, two, three or all of such features.

In some embodiments, the TCR chains contain a constant domain. For example, like immunoglobulins, the extracellular portion of TCR chains (e.g., α-chain, β-chain) can contain two immunoglobulin domains, a variable domain {e.g., Vα or Vβ; typically amino acids 1 to 116 based on Kabat numbering Kabat et al., “Sequences of Proteins of Immunological Interest, US Dept. Health and Human Services, Public Health Service National Institutes of Health, 1991, 5th ed.) at the N-terminus, and one constant domain (e.g., α-chain constant domain or Cα, typically amino acids 117 to 259 based on Kabat, or β-chain constant domain or Cβ, typically amino acids 117 to 295 based on Kabat) adjacent to the cell membrane. For example, in some cases, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond, making a link between the two chains. In some embodiments, a TCR may have an additional cysteine residue in each of the α and β chains such that the TCR contains two disulfide bonds in the constant domains.

In some embodiments, the TCR chains can contain a transmembrane domain. In some embodiments, the transmembrane domain is positively charged. In some cases, the TCR chains contain a cytoplasmic tail. In some cases, the structure allows the TCR to associate with other molecules like CD3. For example, a TCR containing constant domains with a transmembrane region can anchor the protein in the cell membrane and associate with invariant subunits of the CD3 signaling apparatus or complex.

Generally, CD3 is a multi-protein complex that can possess three distinct chains (γ, δ, and ε) in mammals and the ζ-chain. For example, in mammals the complex can contain a CD3 gamma chain, a CD3 delta chain, two CD3 epsilon chains, and a homodimer of CD3zeta chains. The CD3 gamma, CD3delta, and CD3 epsilon chains are highly related cell surface proteins of the immunoglobulin superfamily containing a single immunoglobulin domain. The transmembrane regions of the CD3 gamma, CD3 delta, and CD3 epsilon chains are negatively charged, which is a characteristic that allows these chains to associate with the positively charged T cell receptor chains. The intracellular tails of the CD3 gamma, CD3 delta, and CD3 epsilon chains each contain a single conserved motif known as an immunoreceptor tyrosine-based activation motif or ITAM, whereas each CD3zeta chain has three. Generally, ITAMs are involved in the signaling capacity of the TCR complex. These accessory molecules have negatively charged transmembrane regions and play a role in propagating the signal from the TCR into the cell. The CD3 gamma-, delta-, epsilon- and zeta-chains, together with the TCR, form what is known as the T cell receptor complex.

Yet other modified TCRs or TCR-like fusion molecules are disclosed in Int'l Pat. Pub. No. WO 2018/067993, incorporated herein by reference in its entirety, and in Baeuerle, et al. Synthetic TRUC receptors engaging the complete T cell receptor for potent anti-tumor response. Nat Commun 10, 2087 (2019). For example, any one or more, or two or more, of the alpha, beta, gamma or epsilon chains (e.g. intracellular and optionally transmembrane domains thereof) may be fused to an antibody variable region, e.g., VH and/or VL, or an scFv.

In some embodiments, the nucleic acid encoding the heterologous antigen-binding domain (e.g., VH or variant or fragment thereof, or VL or variant or fragment thereof) is inserted into the endogenous TRAC locus and/or TRBC locus of the immune cell. Optionally, the nucleic acid encoding the chimeric TCR alpha (or beta) chain is operatively linked to an endogenous promoter of the T-cell receptor such that its expression is under control of the endogenous promoter. The insertion of the nucleic acid sequence can also inactivate or disrupt the endogenous expression of a TCR comprising a native TCR alpha chain and/or a native TCR beta chain. The insertion of the nucleic acid sequence may reduce endogenous TCR expression by at least about 75%, 80%, 85%, 90% or 95%.

Other examples of antigen-specific receptors, including CARs and recombinant modified TCRs, as well as methods for engineering and introducing the receptors into cells, include those described, for example, in international patent application publication numbers WO-2000/014257, WO-2013/126726, WO-2012/129514, WO-2014/031687, WO-2013/166321, WO-2013/071154, WO-2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3 (4): 388-398; Davila et al. (2013) PLOS ONE 8 (4): e61338; Turtle et al., Curr. Opin. Immunol., 2012 October; 24 (5): 633-39; Wu et al., Cancer, 2012 Mar. 18 (2): 160-75. In some aspects, the antigen-specific receptors include a CAR as described in U.S. Pat. No. 7,446,190, and those described in International Patent Application Publication No.: WO-2014/055668 A1.

### Co-Stimulatory Receptors or Molecules

The cells of the disclosure with modified SUV39H1 expression may further comprise at least one or at least two exogenous co-stimulatory ligands. Co-stimulatory ligands include CD80, CD86, 4-1BBL, CD275, CD40L, OX40L or any combination thereof. In some embodiments, the co-stimulatory ligand is CD80 or 4-1BBL and combinations thereof.

In some embodiments, the cells comprise at least one or at least two co-stimulatory receptors. Such co-stimulatory receptors include chimeric receptors comprising a co-stimulatory ligand fused to at least one or at least two co-stimulatory molecule(s). Co-stimulatory ligands include CD80, CD86, 4-1BBL, CD275, CD40L, OX40L or any combination thereof. In some embodiments, the co-stimulatory ligand is CD80 and/or 4-1BBL. Example co-stimulatory molecules are CD28, 4-1BB, OX40, ICOS, DAP-10, CD27, CD40, NKG2D, CD2, or any combination thereof. In some embodiments, the chimeric receptor or molecule comprises a first co-stimulatory molecule that is 4-1BB and a second co-stimulatory molecule that is CD28. In some embodiments, the cell comprises a co-stimulatory receptor (i.e. a fusion polypeptide) comprising an extracellular domain and a transmembrane domain of a co-stimulatory ligand, and an intracellular domain of a first co-stimulatory molecule. In some embodiments, the co-stimulatory ligand can be selected from the group consisting of a tumor necrosis factor (TNF) family member, an immunoglobulin (Ig) superfamily member, and combinations thereof. Typically, the TNF family member can be selected from the group consisting of 4-1BBL, OX40L, CD70, GITRL, CD40L, and combination thereof. The Ig superfamily member can also be selected from the group consisting of (typically human) CD80, CD86, ICOSLG, and combinations thereof. The first co stimulatory molecule can for example be selected from the group consisting of CD28, 4-IBB, OX40, ICOS, DAP-10, CD27, CD40, NKG2D, CD2, and combinations thereof. Typically, the co stimulatory ligand is CD80 and the first co-stimulatory molecule can be selected from the group consisting of (typically human) CD28, 4-1BB, OX40, ICOS, DAP-10, CD27, CD40, NKG2D, CD2, and combinations thereof. In seome embodiments, the co-stimulatory receptor or ligand is a fusion polypepide comprising the extracellular domain of human CD80, transmembrane domain of human CD80, and an intracellular human 4-1BB domain. Examples co-stimulatory ligands, molecules and receptors (or fusion polypeptides) are described in Int'l Pat. Pub. No. WO-2021/016174, incorporated by reference herein in its entirety. Illustrative costimulatory receptors or molecules include molecules of SEQ ID NO: 32-33 and 53-54.

The cells of the disclosure with modulated SUV39H1 expression may also comprise T-cell specific engagers, such as BiTEs, or bispecific antibodies that bind not only the desired antigen but also an activating T-cell antigen such as CD3 epsilon. In some embodiments, the BiTe comprises an antigen-binding domain, e.g. scFv, linked to a T-cell recognizing domain, e.g., heavy variable domain and/or light variable domain of an anti-CD3 antibody.

### Antigens

Antigens include antigens associated with diseases or disorders, including proliferative, neoplastic, and malignant diseases and disorders, more particularly cancers. Infectious diseases and autoimmune, inflammatory or allergic diseases are also contemplated.

The cancer may be a solid cancer (solid tumor) or a “liquid tumor” such as cancers affecting the blood, bone marrow and lymphoid system, also known as tumors of the hematopoietic and lymphoid tissues, which notably include leukemia and lymphoma. Liquid tumors include for example acute myelogenous leukemia (AML), chronic myelogenous leukemia (CML), acute lymphocytic leukemia (ALL), and chronic lymphocytic leukemia (CLL), (including various lymphomas such as mantle cell lymphoma, non-Hodgkins lymphoma (NHL) Central nervous system lymphoma (CNSL), adenoma, squamous cell carcinoma, laryngeal carcinoma, gallbladder and bile duct cancers, cancers of the retina such as retinoblastoma).

Solid cancers (also called solid tumors) notably include cancer affecting an organ, optionally colon, rectum, skin, endometrium, lung (including non-small cell lung carcinoma), uterus, bones (such as Osteosarcoma, Chondrosarcomas, Ewing's sarcoma, Fibrosarcomas, Giant cell tumors, Adamantinomas, and Chordomas), liver, kidney, esophagus, stomach, bladder, pancreas, cervix, brain (such as Meningiomas, Glioblastomas, Lower-Grade Astrocytomas, Oligodendrocytomas, Pituitary Tumors, Schwannomas, and Metastatic brain cancers), ovary, breast (including triple negative breast cancer), head and neck region, testis, prostate or the thyroid gland.

Cancers include cancers affecting the blood, bone marrow and lymphoid system as described above. In some embodiments, the cancer is, or is associated, with multiple myeloma. Antigens associated with multiple myeloma include CD38, CD138, and/or CS-1. Other exemplary multiple myeloma antigens include CD56, TIM-3, CD33, CD123, and/or CD44.

Diseases also encompass infectious diseases or conditions, such as, but not limited to, viral, retroviral, bacterial, protozoal or parasitic, infections, or viral infections caused by, e.g., human immunodeficiency virus (HIV), Cytomegalovirus (CMV), Epstein-Barr virus (EBV), adenovirus, BK polyomavirus, hepatitis virus, such as hepatitis B, hepatitis C, hepatitis D, hepatitis E.

In some embodiments the extracellular antigen-binding domain binds to any of the tumor neoantigenic peptides disclosed in Int'l Pat. Pub. No. WO 2021/043804, incorporated by reference herein in its entirety. For example, the antigen-binding domain binds to any of the peptides of F or to a neoantigenic peptide comprising at least 8, 9, 10, 11 or 12 amino acids that is encoded by a part of an open reading frame (ORF) of any of the fusion transcript sequences of any one of SEQ ID NO: 118-17492 of WO 2021/043804.

Diseases also encompass autoimmune or inflammatory diseases or conditions, such as arthritis, e.g., rheumatoid arthritis (RA), Type I diabetes, systemic lupus erythematosus (SLE), inflammatory bowel disease, psoriasis, scleroderma, autoimmune thyroid disease, Grave's disease, Crohn's disease multiple sclerosis, asthma, and/or diseases or conditions associated with transplant. In such circumstances, a T-regulatory cell may be the cell in which SUV39H1 is inhibited.

In some embodiments, the antigen is a polypeptide. In some embodiments, it is a carbohydrate or other molecule. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells. In some such embodiments, a multi-targeting and/or gene disruption approach as provided herein is used to improve specificity and/or efficacy.

In some embodiments, the antigen is a universal tumor antigen. The term “universal tumor antigen” refers to an immunogenic molecule, such as a protein, that is, generally, expressed at a higher level in tumor cells than in non-tumor cells and also is expressed in tumors of different origins. In some embodiments, the universal tumor antigen is expressed in more than 30%, 40%, 50%, 60%, 70%, 75%, 80%, 85%, 90% or more of human cancers. In some embodiments, the universal tumor antigen is expressed in at least three, at least four, at least five, at least six, at least seven, at least eight or more different types of tumors. In some cases, the universal tumor antigen may be expressed in non-tumor cells, such as normal cells, but at lower levels than it is expressed in tumor cells. In some cases, the universal tumor antigen is not expressed at all in non-tumor cells, such as not expressed in normal cells. Exemplary universal tumor antigens include, for example, human telomerase reverse transcriptase (hTERT), survivin, mouse double minute 2 homolog (MDM2), cytochrome P450 1 B1 (CYP1 B), HER2/neu, p95HER2, Wilms tumor gene 1 (WT1), livin, alphafetoprotein (AFP), carcinoembryonic antigen (CEA), mucin 16 (MUC16), MUC1, prostate-specific membrane antigen (PSMA), p53 or cyclin (DI). Peptide epitopes of tumor antigens, including universal tumor antigens, are known in the art and, in some aspects, can be used to generate MHC-restricted antigen-specific receptors, such as TCRs or TCR-like CARs (see e.g. published PCT application No. WO-2011/009173 or WO-2012/135854 and published U.S. application No. US20140065708).

In some embodiments, the cancer is, or is associated, with overexpression of HER2 or p95HER2. p95HER2 is a constitutively active C-terminal fragment of HER2 that is produced by an alternative initiation of translation at methionine 611 of the transcript encoding the full-length HER2 receptor. HER2 or p95HER2 has been reported to be overexpressed in breast cancer, as well as gastric (stomach) cancer, gastroesophageal cancer, esophageal cancer, ovarian cancer, uterine endometrial cancer, cervix cancer, colon cancer, bladder cancer, lung cancer, and head and neck cancers. Patients with cancers that express the p95HER2 fragment have a greater probability of developing metastasis and a worse prognosis than those patients who mainly express the complete form of HER2. Saez et al., Clinical Cancer Research, 12:424-431 (2006).

Other antigens include orphan tyrosine kinase receptor ROR1, tEGFR, Her2, p95HER2, LI-CAM, CD19, CD20, CD22, mesothelin, CEA, Claudin 18.2, hepatitis B surface antigen, anti-folate receptor, CD23, CD24, CD30, CD33, CD38, CD44, CD70, EGFR, EGP-2, EGP-4, EPHa2, ErbB2, 3, or 4, FBP, FcRH5 fetal acetylcholine e receptor, GD2, GD3, HMW-MAA, IL-22R-alpha, IL-13R-alpha2, kdr, kappa light chain, Lewis Y, L1-cell adhesion molecule, MAGE-A1, mesothelin, MUC1, MUC16, PSCA, NKG2D Ligands, NY-ESO-1, MART-1, gp100, oncofetal antigen, ROR1, TAG72, VEGF-R2, carcinoembryonic antigen (CEA), prostate specific antigen, PSMA, Her2/neu, p95HER2, estrogen receptor, progesterone receptor, ephrinB2, CD 123, CS-1, c-Met, GD-2, and MAGE A3, CE7, Wilms Tumor 1 (WT-1), a cyclin, such as cyclin A1 (CCNA1), and/or molecules expressed by HIV, HCV, HBV or other pathogens.

In some embodiments, the cancer is, or is associated, with overexpression of HER2 or p95HER2. p95HER2 is a constitutively active C-terminal fragment of HER2 that is produced by an alternative initiation of translation at methionine 611 of the transcript encoding the full-length HER2 receptor.

HER2 or p95HER2 has been reported to be overexpressed in breast cancer, as well as gastric (stomach) cancer, gastroesophageal cancer, esophageal cancer, ovarian cancer, uterine endometrial cancer, cervix cancer, colon cancer, bladder cancer, lung cancer, and head and neck cancers. Patients with cancers that express the p95HER2 fragment have a greater probability of developing metastasis and a worse prognosis than those patients who mainly express the complete form of HER2. Saez et al., Clinical Cancer Research, 12:424-431 (2006).

Antibodies that can specifically bind p95HER2 compared to HER2 (i.e., bind p95HER2 but do not bind significantly to full length HER2 receptor) are disclosed in Sperinde et al., Clin. Cancer Res. 16, 4226-4235 (2010) and U.S. Patent Pub. No. 2013/0316380, incorporated by reference herein in their entireties. Hybridomas that produce monoclonal antibodies that can specifically bind p95HER2 compared to HER2 are disclosed in Int'l. Patent Pub. No. WO/2010/000565, and in Parra-Palau et al., Cancer Res. 70, 8537-8546 (2010). An example CAR binds the epitope PIWKFPD of p95HER2 with a binding affinity KD of 10⁻⁷ M or less, 10⁻⁸ M or less, 10⁻⁹ M or less or 10⁻¹⁰ M or less. In some embodiments, a CAR or a modified TCR (e.g. Hi-TCR) as herein described comprises VH/VL sequences as described in WO2021239965.

Rius Ruiz et al., Sci. Transl. Med. 10, eaat1445 (2018) and U.S. Patent Pub. No. 2018/0118849, incorporated by reference herein in their entireties, describe a T-cell bispecific antibody that specifically binds to the epitope PIWKFPD of p95HER2 and to the CD3 epsilon chain of the TCR. The antibody designated p95HER2-TCB consists of an asymmetric two-armed immunoglobulin G1 (IgG1) that binds monovalently to CD3 epsilon and bivalently to p95HER2. The bispecific antibody has monovalent low affinity for CD3 epsilon of about 70 to 100 nM which reduces the chances of nonspecific activation, and a higher bivalent affinity for p95HER2 of about 9 nM.

When the antigen-specific receptor specifically binds p95HER2, the disclosure provides for a modified immune cell that is further modified so that it secretes a soluble (non-membrane-bound) bispecific antibody, e.g. BiTE (bispecific soluble antibody), that binds to both HER2 and a T cell activation antigen, e.g. CD3 epsilon or the constant chain (alpha or beta) of a TCR. Expressing the bispecific antibody may treat heterogeneous tumors that express both p95HER and HER2, and/or may mitigate effects of potential tumor cell escape through p95HER2 antigen loss following treatment with CAR-T cells targeting p95HER2. See, e.g., Choi et al., “CAR-T cells secreting BiTEs circumvent antigen escape without detectable toxicity,” Nature Biotechnology, 37:1049-1058 (2019).

Beside antigens from nonmutated canonical proteins overexpressed in cancer patients, cancer antigens according to the present invention also include neoantigens, generated by cancer-specific mutations as well as noncanonical polypeptides that can be generated without the need for somatic mutations.

Noncanonical cancer or tumor polypeptides include for example peptides derived from TE (transposable elements) such as in Bonté P E, Arribas Y A, Merlotti A, et al. Single-cell RNA-seq-based proteogenomics identifies glioblastoma-specific transposable elements encoding HLA-I-presented peptides. Cell Rep. 2022; 39 (10): 110916; LTR elements as in Attig J, Young G R, Hosie L, et al. LTR retroelement expansion of the human cancer transcriptome and immunopeptidome revealed by de novo transcript assembly. Genome Res. 2019; 29 (10): 1578-1590; mid exon splicing such as in Kahles A, Lehmann K V, Toussaint N C, Hüser M, Stark S G, Sachsenberg T, Stegle O, Kohlbacher O, Sander C; Cancer Genome Atlas Research Network, et al. 2018. Comprehensive analysis of alternative splicing across tumors from 8,705 patients. Cancer Cell 34:211-224.e6; intron retention such as in Smart A C, Margolis C A, Pimentel H, He M X, Miao D, Adeegbe D, Fugmann T, Wong K K, Van Allen E M. 2018. Intron retention is a source of neoepitopes in cancer. Nat Biotechnol 36:1056-1058. 10.1038/nbt.4239, non-canonical splicing junctions such as described in Merlotti A, Sadacca B, Arribas Y A, et al. Noncanonical splicing junctions between exons and transposable elements represent a source of immunogenic recurrent neo-antigens in patients with lung cancer. Sci Immunol. 2023; 8 (80): eabm6359 or in Shah N M, Jang H J, Liang Y, et al. Pan-cancer analysis identifies tumor-specific antigens derived from transposable elements. Nat Genet. 2023; 55 (4): 631-639). In some embodiments, the tumor antigen is selected from a intracellular peptide as described in WO 2022/189626 or WO 2022/189639. In these embodiments the antigen binding domain of the antigen receptor as herein described is typically a pMHC restricted antibody (e.g. a monoclonal antibody or mAbs) or a fragment thereof. In other embodiment, the tumor antigen is a surface antigen and notably a non-canonical surface polypeptide as defined for example in WO 2022/189620. More particularly, In some embodiments the extracellular antigen-binding domain binds to any of the tumor neoantigenic peptides disclosed in Int'l Pat. Pub. No. WO 2021/043804, incorporated by reference herein in its entirety. For example, the antigen-binding domain binds to any of the peptides of SEQ ID NO: 1-117 or to a neoantigenic peptide comprising at least 8, 9, 10, 11 or 12 amino acids that is encoded by a part of an open reading frame (ORF) of any of the fusion transcript sequences of any one of SEQ ID NO: 118-17492 of WO 2021/043804 or described in any of WO 2018/234367, WO 2022/189620, WO 2022/189626, and WO-2022/189639.

In some embodiments the targeted antigen antigen has a low density on the cell surface, typically the antigen has a low density on the cell surface, typically of less than about 10,000 molecules per cell, e.g. less than about 5,000, 4,000, 3,000, 2,000, 1,000, 500, 250 or 100 molecules per cell. In some embodiments, the antigen is expressed at low density by the target cell, e.g., less than about 6,000 molecules of the target antigen per cell. In some embodiments, the antigen is expressed at a density of less than about 5,000 molecules, less than about 4,000 molecules, less than about 3,000 molecules, less than about 2,000 molecules, less than about 1,000 molecules, or less than about 500 molecules of the target antigen per cell. In some embodiments, the antigen is expressed at a density of less than about 2,000 molecules, such as e.g., less than about 1,800 molecules, less than about 1,600 molecules, less than about 1,400 molecules, less than about 1,200 molecules, less than about 1,000 molecules, less than about 800 molecules, less than about 600 molecules, less than about 400 molecules, less than about 200 molecules, or less than about 100 molecules of the target antigen per cell. In some embodiments, the antigen is expressed at a density of less than about 1,000 molecules, such as e.g., less than about 900 molecules, less than about 800 molecules, less than about 700 molecules, less than about 600 molecules, less than about 500 molecules, less than about 400 molecules, less than about 300 molecules, less than about 200 molecules, or less than about 100 molecules of the target antigen per cell. In some embodiments, the antigen is expressed at a density ranging from about 5,000 to about 100 molecules of the target antigen per cell, such as e.g., from about 5,000 to about 1,000 molecules, from about 4,000 to about 2,000 molecules, from about 3,000 to about 2,000 molecules, from about 4,000 to about 3,000 molecules, from about 3,000 to about 1,000 molecules, from about 2,000 to about 1,000 molecules, from about 1,000 to about 500 molecules, from about 500 to about 100 molecules of the target antigen per cell.

In any of the aspects or embodiments herein, the antigen-binding domain may bind the target antigen with a binding affinity Kd of 10⁻⁷ M or less, or 10⁻⁸ M or less, or 10⁻⁹ M or less (smaller numbers indicating higher affinity).

### Expression Cassettes, Vectors and Targeting Constructs

In some aspects, the genetic engineering involves introduction of a nucleic acid encoding the genetically engineered component (e.g. a transgene encodige a chimeric receptor as herein defined) or other component for introduction into the cell, such as a component encoding a gene-disruption protein or nucleic acid. Genetic modification of an immunoresponsive cell (e.g., a T cell or an NKT cell) can be accomplished by transducing a substantially homogeneous cell composition with a recombinant DNA construct.

Generally, the engineering of chimeric receptor into immune cells (e.g., T cells) requires that the cells be cultured to allow for transduction and expansion. The transduction may utilize a variety of methods, but stable gene transfer is required to enable sustained chimeric receptor expression in clonally expanding and persisting engineered cells.

In some embodiments, gene transfer is accomplished by first stimulating cell growth, e.g., T cell growth, proliferation, and/or activation, followed by transduction of the activated cells, and expansion in culture to numbers sufficient for clinical applications.

Traditional techniques have utilized a suitable expression vector, in which case the immune cells are transduced with an expression cassette or (nucleic acid) construct comprising a transgene, for example, an exogenous nucleic acid encoding a CAR or a modified TCR and in some embodiment additionally an exogenous nucleic acid encoding for a costimulatiry ligand or receptor (e.g. fusion polypeptide). Said one or more exogenous nucleic acid are typically operably linked to a promoter (e.g. enhancers). Promoters are typically located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

In certain embodiments, the nucleic acid construct further comprises a first promoter that is operably linked to the costimulatiry ligand or receptor (e.g. fusion polypeptide). In certain embodiments, the nucleic acid composition further comprises a second promoter that is operably linked to the antigen-recognizing receptor.

In certain embodiments, one or both of the first and second promoters are endogenous or exogenous.

In certain embodiments, the exogenous promoter is selected from an elongation factor (EF)-1 promoter, a CMV promoter, a SV40 promoter, a PGK promoter, and a metallothionein promoter. In certain embodiments, one or both of the first and second promoters are inducible promoters. In certain embodiment, the inducible promoter is selected from a NFAT transcriptional response element (TRE) promoter, a CD69 promoter, a CD25 promoter, and an IL-2 promoterThe vectors is typically suitable for replication and integration eukaryotes. Typical cloning vectors contain transcription and translation terminators, initiation sequences, and promoters useful for regulation of the expression of the desired nucleic acid sequence.

Known vectors include transducing viral vectors and pseudotyped viral vectors, such as retrovirus (e.g., moloney murine leukemia virus, harvey murine sarcoma virus, murine mammary tumor virus, and rous sarcoma virus), lentivirus, adenovirus, adeno-associated virus (AAV), alphavirus, vaccinia virus, poxvirus, SV40-type viruses, polyoma viruses, Epstein-Barr viruses, herpes simplex virus, papilloma virus, polio virus, foamivirus, or Semliki Forest virus vectors. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

Retroviral vectors are particularly well developed and have been used in clinical settings (Rosenberg et al., N. Engl. J. Med 323:370, 1990; Anderson et al., U.S. Pat. No. 5,399,346). A retroviral vector may be, e.g., a gammaretroviral vector or a lentivirus. A gammaretroviral vector may include, e.g., a promoter, a packaging signal (w), a primer binding site (PBS), one or more (e.g., two) long terminal repeats (LTR), and a transgene of interest, e.g., a gene encoding a CAR. A gammaretroviral vector may lack viral structural gens such as gag, pol, and env. Exemplary gammaretroviral vectors include Murine Leukemia Virus (MLV), Spleen-Focus Forming Virus (SFFV), and Myeloproliferative Sarcoma Virus (MPSV), and vectors derived therefrom. Other gammaretroviral vectors are described, e.g., in Tobias Maetzig et al., “Gammaretroviral Vectors: Biology, Technology and Application” Viruses. 2011 June; 3 (6): 677-713.

Methods of lentiviral transduction are well known. Exemplary methods are described in, e.g., Wang et al. (2012) J. Immunother. 35 (9): 689-701; Cooper et al. (2003) Blood. 101:1637-1644; Verhoeyen et al. (2009) Methods Mol Biol. 506:97-114; and Cavalieri et al. (2003) Blood. 102 (2): 497-505.

For initial genetic modification of a cell to include an chimeric antigen-recognizing receptor (e.g., a CAR, a TCR, or a TCR like fusion molecule), a retroviral vector is typically employed for transduction, however any other suitable viral vector or non-viral delivery system can be used. Typically, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S. A. 94:10319, 1997).

Suitable non-viral vectors include transposase systems, such as Sleeping Beauty transposase vectors (see, e.g., Koste et al. (2014) Gene Therapy 2014 Apr. 3.; Carlens et al. (2000) Exp Hematol 28 (10): 1137-46; Alonso-Camino et al. (2013) Mol Ther Nucl Acids 2, e93; Park et al., Trends Biotechnol. 2011 November; 29 (11): 550-557; but see also (Singh H, Manuri P R, Olivares S, et al. Redirecting specificity of T-cell populations for CD19 using the Sleeping Beauty system. Cancer Res. 2008; 68 (8): 2961-2971); Kebriaei P, Singh H, Huls M H, et al., Phase I trials using Sleeping Beauty to generate CD19-specific CAR T cells. J Clin Investig 2016; 126:3363-3376).

Non-viral systems for delivery of naked plasmids to cells also include lipofection, nucleofection, microinjection, biolistics, virosomes, lipids, cationic lipid complexes, liposomes, immunoliposomes, nanoparticle, gold particle, or polymer complex, poly-lysine conjugates, synthetic polyamino polymers, other agent-enhanced uptake of DNA, and artificial viral envelopes or virions. In some embodiments, recombinant nucleic acids are transferred into T cells via electroporation {see, e.g., Chicaybam et al, (2013) PLOS ONE 8 (3): e60298 and Van Tedeloo et al. (2000) Gene Therapy 7 (16): 1431-1437). In some embodiments, recombinant nucleic acids are transferred into T cells via transposition (see, e.g., Manuri et al. (2010) Hum Gene Ther 21 (4): 427-437; Sharma et al. (2013) Molec Ther Nucl Acids 2, e74; and Huang et al. (2009) Methods Mol Biol 506:115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346:776-777 (1990)); and strontium phosphate DNA co-precipitation (Brash et al., Mol. Cell Biol., 7:2031-2034 (1987)). In some embodiments, the antigen-recognizing receptor (i.e. chimeric receptor) including at least a CAR and/or a TCR (including modified TCR such as HiTCR) and the costimulatory molecule (e.g. the fusion polypeptide as herein described) is constructed in a single, multi cistronic expression cassette, in multiple expression cassettes of a single vector, or in multiple vectors. Examples of elements that create polycistronic expression cassette include, but is not limited to, various viral and non-viral Internal Ribosome Entry Sites (IRES, e.g., FGF-1 IRES, FGF-2 IRES, VEGF IRES, IGF-II IRES, NF-KB IRES, RUNX1 IRES, p53 IRES, hepatitis A IRES, hepatitis C IRES, pestivirus IRES, aphthovirus IRES, picornavirus IRES, poliovirus IRES and encephalomyocarditis virus IRES) and cleavable linkers (e.g, 2A peptides, e.g., P2A, T2A, E2A and F2A peptides). Combinations of retroviral vector and an appropriate packaging line are also suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464). Non-amphotropic particles are suitable too, e.g., particles pseudotyped with VSVG, RDI 14 or GALV envelope and any other known in the art.

In some embodiments, the vector incorporates an endogenous promoter such as a TCR promoter. Such a vector could provide for expression in a manner similar to that provided by an endogenous promoter, such as a TCR promoter. Such a vector can be useful, for example, if the site of integration does not provide for efficient expression of a transgene, or if disruption of the endogenous gene controlled by the endogenous promoter would be detrimental to the T cell or would result in a decrease in its effectiveness in T cell therapy. Expression of a nucleic acid sequence under the control of an endogenous or vector-associated promoter occurs under suitable conditions for the cell to express the nucleic acid, for example, growth conditions, or in the presence of an inducer with an inducible promoter, and the like. Such conditions are well understood by those skilled in the art.

Well-suited constructs including a modified TCR typically include a TRBC or TRAC sequence (which can be a native or modified TRBC or TRAC sequence, including murine sequences as described herein), a cleavable linker sequence (as defined above, but such as a 2A sequence), a TRAC and/or TRBC sequence (which can be a native or modified TRBC and/or TRAC sequence, including murine sequences as described herein). The TRBC and/or the TRAC sequence is typically fused (preferably in 5′) to a sequence coding for an antibody fragment as above described (e.g. a VH, a VH, an scFv, a single domain antibody, a VHH, etc.). Typically, the booster (co-stimulatory ligand) sequence is included in the same construct, such that in preferred embodiments, the construct further includes a cleavable linker sequence (e.g. a 2A sequence) and a booster (co-stimulatory ligand) sequence. In some embodiments, the TRAC and/or TRBC sequence in the 3′ end of the construct is fused to a cleavable linker which is also fused to the booster (co-stimulatory ligand and or costimulatory receptor CCR) sequence. The booster ((co-stimulatory ligand and/or costimulatory receptor CCR) sequence can be any one as herein described and can be notably a CD80 sequence or a CD80_4-1BBL sequence as herein described (see for example SEQ ID NO:32-33 and 52-53).

If desired, the construct can optionally be designed to include a reporter, for example, a reporter protein that provides for identification of transduced cells. Exemplary reporter proteins include, but are not limited to, fluorescent proteins, such as mCherry, green fluorescent protein (GFP), blue fluorescent protein, for example, EBFP, EBFP2, Azurite, and mKalamal, cyan fluorescent protein, for example, ECFP, Cerulean, and CyPet, and yellow fluorescent protein, for example, YFP, Citrine, Venus, and YPet. Typically, the targeting construct comprises a polyadenylation (poly A) sequence 3′ of the transgene. In a preferred embodiment, the construct comprises a polyadenylation (poly A) sequence in 3′ of the nucleic acid sequences encoding a CAR and/or a modified TCR (e.g. a Hi)-TCR).

In some embodiments, the transgene or the expression cassette is cloned into a targeting construct, which provides for targeted integration of the expression cassette or the transgene at a targeted site within the genome (e.g., at the TCR or at a the SUV39H1 locus). In particular embodiments, the targeting construct is compatible for use with a homologous recombination system suitable for targeted integration of the nucleic acid sequence (transgene) at a specific genomic site (e.g., a SUV39H1 locus or a TCR locus such as a TRAC locus, including exonic or intronic locations) within the genome of the cell. Any suitable targeting construct suitable for expression in a cell of the invention, particularly an immune cell, can be employed.

Any targeted genome editing methods can also be used to deliver the fusion polypeptide and/or the antigen-recognizing receptor disclosed herein to a cell or a subject. In certain embodiments, a CRISPR system is used to deliver the fusion polypeptide and/or the antigen-recognizing receptor disclosed herein. In certain embodiments, zinc-finger nucleases are used to deliver the fusion polypeptide and/or the antigen-recognizing receptor disclosed herein. In certain embodiments, a TALEN system is used to deliver the fusion polypeptide and/or the antigen-recognizing receptor disclosed herein.

Integration of the exogenous protein (e.g., an exogenous intracellular or cell surface protein (e.g., an exogenous Hi-tCR)) into a T cell at the gRNA target site can be typically directed by co-delivery of an HDRT which includes a left and right homology arm having homology to sequences flanking the genomic break (LHA and RHA, respectively) and surrounding the exogenous protein (e.g., an exogenous intracellular or cell surface protein (e.g., an exogenous Hi-TCR)) insert. In some embodiments, the exogenous protein (e.g., an exogenous intracellular or cell surface protein (e.g., an exogenous Hi-TCR)) is integrated in-frame at the endogenous cell surface protein locus (e.g., TRAC locus), following a self cleaving peptide (e.g., P2A, E2A, T2A, or F2A) typically when an intronic locus is targeted. This leads to expression of the CAR or exogenous protein (e.g., an exogenous intracellular or cell surface protein (e.g., an exogenous Hi-TCR)) insert while simultaneously interrupting expression of the endogenous cell surface protein (e.g., endogenous TCR).

Knock in efficiency is directly correlated to nuclear concentration of the HDRT and can be increased by delivering the HDRT with for example either recombinant viral vector or ssDNA/dsDNA hybrid Cas9 shuttle as defined for example in WO2021/183884. In some embodiments, the HDRT and/or the gRNA in a composition described herein can be introduced into the cell via viral delivery using a viral vector.

It is understood that any suitable targeting construction compatible with a homologous recombination system employed can be utilized. The AAV nucleic acid sequences that function as part of a targeting construct can be packaged in several natural or recombinant AAV capsids or particles.

Particularly useful vectors for generating a target construct that provides transgene vectorization for homologous recombination-mediated targeting include, but are not limited to, recombinant Adeno-Associated Virus (rAAV), recombinant non-integrating lentivirus (rNILV), recombinant non-integrating gamma-retrovirus (rNIgRV), single-stranded DNA (linear or circular), and the like. Such vectors can be used to introduce a transgene into an immune cell of the invention by making a targeting construct (see, for example, Miller, Hum. Gene Ther. 1 (1): 5-14 (1990); Friedman, Science 244:1275-1281 (1989); Eglitis et al., BioTechniques 6:608-614 (1988); Tolstoshev et al., Current Opin. Biotechnol. 1:55-61 (1990); Sharp, Lancet 337:1277-1278 (1991); Cornetta et al., Prog. Nucleic Acid Res. Mol. Biol. 36:311-322 (1989); Anderson, Science 226:401-409 (1984); Moen, Blood Cells 17:407-416 (1991); Miller et al., Biotechnology 7:980-990 (1989); Le Gal La Salle et al., Science 259:988-990 (1993); and Johnson, Chest 107: 77S-83S (1995); Rosenberg et al., N. Engl. J. Med. 323:370 (1990); Anderson et al., U.S. Pat. No. 5,399,346; Scholler et al., Sci. Transl. Med. 4:132-153 (2012; Parente-Pereira et al., J. Biol. Methods 1 (2): e7 (1-9) (2014); Lamers et al., Blood 117 (1): 72-82 (2011); Reviere et al., Proc. Natl. Acad. Sci. USA 92:6733-6737 (1995); Wang et al., Gene Therapy 15:1454-1459 (2008)). In a particular embodiment, the AAV particle is AAV6. In a particular embodiment, an AAV2-based targeting construct is delivered to the target cell using AAV6 viral particles. In a particular embodiment, the AAV sequences are AAV2, AAV5 or AAV6 sequences.

Well-suited AAV constructs for HIT expressing in an immunoresponsive cell according to the present application are for example described in Mansilla-Soto, J., Eyquem, J., Haubner, S. et al. HLA-independent T cell receptors for targeting tumors with low antigen density. Nat Med 28, 345-352 (2022) and have been used in the results included herein, notably for the in vivo experiments.

In some embodiments, the exogenous nucleic acid or the targeting construct comprises a 5′ homology arm and a 3′ homology arm to promote recombination of the nucleic acid sequence into the cell genome at the nuclease cleavage site.

In some embodiments, an exogenous nucleic acid can be introduced into the cell using a single-stranded DNA template. The single-stranded DNA can comprise the exogenous nucleic acid and, in preferred embodiments, can comprise 5′ and 3′ homology arms to promote insertion of the nucleic acid sequence into the nuclease cleavage site by homologous recombination. The single-stranded DNA can further comprise a 5′ AAV inverted terminal repeat (ITR) sequence 5′ upstream of the 5′ homology arm, and a 3′ AAV ITR sequence 3′ downstream of the 3′ homology arm.

The targeting construct can optionally be designed to include an element that create polycistronic expression cassette (including but not limited to, various viral and non-viral Internal Ribosome Entry Sites (IRES, e.g., FGF-1 IRES, FGF-2 IRES, VEGF IRES, IGF-II IRES, NF-kB IRES, RUNX1 IRES, p53 IRES, hepatitis A IRES, hepatitis C IRES, pestivirus IRES, aphthovirus IRES, picomavirus IRES, poliovirus IRES and encephalomyocarditis virus IRES) and cleavable linkers (e.g, 2 A peptides, e.g., P2A, T2A, E2A and F2A peptides)) directly upstream of the nucleic acid sequences encoding the transgene. In preferred embodiments, the targeting construct can optionally be designed to include a cleavable linked (e.g.: P2A, T2A, etc.) sequence directly upstream of the nucleic acid sequences encoding a therapeutic protein (e.g. an engineered antigen receptor). P2A and T2A are self-cleaving peptide sequences, which can be used for bicistronic or multicistronic expression of protein sequences (see Szymczak et al., Expert Opin. Biol. Therapy 5 (5): 627-638 (2005)).

In some embodiments, the targeting construct comprises in 5′ to 3′ order: a first viral sequence, a left homology arm, a nucleic acid sequence encoding an element that create polycistronic expression cassette (e.g. various viral and non-viral Internal Ribosome Entry Sites (IRES, e.g., FGF-1 IRES, FGF-2 IRES, VEGF IRES, IGF-II IRES, NF-kB IRES, RUNX1 IRES, p53 IRES, hepatitis A IRES, hepatitis C IRES, pestivirus IRES, aphthovirus IRES, picomavirus IRES, poliovirus IRES and encephalomyocarditis virus IRES) and cleavable linkers (e.g, 2 A peptides, e.g., P2A, T2A, E2A and F2A peptides), preferably a cleavable linker), a transgene, a polyadenylation sequence, a right homology arm and a second viral sequence. In a preferred embodiment, the targeting construct comprises in 5′ to 3′ order: a first viral sequence, a left homology arm, a nucleic acid sequence encoding a self-cleaving linker (such as the porcine teschovirus 2A), a nucleic acid sequence encoding a CAR or a modified TCR (e.g. a Hi-CTR), a polyadenylation sequence, a right homology arm and a second viral sequence. In some embodiment a nucleic acid sequence coding to a co-stimulatory ligand or receptor as herein described is also included in the targeting construct.

In some embodiments, the viral nucleic acid sequence comprises sequences of an integrative-deficient Lentivirus.

In some embodiments, the gene encoding an exogenous nucleic acid sequence of the invention can be introduced into the cell by transfection with a linearized DNA template. In some examples, a plasmid DNA encoding an exogenous nucleic acid sequence can include nuclease cleavage site (such as class II, type II, V or VI Cas nuclease) at both sides of the left homology arm such that the circular plasmid DNA is linearized and allows precise in-frame integration of exogenous DNA without backbone vector sequences (see for example Hisano Y, Sakuma T, Nakade S, et al. Precise in-frame integration of exogenous DNA mediated by CRISPR/Cas9 system in zebrafish. Sci Rep. 2015; 5:8841).

### Method of Selection

In some embodiments the modified TCR as per the present invention includes a modified TRAC and or TRBC sequence as per the endogenous TRAC or TRBc constant domain sequence. Preferably the TRAC or TRBC sequence of the modified TCR include at least a mutation or a base deletion (typically a single mutation or a single base deletion) or is a murinized sequence as previously defined. Modified TCR including said modified TRAC and/or TRBC sequence(s) can advantageously be used in selection method to select the population of cell expressing said modified TCR (through negative selection typically an antibody such as used in GMP manufacturing processes targeting the endogenous TRAC or TRBC sequence). When a TRAC or TRBC knockin strategy is further used, it is further possible not only to identify cell expressing the modified TCR but furthermore to avoid cell expressing endogenous TCR.

By selecting for modified cells that do not express the endogenous cell surface protein (e.g., endogenous TCR), the method is also enriching for cells that have the CAR or exogenous protein (e.g., an exogenous intracellular or cell surface protein (e.g., an exogenous TCR)) knockin. In some embodiments, the selection method targets and selectively pulls out the unmodified T cells that still express the endogenous cell surface protein, leaving the modified T cells that express the CAR or the exogenous protein (e.g., exogenous intracellular or cell surface protein) in the supernatant, which is also referred to as negative selection. In a negative selection, the selection method targets the undesired component (e.g., the endogenous cell surface protein that is supposed to be modified), and leaves the desired population of modified T cells untouched. In some embodiments, negative selection is more efficient (less cell loss), less cytotoxic on the cells, and faster than positive selection. In a positive selection, the selection method targets the desired component or a component that is introduced into the modified T cells (e.g., the CAR, the exogenous protein (e.g., exogenous intracellular or cell surface protein), or a protein that is co-expressed with the CAR or the exogenous protein (e.g., exogenous intracellular or cell surface protein)). Moreover, positive selection targeting the CAR or the exogenous protein can lead to T cell activation, which is detrimental for antitumor activity of the T cells. Further, positive selection targeting a protein that could be co-expressed with a CAR, e.g., a truncated EGFR, requires increasing the size of the HDRT, which can have a negative impact knockin efficiency and cell viability.

After a composition described herein that contains a Cas protein, a gRNA targeting the cell surface protein locus (e.g., TRAC locus), and an HDRT that encodes the CAR or the exogenous protein (e.g., an exogenous intracellular or cell surface protein (e.g., an exogenous TCR)) is introduced (e.g., introduced via electroporation or viral delivery) into a population of T cells and the cells are incubated for a few days for the modification to take place, the modified T cells can be selected (e.g., negatively selected) by contacting the population of T cells with antibody-coated magnetic beads, in which the antibodies on the magnetic beads target the endogenous cell surface protein (e.g., endogenous TCR). In this manner, the T cells that are not modified and still express the endogenous cell surface protein (e.g., endogenous TCR) can be separated from the modified T cells that have the endogenous cell surface protein replaced by the CAR or the exogenous protein (e.g., exogenous intracellular or cell surface protein). In cases where the endogenous cell surface protein is replaced with an exogenous protein (e.g., exogenous intracellular or cell surface protein (e.g., an exogenous recombinant TCR)), one has to ensure that the epitope recognized by the antibody is only present in the endogenous cell surface protein (e.g., endogenous TCR) and not present in the exogenous protein (e.g., an exogenous intracellular or cell surface protein (e.g., an exogenous recombinant TCR)). The antibody-coated magnetic beads bound to the unmodified T cells can then be separated from the modified T cells using a magnetic separation rack. The supernatant, which contains the modified T cells, can be collected into a separate container.

### Therapeutic Uses

The cells of the disclosure may be used in adoptive cell therapy (notably adoptive T cell therapy or adoptive NK cell therapy). In some embodiments, the use is in the treatment of cancer in a subject in need thereof, but uses also include the treatment of infectious diseases and autoimmune, inflammatory or allergic diseases. In some embodiments, the subject is suffering from a cancer or at risk of suffering from a cancer.

The cells of the disclosure are particularly beneficial when administered to treat a chronic disease, such as a chronic infectious disease, or when administered to treat refractory, relapsed or resistant cancer.

In some embodiments, the patient exhibits a cancer relapse or is likely to exhibit a cancer relapse. In some embodiments, the patient exhibits cancer metastasis or is likely to exhibit cancer metastasis. In some embodiments, the patient has not achieved sustained cancer remission after one or more prior cancer therapies. In some embodiments, the patient suffers from a cancer that is resistant or nonresponsive to one or more prior cancer therapies. In some embodiments, the patient suffers from a refractory cancer. In some embodiments, the patient is likely to exhibit a response to cell therapy that is not durable. In some embodiments, the patient is ineligible for immune checkpoint therapy or did not respond to immune checkpoint therapy. In some embodiments, the patient is ineligible for treatment with high dose of chemotherapy and/or is ineligible for treatment with high adoptive cell therapy doses.

In such methods, one or more types of modified immune cells as described herein are administered to a subject in need thereof, in an amount effective to treat the disease or disorder. For example, cells expressing one or more antigen-specific receptors as described herein (including with reduced SUV39H1 activity and optionally comprising a CAR with a single active ITAM as described herein) are administered at a dose effective to treat the disease or disorder associated with the antigen(s). Treatment of any of the diseases listed above under the “Antigen” section is contemplated.

The cells may be administered at certain doses. For example, the immune cells (e.g., T cells or NK cells) in which SUV39H1 has been inhibited may be administered to adults at doses of less than about 10⁸ cells, less than about 5×10⁷ cells, less than about 10⁷ cells, less than about 5×10⁶ cells, less than about 10⁶ cells, less than about 5×10⁵ cells or less than about 10⁵ cells. The dose for pediatric patients may be about 100-fold less. In alternative embodiments, any of the immune cells (e.g. T-cells) described herein may be administered to patients at doses ranging from about 10⁵ to about 10⁹ cells, or about 10⁵ to about 10⁸ cells, or about 10⁵ to about 10⁷ cells, or about 10⁶ to about 10⁸ cells.

The subject (i.e. patient) is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent. In some examples, the patient or subject is a validated animal model for disease, adoptive cell therapy, and/or for assessing toxic outcomes such as cytokine release syndrome (CRS). In some embodiments, said subject has a cancer, is at risk of having a cancer, or is in remission of a cancer.

In some embodiments, the cells or compositions are administered to the subject, such as a subject having or at risk for a cancer or any one of the diseases as mentioned above. In some aspects, the methods thereby treat, e.g., ameliorate one or more symptom of, the disease or condition, such as with reference to cancer, by lessening tumor burden in a cancer expressing an antigen recognized by the engineered cell.

Methods for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8 (10): 577-85). See, e.g., Themeli et al. (2013) Nat Biotechnol. 31 (10): 928-933; Tsukahara et al. (2013) Biochem Biophys Res Commun 438 (1): 84-9; Davila et al. (2013) PLOS ONE 8 (4): e61338.

Administration of at least one cell according to the disclosure to a subject in need thereof may be combined with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cell populations are administered prior to the one or more additional therapeutic agents. In some embodiments, the cell populations are administered after to the one or more additional therapeutic agents.

With reference to cancer treatment, a combined cancer treatment can include but is not limited to cancer chemotherapeutic agents, cytotoxic agents, hormones, anti-angiogens, radiolabelled compounds, immunotherapy, surgery, cryotherapy, and/or radiotherapy.

Conventional cancer chemotherapeutic agents include alkylating agents, antimetabolites, anthracyclines, topoisomerase inhibitors, microtubule inhibitors and B-raf enzyme inhibitors.

Alkylating agents include the nitrogen mustards (such as mechlorethamine, cyclophosphamide, ifosfamide, melphalan and chlorambucil), ethylenamine and methylenamine derivatives (such as altretamine, thiotepa), alkyl sulfonates (such as busulfan), nitrosoureas (such as carmustine, lomustine, estramustine), triazenes (such as dacarbazine, procarbazine, temozolomide), and platinum-containing antineoplastic agents (such as cisplatin, carboplatin, oxaliplatin).

Antimetabolites include 5-fluorouracil (5-FU), 6-mercaptopurine (6-MP), Capecitabine (Xeloda®), Cytarabine (Ara-C®), Floxuridine, Fludarabine, Gemcitabine (Gemzar®), Hydroxyurea, Methotrexate, Pemetrexed (Alimta®).

Anthracyclines include Daunorubicin, Doxorubicin (Adriamycin®), Epirubicin. Idarubicin. Other anti-tumor antibiotics include Actinomycin-D, Bleomycin, Mitomycin-C, Mitoxantrone.

Topoisomerase inhibitors include Topotecan, Irinotecan (CPT-11), Etoposide (VP-16), Teniposide or Mitoxantrone.

Microtubule inhibitors include Estramustine, Ixabepilone, the taxanes (such as Paclitaxel, Docetaxel and Cabazitaxel), and the vinca alkaloids (such as Vinblastine, Vincristine, Vinorelbine, Vindesine and Vinflunine)

B-raf enzyme inhibitors include vemurafenib (Zelboraf), dabrafenib (Tafinlar), and encorafenib (Braftovi).

Immunotherapy includes but is not limited to immune checkpoint modulators (i.e. inhibitors and/or agonists), cytokines, immunomodulating monoclonal antibodies, cancer vaccines.

Preferably, administration of cells in an adoptive T cell therapy according to the disclosure is combined with administration of immune checkpoint modulators. Examples include inhibitors of (e.g. antibodies that bind specifically to and inhibit activity of) PD-1, CTLA4, LAG 3, BTLA, OX2R, TIM-3, TIGIT, LAIR-1, PGE2 receptors, and/or EP2/4 Adenosine receptors including A2AR. Preferably, the immune checkpoint modulators comprise anti-PD-1 and/or anti-PDL-1 inhibitors (e.g., anti-PD-1 and/or anti-PDL-1 antibodies).

The present disclosure also relates to the use of a composition comprising the cells as herein described for the manufacture of a medicament for treating a cancer, an infectious disease or condition, an autoimmune disease or condition, or an inflammatory disease or condition in a subject.

### EXAMPLES

### Example 1: SUV39H1 KO CAR T Cells Better Reject Established Solid Tumors

SUV39H1 KO CAR T cells were generated via lentiviral transduction of anti-CD19 CARs (custom order lentiviral particles, Flash Therapeutics) and CrispR/Cas9 mediated inactivation of SUV39H1 in healthy donor derived T cells. Efficient inactivation of SUV39H1 was achieved in cells treated with single SUV39H1 locus-targeting gRNAs (Integrated DNA Technologies, or Synthego) compared to Mock, with no differences in CAR expression. Mock cells thus expressed anti-CD19 CAR but were not treated with single SUV39H1 locus-targeting gRNAs to inactivate SUV39H1. To investigate the effect of SUV39H1 inactivation on CAR T cell antitumor efficacy, a xenogeneic model with orthotopic lung tumors was setup. A549 lung adenocarcinoma cells were generated to ectopically express luciferase and CD19 as a molecular target. These cells were then specifically targeted by anti-CD19-CAR expressing T cells. Injection of A549-CD19-luc cells in the tail vein resulted in their delivery to the lung, where their growth was monitored by bioluminescence (IVIS, Perkin Elmer, or PhotoImager, Biospace Lab) following intraperitoneal injections of 150 mg/kg D-Luciferin (D-Luciferin potassium salt, Perkin Elmer).

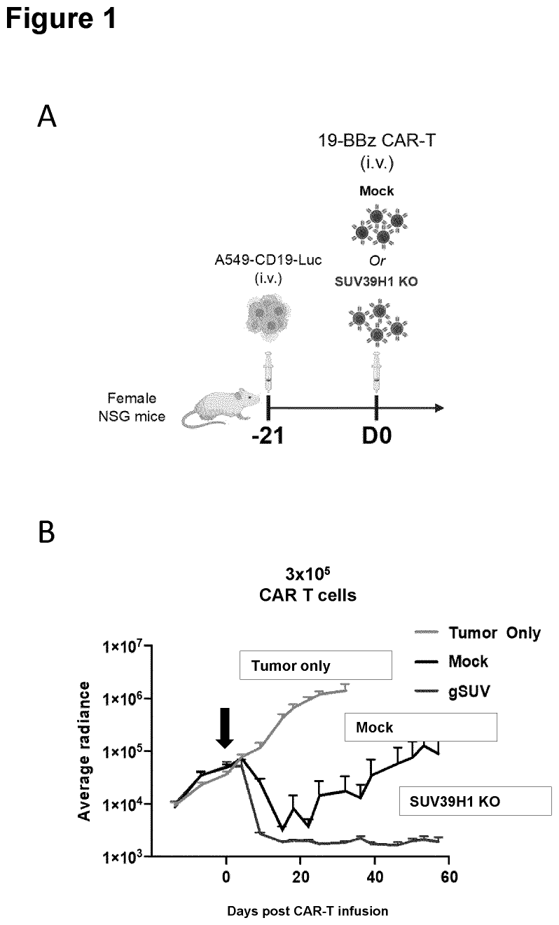

The effect of SUV39H1 inactivation on CAR T cell antitumor efficacy has been illustrated using an anti-CD19 CAR that has the following architecture: anti-CD19-scFv-CD8 (hinge and transmembrane)-4-1BB (intracellular signaling domain)-CD3zeta (intracellular signaling domain) (referenced herein as “19-BBz”), described in Maude et al, N Eng J Med, 2014. Mice were first injected with A549-CD19-luc cells and, twenty-one days later, they were infused with either 3×10⁵ Mock or 3×10⁵ SUV39H1 KO 19-BBz CAR T cells (FIG. 1A). As previously shown in Int′1. Pat. Pub. No. WO 2018/234370, SUV39H1 KO CAR T cells demonstrated faster and more long-lasting rejection of A549 orthotopic tumors (FIG. 1B).

### Example 2: Use of a CAR Construct Including CD3z Modified Intracellular Domain (1XX Construct) Further Improves Anti-Tumor Efficacy

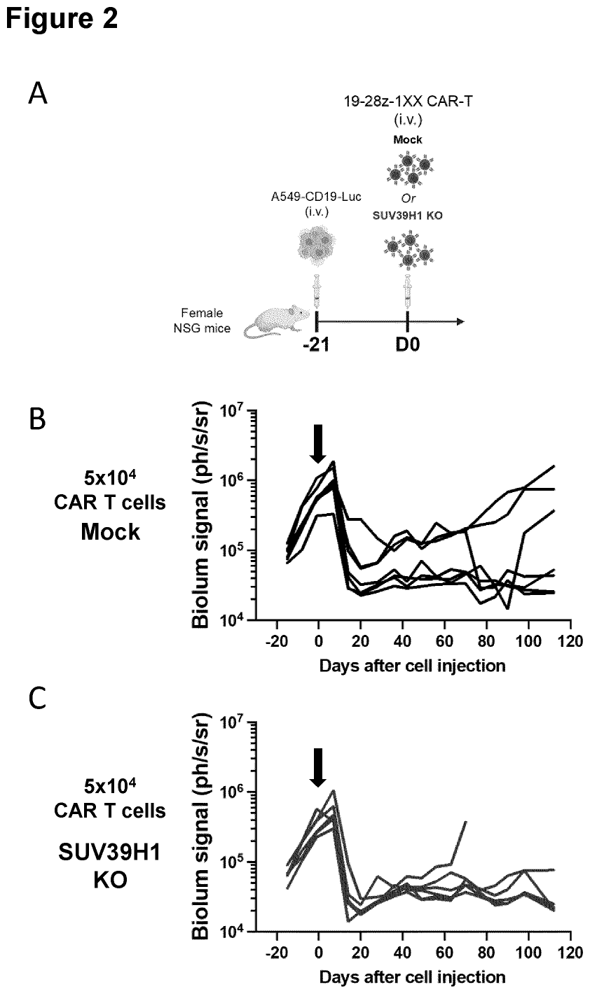

The effect of SUV39H1 inactivation on CAR T cell antitumor efficacy was tested using a different CAR structure, including a CD3z intracellular domain with inactivated ITAM2 and ITAM3). The 19-28z-1XX from Feucht et al., (Nat Med 2019) was thus expressed in SUV39H1 KO T cells. This 19-28z-1XX has the following architecture: anti-CD19-scFv-CD28 (hinge, transmembrane and intracellular signaling domain)-CD3zeta-1XX (intracellular signaling domain where ITAM2 and ITAM3 are inactivated). Mock cells expressed 19-28z-1XX CAR but were not treated to inactivate SUV39H1. Mice were first injected with A549-CD19-luc cells and, twenty-one days later, they are infused with either 5×10⁴ Mock or 5×10⁴ SUV39H1 KO 19-28z-1XX CAR T cells (FIG. 2A). As previously described in Int'l. Pat. Pub. No. WO 2021/013950, the combination of SUV39H1 KO with 1XX CAR provides superior effects. SUV39H1 KO 1 XX CAR T cells demonstrated stronger rejection of A549 orthotopic tumors (FIG. 2C) as compared to Mock 1XX CAR T cells (FIG. 2B). Moreover, 5 out of 7 mice treated with Mock CAR T cells showed persistent tumor growth, in contrast to only 2 out of 5 mice treated with SUV39H1 KO CAR T cells. Overall, it was shown that the combination of 1XX CAR construct (e.g. 19-28z-1XX) and SUV39H1 inactivation was efficacious at the lowest dose tested (5×10⁴ cells), in contrast to the combination of SUV39H1 KO and CAR constructs with the native CD3z ITAMs (e.g. 19-BBz), which did not control A549-CD19 tumors at a dose of 10⁵ cells (data not shown). Thus, replacing a CAR having three ITAMs with a 1XX CAR having a single active ITAM was able to convert a lower, ineffective dose of cells into a therapeutically effective dose of cells in a solid tumor model.

### Example 3: SUV39H1 Inactivation Increases Long-Term CAR T Cells Persistency in Peripheral Organs

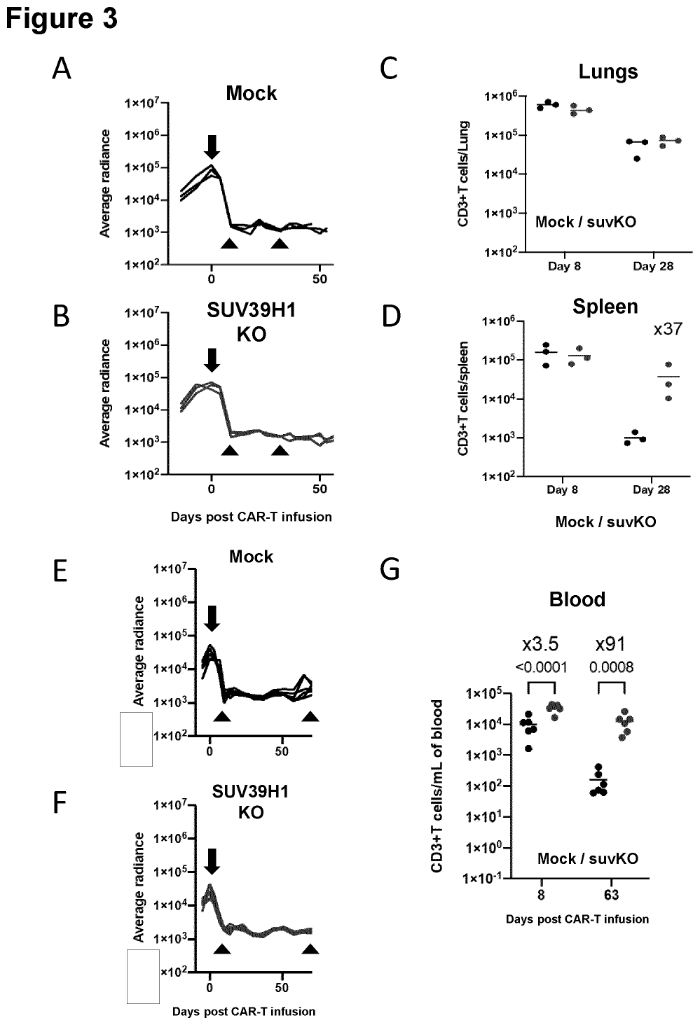

In order to study persistence of CAR T cells in vivo in peripheral organs, a higher dose (9×10⁵ cells) of either Mock or SUV39H1 KO 19-BBz CAR T cells was infused in A549-CD19 tumor bearing NSG mice. See FIGS. 3A (Mock) and 3B (SUV39H1 KO). Both Mock and SUV39H1 KO CAR T cells were able to reject established tumors at this dose (FIGS. 3A and 3B, respectively). Mice were sacrificed at different time points: a) 8 days post CAR T cell infusion, the peak of the immune response, and b) 28 days post CAR T cell infusion, when there was no residual tumor left. Immune cells were antibody-stained and then analysed by flow cytometry in a FACS analyser (BD LSRII). FIGS. 3C and 3D show CAR T cell numbers in the lungs and spleens, respectively, at day 8 and day 28. While at Day 8 there were similar numbers of either Mock or SUV39H1 KO CAR T cells in the lungs and spleens of the mice tested (approximately 1×10⁵ for Mock and SUV39H1 KO), the number of Mock cells decreased dramatically in the spleen (approximately 1×10³) by Day 28 (FIG. 3D). Therefore, SUV39H1 KO CAR T cells persist more than 10-fold better in the spleens of tumor bearing mice following tumor eradication.

The above effect could be due to better initial expansion of SUV39H1 or better overall persistence, or both. A lower dose (7.5×10⁵ cells) of either Mock or SUV39H1 KO 19-BBz CAR T cells was tested in mice, 10 days after tumor cell injection. See FIGS. 3E (Mock) and 3F (SUV39H1 KO). Both Mock and SUV39H1 KO CAR T cells were able to reject established tumors at this dose (FIGS. 3E and 3F, respectively). CAR T cell numbers were measured in peripheral blood of infused mice at different time points: a) 8 days post CAR T cell infusion, the peak of the immune response, and b) 63 days post CAR T cell infusion, when there was no residual tumor left (FIG. 3G). SUV39H1 KO CAR T cells showed stronger expansion in peripheral blood on Day 8 compared to Mock cells. On Day 63, Mock CAR T cells have dramatically decreased (approximately 1×10²) while SUV39H1 KO CAR persist close to the initial expansion levels (approximately 1×10⁴), an approximately 100-fold difference. SUV39H1 KO CAR T cells thus demonstrate both stronger initial expansion and better overall persistence.

### Example 4: SUV39H1 KO 1XX CAR T Cells Demonstrate Stronger Long-Term Persistence in Peripheral Organs

A superior effect on long term peripheral persistence has further been observed using the 1XX CAR construct (19-28z-1XX) with SUV39H1 KO, compared to Mock, over an even longer time period of 90 days after injection. A dose of 7.5×10⁵ cells of either Mock or SUV39H1 KO 19-BBz CAR T cells was infused in A549-CD19 tumor bearing NSG mice. Both Mock and SUV39H1 KO CAR T cells were able to reject established tumors at this dose. At Day 90 post CAR T cell infusion, mice were sacrificed, and the organs were analysed for CAR T cell infiltration. SUV39H1 KO 1XX CAR T cells were more abundant in both the spleens and lungs of sacrificed mice.

Therefore, SUV39H1 KO 1XX CAR T cells demonstrate stronger long-term persistence in peripheral organs following tumor eradication.

### Example 5: SUV39H1 KO CAR T Cells Better Protect Against Multiple Tumor Rechallenges

A significant drawback of current CAR T cell therapies is the inability to control tumor relapses. In the A549-CD19 xenogeneic model, this was tested by re-injecting tumor cells in the mice following primary tumor rejection by CAR T cells and then following tumor growth by bioluminescence.

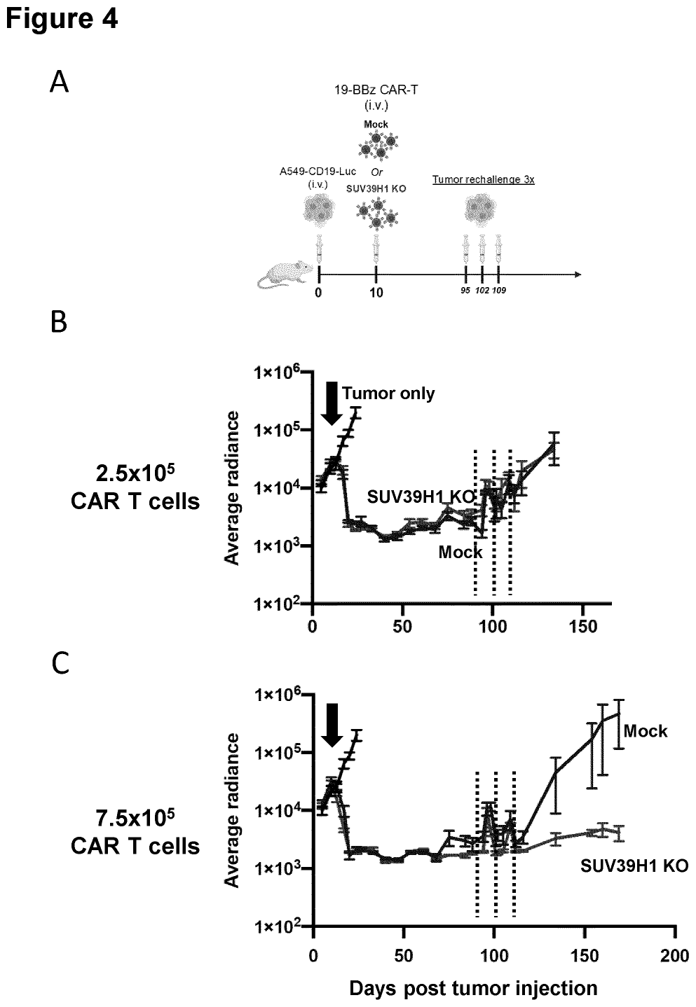

As shown in FIG. 4A, mice were first injected with A549-CD19-luc cells (DO) and ten days later, they were infused with either Mock or SUV39H1 KO 19-BBz CAR T cells. Two doses were tested: 2.5×10⁵ (FIG. 4B) and 7.5×10⁵ CAR T cells (FIG. 4C). At these doses, both Mock and SUV39H1 KO CAR T cells were able to reject primary tumors (FIGS. 4B, 4C). Tumor rechallenges were performed three times for both CAR T doses for three consecutive weeks, on Days 95, 102, and 109 post initial tumor infusion (85, 92, and 99 post CAR T infusion). At the lower CAR T cell dose of 2.5×10⁵ cells, neither Mock nor SUV39H1 CAR T cells were able to control tumor relapses following rechallenge of the mice (FIG. 4B). However, at the dose of 7.5×10⁵ CAR T cells, SUV39H1 KO cells were able to completely control rechallenges, in contrast to Mock cells which did not (FIG. 4C). These results provide strong evidence that, SUV39H1 KO CAR T cells (e.g., 19-BBz CAR T cells) are superior at protecting against tumor relapses.

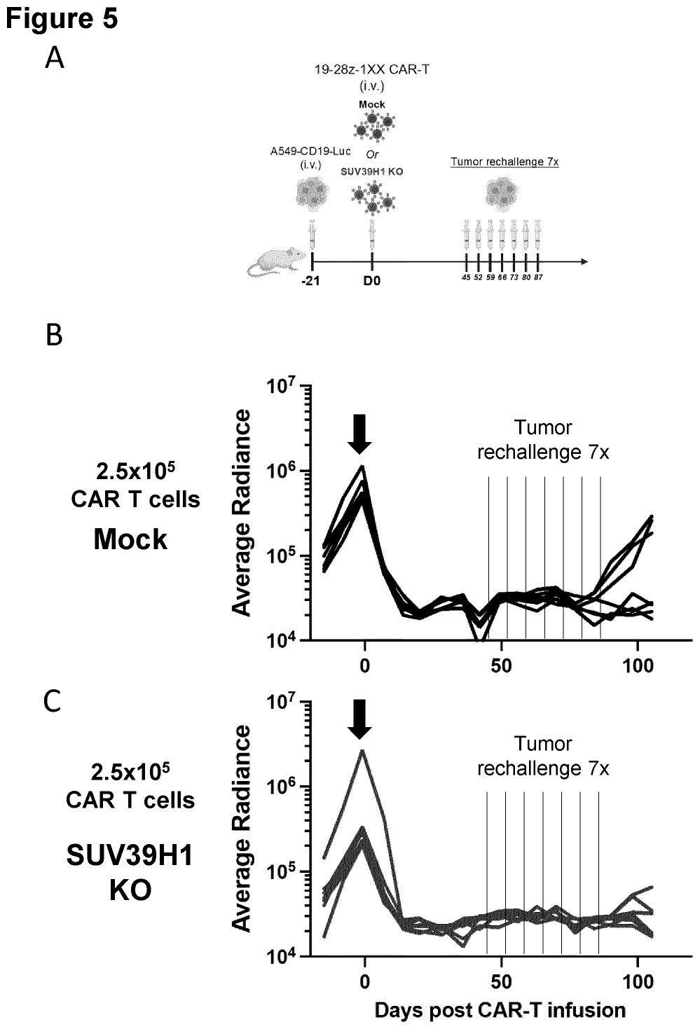

The combination of 19-28z-1XX and SUV39H1 inactivation was also tested in a rechallenge model (schematic depicted in FIG. 5A). Briefly, mice were first injected with A549-CD19-luc cells (D-21), and twenty-one days later, they were infused with either Mock 19-28z-1XX CAR T cells (FIG. 5B) or SUV39H1 KO 19-28z-1XX CAR T cells (FIG. 5C), in both cases at a dose of 2.5×10⁵ cells (DO). Both Mock and SUV39H1 KO CAR T cells were able to reject primary tumors (FIGS. 5B, 5C). Tumor rechallenges were performed seven times for seven consecutive weeks, on Days 45, 52, 59, 66, 73, 80, and 87 post CAR T infusion. SUV39H1 KO 19-28z-1XX CAR T cells demonstrate complete control of tumor rechallenges (no mice have relapses), in contrast to Mock cells, where 3 out 7 mice show tumor relapses following rechallenge (FIGS. 5B, 5C). Therefore, SUV39H1 KO+1XX CAR T cells are superior at protecting against tumor relapses. Notably, this dose of 2.5×10⁵ cells was ineffective when tested for SUV39H1 KO CAR T cells expressing conventional CARs comprising three ITAMs.

Overall, these results show that the combination of 1XX CAR construct (e.g. 19-28z-1XX) and SUV39H1 inactivation is efficacious at a lower dose (e.g. 2.5×10⁵ cells) in contrast to SUV39H1 KO cells expressing CAR constructs with the native CD3z ITAMs (e.g. 19-BBz) cells, and against even more tumor rechallenges. Thus, replacing a CAR having three ITAMs with a 1XX CAR having a single active ITAM was able to convert a lower, ineffective dose of cells into a therapeutically effective dose of cells in a 7× tumor rechallenge model that mimics a relapsing solid tumor.

All patents, patent applications, and publications are herein incorporated by reference in their entirety for all purposes and to the same extent as if each individual publication was specifically and individually indicated to be incorporated by reference in its entirety for any and all purposes.

While the foregoing articles and methods of this disclosure have been described in terms of preferred embodiments, and optional features, it will be apparent to those skilled in the art that variations or combinations may be applied without departing from the spirit and scope of the disclosure. Such variations and combinations are intended to be within the meaning and range of the disclosure as defined by the claims. The breadth and scope of the present disclosure should not be limited by any of the above-described exemplary aspects but should be defined only in accordance with the following claims and their equivalents. The terms and expressions which have been employed are used as terms of description and not of limitation, and there is no intention that in the use of such terms and expressions of excluding any equivalents of the features shown and described or portions thereof.

### Example 6: Treatment with SUV39H1-KO CAR T Cells Extends Mouse Survival in Peripheral Central Nervous System Lymphoma

Peripheral central nervous system lymphoma (PCNSL) is a B-cell derived brain tumor localized in various parts of the brain that currently has poor prognosis. 19-BBz CAR T cells have been used for the treatment of PCNSL but, despite the initial positive responses, PCNSL patients mostly relapse (Alcantara et al, Blood. 2022 Feb. 3; 139 (5): 792-796. doi: 10.1182/blood.2021012932).

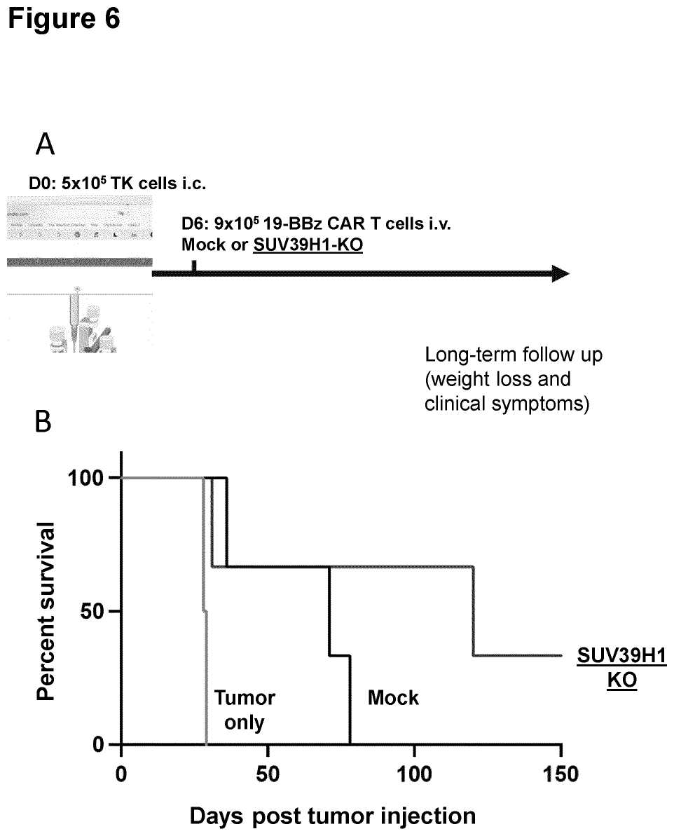

A xenogeneic model was setup in NSG mice using the human PCNSL cell line TK, which was injected intracranially (FIG. 6A). Since these cells do not express luciferase, tumor progession was followed indirectly via weight loss and clinical symptoms. As shown in FIG. 6A, the tumor-bearing mice were injected with either Mock or SUV39H1 KO 19-BBz CAR T cells six days later (dose: 9×10⁵ cells injected intravenously). Treament with SUV39H1 KO 19-BBz CAR T cells extended mouse survival compared with treatment mock 19-BBz CAR T cells. These results provide evidence that SUV39H1 inactivation prevents relapse and enhances treatment of brain tumors.

1. A modified immune cell with reduced SUV39H1 activity, for use in the treatment of a patient suffering from a refractory, relapsed or resistant cancer or suffering from a chronic infectious disease, wherein the cell expresses one or more engineered antigen-specific receptors that bind an antigen associated with the cancer or the chronic infectious disease.

2. A modified immune cell for use according to claim 1 wherein the patient suffers from a cancer, wherein said patient exhibits a cancer relapse or is likely to exhibit a cancer relapse, exhibits cancer metastasis or is likely to exhibit cancer metastasis, has not achieved sustained cancer remission after one or more prior cancer therapies, suffers from a cancer that is resistant or nonresponsive to one or more prior cancer therapies, suffers from a refractory cancer, is likely to exhibit a response to cell therapy that is not durable, is ineligible for immune checkpoint therapy or did not respond to immune checkpoint therapy, is ineligible for treatment with high dose of chemotherapy and/or is ineligible for treatment with high adoptive cell therapy doses.

3. A modified immune cell for use according to any of claim 1 or 2, wherein the antigen is orphan tyrosine kinase receptor ROR1, tEGFR, Her2, p95HER2, LI-CAM, CD19, CD20, CD22, mesothelin, CEA, Claudin 18.2, hepatitis B surface antigen, anti-folate receptor, CD23, CD24, CD30, CD33, CD38, CD44, EGFR, EGP-2, EGP-4, CD70, EPHa2, ErbB2, 3, or 4, FcRH5, FBP, fetal acethycholine e receptor, GD2, GD3, HMW-MAA, IL-22R-alpha, IL-13R-alpha2, kdr, kappa light chain, BCMA, Lewis Y, MAGE-A1, mesothelin, MUC1, MUC16, PSCA, NKG2D Ligands, NY-ESO-1, MART-1, gplOO, oncofetal antigen, TAG72, VEGF-R2, carcinoembryonic antigen (CEA), prostate specific antigen (PSMA), estrogen receptor, progesterone receptor, ephrinB2, CD 123, CS-1, c-Met, GD-2, MAGE A3, CE7, or Wilms Tumor 1 (WT-1); or optionally any of the tumor neoantigenic peptides disclosed in Int'l Pat. Pub. No. WO 2021/043804.

4. A modified immune cell for use according to any of claims 1 to 3 wherein the cancer is a myeloid or a lymphoid cancer.

5. A modified immune cell for use according to any of claims 1 to 3 wherein the cancer is a solid tumor.

6. A modified immune cell for use according to claim 5 wherein the solid tumor is a cancer affecting an organ, optionally colon, rectum, skin, endometrium, lung (including non-small cell lung carcinoma), uterus, bones (such as Osteosarcoma, Chondrosarcomas, Ewing's sarcoma, Fibrosarcomas, Giant cell tumors, Adamantinomas, and Chordomas), liver, kidney, esophagus, stomach, bladder, pancreas, cervix, brain (such as Meningiomas, Glioblastomas, Lower-Grade Astrocytomas, Oligodendrocytomas, Pituitary Tumors, Schwannomas, and Metastatic brain cancers), ovary, breast (such as triple negative, or luminal breast cancer), head and neck region, testis, prostate or the thyroid gland.

7. A modified immune cell for use according to claim 1 wherein the patient suffers from a chronic viral infection, and optionally wherein the immune cell allows long term infection control and/or increased viral clearance.

8. A modified immune cell for use according to claim 7 wherein the chronic viral infection is caused by human immunodeficiency virus (HIV), Cytomegalovirus (CMV), Epstein-Barr virus (EBV), adenovirus, BK polyomavirus, hepatitis virus, such as hepatitis B, hepatitis C, hepatitis D, hepatitis E.

9. A modified immune cell for use according to any one of claims 1 to 8, wherein the SUV39H1 activity is inhibited.

10. A modified immune cell for use according to claim 9 which has been contacted with an exogenous SUV39H1 inhibitor, optionally a nucleic acid inhibitor of SUV39H1.

11. A modified immune cell for use according to claim 10, wherein the exogenous SUV39H1 inhibitor is (a) a dominant negative inhibitor, or (b) an RNAi, shRNA, ribozyme or antisense oligonucleotide complementary to a fragment of the SUV39H1 gene, or (c) an epipolythiodioxopiperazine (ETP) class of SUV39H1 inhibitor.

12. A modified immune cell for use according to any one of claims 1 to 8, wherein the cell's SUV39H1 gene comprises one or more mutations that results in a deleted or non-functional SUV39H1 protein, or a SUV39H1 protein with reduced activity.

13. A modified immune cell for use according to any one of claims 1 to 12, wherein the antigen-specific receptor is a modified TCR.

14. A modified immune cell for use according to any one of claims 1 to 12, wherein the antigen-specific receptor is a chimeric antigen receptor (CAR).

15. A modified immune cell for use according to any one of claims 1 to 14, wherein the cell expresses at least one antigen-specific receptor having an intracellular signaling domain wherein one or two immunoreceptor tyrosine-based activation motifs (ITAMs) are inactivated, optionally wherein the antigen-specific receptor comprises a single active ITAM domain.

16. A modified immune cell for use according to any one of claims 1 to 14, wherein the antigen-specific receptor is a chimeric antigen receptor (CAR) comprising:
a) an extracellular antigen-binding domain that specifically binds an antigen, b) a transmembrane domain,
c) optionally one or more costimulatory domains, and
d) an intracellular signaling domain wherein one or two immunoreceptor tyrosine-based activation motifs (ITAMs) are inactivated, optionally wherein the antigen-specific receptor comprises a single active ITAM domain, optionally an intracellular domain comprising a modified CD3zeta intracellular signaling domain in which ITAM2 and ITAM3 have been inactivated.

17. A modified immune cell for use according to any one of claims 1 to 16, wherein the antigen-specific receptor comprises an extracellular antigen-binding domain which is an scFv; or an antibody heavy chain region (VH) and/or an antibody variable region (VL); or optionally a bispecific or trispecific antigen-binding domain.

18. A modified immune cell for use according to any one of claims 1 to 17, wherein the transmembrane domain is from CD28, CD8 or CD3-zeta, or a fragment thereof.

19. A modified immune cell for use according to any one of claims 1 to 18, wherein the one or more costimulatory domains are selected from the group consisting of: 4-1BB (CD137), CD28, CD27, ICOS, OX40 (CD134) and DAP10; DAP12, 2B4, CD40, FCER1G and/or GITR (AITR), or an active fragment thereof.

20. A modified immune cell for use according to any one of claims 1 to 16, wherein the antigen-specific receptor comprises:
a) an extracellular antigen-binding domain that specifically binds an antigen, optionally comprising an antibody heavy chain variable region and/or an antibody light chain variable region, and is optionally bispecific or trispecific;
b) a transmembrane domain, optionally comprising a fragment of transmembrane domain of alpha, beta or zeta chain of the T-cell receptor, CD28, CD3 epsilon, CD45, CD4, CD5, CD8, CD9, CD16, CD22, CD33, CD37, CD64, CD80, CD86, CD34, CD137, or CD154, NKG2D, OX40, ICOS, 2B4, DAP10, DAP12, CD40; and
c) optionally one or more co-stimulatory domains from 4-1BB, CD28, ICOS, OX40, DAP10 or DAP12, 2B4, CD40, FCER1G, or an active fragment thereof;
d) an intracellular signaling domain comprising an intracellular signaling domain from CD3zeta, FcR gamma, FcR beta, CD3 gamma, CD3 delta, CD3 epsilon, CDS, CD22, CD79a, CD79b, or CD66d, 2B4, or an active fragment thereof.

21. A modified immune cell for use according to any one of claims 1 to 16 wherein the antigen-specific receptor is a chimeric antigen receptor (CAR) comprising:
a) an extracellular antigen-binding domain, optionally an scFv,
b) a transmembrane domain, optionally from CD28, CD8 or CD3-zeta,
c) one or more co-stimulatory domains, optionally from 4-1 BB, CD28, ICOS, OX40 or DAP10, and
d) an intracellular signaling domain from CD3zeta, optionally in which ITAM2 and ITAM3 have been inactivated.

22. A modified immune cell for use according to any one of claims 1 to 16, wherein the antigen-specific receptor is a modified TCR that comprises a heterologous extracellular antigen-binding domain that specifically binds an antigen, optionally comprising an antibody heavy chain variable region and/or an antibody light chain variable region, and is optionally bispecific or trispecific.

23. A modified immune cell for use according to any one of claims 1 to 16, wherein the antigen-specific receptor is a modified TCR that comprises a fragment of an alpha, beta, gamma or epsilon chain and a heterologous extracellular antigen-binding domain that specifically binds an antigen, optionally comprising an antibody heavy chain variable region and/or an antibody light chain variable region, optionally an scFv, and is optionally bispecific or trispecific.

24. A modified immune cell for use according to any one of claims 1 to 16, wherein the antigen-specific receptor is a modified TCR that comprises:
a) a first antigen-binding chain comprising an antigen-binding fragment of a heavy chain variable region (VH) of an antibody; and
b) a second antigen-binding chain comprising an antigen-binding fragment of a light chain variable region (VL) of an antibody;
wherein the first and second antigen-binding chains each comprise a TRAC polypeptide or a TRBC polypeptide, optionally wherein at least one of the TRAC polypeptide and the TRBC polypeptide is endogenous, and optionally wherein one or both of the endogenous TRAC and TRBC polypeptides is inactivated.

25. A modified immune cell for use according to any one of claims 1 to 23 wherein a heterologous nucleic acid sequence encoding the antigen-specific receptor, or a portion thereof is inserted into the cell genome to express the antigen-specific receptor.

26. A modified immune cell for use according to any one of claims 1 to 23 wherein a heterologous nucleic acid sequence outside the cell genome expresses the antigen-specific receptor.

27. A modified immune cell for use according to claim 25, wherein insertion of the heterologous nucleic acid sequence encoding the antigen-specific receptor, or a portion thereof inactivates expression of a native TCR alpha chain and/or a native TCR beta chain.

28. A modified immune cell for use according to any of claims 1 to 27, wherein expression of the antigen-specific receptor is under control of an endogenous promoter of a TCR, optionally an endogenous TRAC promoter.

29. A modified immune cell for use according to any of claims 1 to 28, wherein the cell is a T cell, a CD4+ T cell, a CD8+ T cell, a CD4+ and CD8+ T cell, a NK cell, a T regulatory cell, a TN cell, a memory stem T cell (T_{SCM}), a T_{CM} cell, a T_{EM} cell, a monocyte, a dendritic cell, or a macrophage, or a progenitor thereof, optionally a T cell progenitor, a lymphoid progenitor, an NK cell progenitor, a myeloid progenitor, a pluripotent stem cell, an induced pluripotent stem cell (iPSC), a hematopoietic stem cell (HSC), an adipose derived stem cell (ADSC), or a pluripotent stem cell of myeloid or lymphoid lineage.

30. A modified immune cell for use according to any of claims 1 to 29, wherein the immune cell is a T cell or NK cell, or progenitor thereof.

31. A modified immune cell for use according to any of claims 1 to 30, that further comprises a second engineered antigen-specific receptor, optionally a modified TCR or CAR, that specifically binds to a second antigen.

32. A modified immune cell for use according to any of claims 1 to 30, that comprises a first CAR that binds the antigen and a second CAR that binds a second antigen.

33. A modified immune cell for use according to any of claims 1 to 30, that comprises a CAR that binds the antigen and modified TCR that binds a second antigen.

34. A modified immune cell for use according to any of claims 1 to 30, that comprises a first modified TCR that binds the antigen and a second modified TCR that binds a second antigen.

35. A modified immune cell for use according to any of claims 1 to 30, that comprises three or more engineered antigen-specific receptors.

36. A modified immune cell for use according to any of claims 1 to 35 that further comprises a heterologous co-stimulatory receptor.

37. A modified immune cell for use according to claim 36, wherein the co-stimulatory receptor comprises (a) an extracellular domain of a co-stimulatory ligand, optionally from CD80, (b) a transmembrane domain, optionally from CD80, and (c) an intracellular domain of a co-stimulatory molecule, optionally CD28, 4-1BB, OX40, ICOS, DAP10, CD27, CD40, NKGD2, or CD2, preferably 4-1BB.

38. A modified immune cell for use according to any of claims 1 to 37, wherein the extracellular antigen-binding domain binds an antigen with a KD affinity of about 1×10⁻⁷ or less, about 5×10⁻⁸ or less, about 1×10⁻⁸ or less, about 5×10⁻⁹ or less, about 1×10⁻⁹ or less, about 5×10⁻¹⁰ or less, about 1×10⁻¹⁰ or less, about 5×10⁻¹¹ or less, about 1×10⁻¹¹ or less, about 5×10⁻¹² or less, or about 1×10⁻¹² or less.

39. A modified immune cell for use according to any of claims 1 to 38, wherein the antigen has a low density on the cell surface, of less than about 10,000, or less than about 5,000, or less than about 2,000 molecules per cell.

40. A modified immune cell for use according to any of claims 1 to 39, wherein SUV39H1 expression is reduced or inhibited by at least about 50%, 60%, 70%, 75%, 80%, 85%, 90% or 95%

41. A modified immune cell for use according to any of claims 1 to 40, wherein endogenous TCR expression is reduced by at least about 75%, 80%, 85%, 90% or 95%.

42. A modified immune cell for use according to any of claims 1 to 41 that is allogeneic.

43. A modified immune cell for use according to any of claims 1 to 41 that is autologous.

44. A modified immune cell for use according to any of claims 1 to 42, wherein the HLA-A locus is inactivated.

45. A modified immune cell for use according to claim 44, wherein HLA class I expression is reduced by at least about 75%, 80%, 85%, 90% or 95%.

46. A method of producing the cell of any of claims 1 to 45 comprising (a) introducing into the cell (a) a SUV39H1 inhibitor, and (b) (i) a nucleic acid encoding a CAR having one active ITAM or (ii) a heterologous nucleic acid encoding a modified TCR.

47. The method of claim 46 wherein the heterologous nucleic acid encoding a modified TCR or a portion thereof in cloned in a vector,
optionally wherein the vector further comprise a nucleic acid encoding for a costimulatory ligand or receptor optionally a fusion polypeptide, optionally wherein the heterologous nucleic acid encoding a modified TCR or a portion and the nucleic acid encoding for a costimulatory ligand or receptor are operably linked to the same or different promoter, optionally wherein the vector is a retroviral vector, optionally a gamma retro viral vector or a lentiviral vector
optionally wherein the SUV inhibitor is a gene editing system notably a CRISPR Cas system, a base editing system or a prime editing system, optionally wherein the gene editing system is in the form of a RNP complex, optionally wherein the RNP gene editing complex is introduced in the cell using non viral delivery and without using electroporation, optionally wherein cell penetrating peptide is used
optionally wherein the cell is further modified to impair expression of a functional endogenous TCR, optionally through a TRAC KO,
optionally wherein steps a and b are performed sequentially in any order or simultaneously, optionally wherein step b is performed first.

48. A modified immune cell for use according to any of claims 1 to 45, wherein the immune cell is a CAR T-cell and a dose of less than about 5×10⁷ cells, optionally about 10⁵ to about 10⁷ cells, is administered to the subject.

49. A modified immune cell for use according to any of claims 1 to 45, wherein a second therapeutic agent, optionally one or more cancer chemotherapeutic agents, cytotoxic agents, cancer vaccines, hormones, anti-angiogens, radiolabelled compounds, immunotherapy, surgery, cryotherapy, and/or radiotherapy, is administered to the subject

50. A modified immune cell for use according to any of claims 1 to 45, wherein the second therapeutic agent is an immune checkpoint modulator.

51. A modified immune cell for use according to claim 50, wherein the immune checkpoint modulator is an antibody that specifically binds to, or other inhibitor of, PD1, PDL1, CTLA4, LAG3, BTLA, OX2R, TIM-3, TIGIT, LAIR-1, PGE2 receptor, EP2/4 adenosine receptor, or A2AR, optionally an anti-PD1 or anti-PDL1 antibody.
